SEQUENCE LISTING (146 sequences)

SEQ ID NO: 1 - DNA, 3630 bp, Homo sapiens
ggtattccagaagtgtttgaggatcccttccatgaaggaagagaggaaagtttttaagtaaacctcccactcccatgtgtcttcagctttcttttgcaaaggagaaaatccttgaagtttggtaaagaccgagttagtctatctctctttgcctatctcgagttgggctggggagaggaggagataggttcttttgtctttttctgtcttctcccttccccacttccttccctccagtccccactcactcacatgcacacactaaccttggagccgatgggattgagtgactggcacttgggaccacagagaaatgtcagagtgtttggttacagactcaaggaaacctctcattttagagtgctcatttggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaacaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaactggaaaaaaaa

SEQ ID NO: 2 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 3 - DNA, 1851 bp, Homo sapiens
atcatttgcatgtctagctggagaataaaaataatatatcatttgcatgtcttagtaatgaagtcatgggtgtcgtggcatgagatcagctggagggagaggagagattaaagtgaggagagagctacaaccaagtaagcaagtgtcagggctcaccaaccatgcaaggacagggcaggagaagaggaacctgcaaagacatattttgttccaaaatggcatcttacctttatggagtactctttgctgttggcctctgtgctccaatctactgtgtgtccccggccaatgcccccagtgcatacccccgcccttcctccacaaagagcacccctgcctcacaggtgtattccctcaacaccgactttgccttccgcctataccgcaggctggttttggagaccccgagtcagaacatcttcttctcccctgtgagtgtctccacttccctggccatgctctcccttggggcccactcagtcaccaagacccagattctccagggcctgggcttcaacctcacacacacaccagagtctgccatccaccagggcttccagcacctggttcactcactgactgttcccagcaaagacctgaccttgaagatgggaagtgccctcttcgtcaagaaggagctgcagctgcaggcaaatttcttgggcaatgtcaagaggctgtatgaagcagaagtcttttctacagatttctccaacccctccattgcccaggcgaggatcaacagccatgtgaaaaagaagacccaagggaaggttgtagacataatccaaggccttgaccttctgacggccatggttctggtgaaccacattttctttaaagccaagtgggagaagccctttcaccctgaatatacaagaaagaacttcccattcctggtgggcgagcaggtcactgtgcatgtccccatgatgcaccagaaagagcagttcgcttttggggtggatacagagctgaactgctttgtgctgcagatggattacaagggagatgccgtggccttctttgtcctccctagcaagggcaagatgaggcaactggaacaggccttgtcagccagaacactgagaaagtggagccactcactccagaaaaggtggatagaggtgttcatccccagattttccatttctgcctcctacaatctggaaaccatcctcccgaagatgggcatccaaaatgtctttgacaaaaatgctgatttttctggaattgcaaagagagactccctgcaggtttctaaagcaacccacaaggctgtgctggatgtcagtgaagagggcactgaggccacagcagctaccaccaccaagttcatagtccgatcgaaggatggcccctcttacttcactgtctccttcaataggaccttcctgatgatgattacaaataaagccacagacggtattctctttctagggaaagtggaaaatcccactaaatcctaggtgggaaatggcctgttaactgatggcacattgctaatgcacaagaaataacaaaccacatccctctttctgttctgagggtgcatttgaccccagtggagctggattcgctggcagggatgccacttccaaggctcaatcaccaaaccatcaacagggaccccagtcacaagccaacacccattaaccccagtcagtgcccttttccacaaattctcccaggtaactagcttcatgggatgttgctgggttaccatatttccattccttggggctcccaggaatggaaatacgccaacccaggttaggcacctctattgcagaattacaataacacattcaataaaactaaaatatgaattcatctgtcaaaaaaaaaaaa

SEQ ID NO: 4 - protein, 435 aa, Homo sapiens
MQGQGRRRGTCKDIFCSKMASYLYGVLFAVGLCAPIYCVSPANAPSAYPRPSSTKSTPASQVYSLNTDFAFRLYRRLVLETPSQNIFFSPVSVSTSLAMLSLGAHSVTKTQILQGLGFNLTHTPESAIHQGFQHLVHSLTVPSKDLTLKMGSALFVKKELQLQANFLGNVKRLYEAEVFSTDFSNPSIAQARINSHVKKKTQGKVVDIIQGLDLLTAMVLVNHIFFKAKWEKPFHPEYTRKNFPFLVGEQVTVHVPMMHQKEQFAFGVDTELNCFVLQMDYKGDAVAFFVLPSKGKMRQLEQALSARTLRKWSHSLQKRWIEVFIPRFSISASYNLETILPKMGIQNVFDKNADFSGIAKRDSLQVSKATHKAVLDVSEEGTEATAATTTKFIVRSKDGPSYFTVSFNRTFLMMITNKATDGILFLGKVENPTKS

SEQ ID NO: 5 - DNA, 2395 bp, Homo sapiens
atatagagcaggcgccgcgggtcgcagcacagtgcggagaccgcagccccggagcccgggccagggtccacctgtccccgcagcgccggctcgcgccctcctgccgcagccaccgagccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggcgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactcctgtacactgaatatttatatttcactatttttatttatatttttgtaattttaaataaaagtgatcaataaaatgtgatttttctgatgacaaaaaaaaaaaaaaaaaaaa

SEQ ID NO: 6 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 7 - DNA, 2377 bp, Homo sapiens
acccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaaaaaaaaaaaaaaaaaaaa

SEQ ID NO: 8 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 9 - DNA, 1542 bp, Homo sapiens
acaattactctacagctcagaacaccaactgctgaggctgccttgggaagaggatgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtgaatcccatcctggaagggaaggtgcatcgccatctacaggagcagaagaatggacttgctaaatgacctagcactattctctggcccgatttatcatatcccttttctcctccaaatatttctcctctcaccttttctctgggacttaagctgctatatcccctcagagctcacaaatgcctttacattctttccctgacctcctgattttttttttcttttctcaaatgttacctacaaagacatgcctggggtaagccacccggctacctaattcctcagtaacctccatctaaaatctccaaggaagcaataaattccttttatgagatctatgtcaaatttttccatctttcatccagggctgactgaaactatggctaataattggggtactcttatgtttcaatccaatttaacctcatttcccagatcatttttcatgtccagtaacacagaagccaccaagtacagtatagcctgataatatgttgatttcttagctgacattaatatttcttgcttccttgtgttcccacccttggcactgccacccacccctcaattcaggcaacaatgaaattaatggataccgtctgcccttggcccagaattgttatagcaaaaattttagaaccaaaaaataagtctgtactaatttcaatgtggcttttaaaagtatgacagagaaataagttaggataaaggaaatttgaatctca

SEQ ID NO: 10 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 11 - DNA, 1267 bp, Homo sapiens
acattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaa

SEQ ID NO: 12 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 13 - DNA, 1229 bp, Homo sapiens
cttgcctgcttctctggcccctggtcctgtcctgttctccagcatggtgtgtctgaagctccctggaggctcctgcatgacagcgctgacagtgacactgatggtgctgagctccccactggctttgtctggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaacaggattcctgagctgaaatgcagatgaccacattcaaggaagaactttctgccccggctttgcaggatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatggcagcgcctcatcttcaacttttgtgctcccctttgcctaaaccgtatggcctcccgtgcatctgtattcaccctgtatgacaaacacattacattattaaatgtttctcaaagatggagttaaaaaaaaaaaaaaaaaaaaaaaaaaaaaaaaaaaaaaaaaaaaaaaaaaaaaaaaaaaaaaaaaa

SEQ ID NO: 14 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 15 - DNA, 3743 bp, Homo sapiens
tctgccccttccccccgcccccgcccgcctcggctcccgcagcgctagtgtgtccgcctagttcagtgtgcgtggagattaggtccaagcgcccgcccagaggcaggcagtccgcgagcccagccgccgctgtcgccgccagtagcagccttcgccagcagcgccgcggcggaaccgggcgcaggggagcgagcccggccccgccagcccagcccagcccagccctactccctccccacgccagggcagcagccgttgctcagagagaaggtggaggaagaaatccagaccctagcacgcgcgcaccatcatggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgctttgccatccaggacatctccgtggaagagacttcagccaaggaagggctgctcctgtggtgtcagagaaagacagccccttacaaaaatgtcaacatccagaacttccacataagctggaaggatggcctcggcttctgtgctttgatccaccgacaccggcccgagctgattgactacgggaagctgcggaaggatgatccactcacaaatctgaatacggcttttgacgtggcagagaagtacctggacatccccaagatgctggatgccgaagacatcgttggaactgcccgaccggatgagaaagccatcatgacttacgtgtctagcttctaccacgccttctctggagcccagaaggcggagacagcagccaatcgcatctgcaaggtgttggccgtcaaccaggagaacgagcagcttatggaagactacgagaagctggccagtgatctgttggagtggatccgccgcacaatcccgtggctggagaaccgggtgcccgagaacaccatgcatgccatgcaacagaagctggaggacttccgggactaccggcgcctgcacaagccgcccaaggtgcaggagaagtgccagctggagatcaacttcaacacgctgcagaccaagctgcggctcagcaaccggcctgccttcatgccctctgagggcaggatggtctcggacatcaacaatgcctggggctgcctggagcaggtggagaagggctatgaggagtggttgctgaatgagatccggaggctggagcgactggaccacctggcagagaagttccggcagaaggcctccatccacgaggcctggactgacggcaaagaggccatgctgcgacagaaggactatgagaccgccaccctctcggagatcaaggccctgctcaagaagcatgaggccttcgagagtgacctggctgcccaccaggaccgtgtggagcagattgccgccatcgcacaggagctcaatgagctggactattatgactcacccagtgtcaacgcccgttgccaaaagatctgtgaccagtgggacaatctgggggccctaactcagaagcgaagggaagctctggagcggaccgagaaactgctggagaccattgaccagctgtacttggagtatgccaagcgggctgcacccttcaacaactggatggagggggccatggaggacctgcaggacaccttcattgtgcacaccattgaggagatccagggactgaccacagcccatgagcagttcaaggccaccctccctgatgccgacaaggagcgcctggccatcctgggcatccacaatgaggtgtccaagattgtccagacctaccacgtcaatatggcgggcaccaacccctacacaaccatcacgcctcaggagatcaatggcaaatgggaccacgtgcggcagctggtgcctcggagggaccaagctctgacggaggagcatgcccgacagcagcacaatgagaggctacgcaagcagtttggagcccaggccaatgtcatcgggccctggatccagaccaagatggaggagatcgggaggatctccattgagatgcatgggaccctggaggaccagctcagccacctgcggcagtatgagaagagcatcgtcaactacaagccaaagattgatcagctggagggcgaccaccagctcatccaggaggcgctcatcttcgacaacaagcacaccaactacaccatggagcacatccgtgtgggctgggagcagctgctcaccaccatcgccaggaccatcaatgaggtagagaaccagatcctgacccgggatgccaagggcatcagccaggagcagatgaatgagttccgggcctccttcaaccactttgaccgggatcactccggcacactgggtcccgaggagttcaaagcctgcctcatcagcttgggttatgatattggcaacgacccccagggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcatgtgtgtttgctagctcacttgtccatgaaaatattttatgatattaaagaaaatcttttgaaatggctgttttttaaggaagagaatttatgtggcttctcatttttaaatcccctcagaggtgtgactagtctctttatcagcacacacttaaaaaatttttaatattgtctattaaaaataggacaaacttggagagtatggacaactttgatattgcttggcacagatggtattaaaaaaaccacactcctatgacaaaaaaaaaaaaaaaaaaa

SEQ ID NO: 16 - protein, 892 aa, Homo sapiens
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRFAIQDISVEETSAKEGLLLWCQRKTAPYKNVNIQNFHISWKDGLGFCALIHRHRPELIDYGKLRKDDPLTNLNTAFDVAEKYLDIPKMLDAEDIVGTARPDEKAIMTYVSSFYHAFSGAQKAETAANRICKVLAVNQENEQLMEDYEKLASDLLEWIRRTIPWLENRVPENTMHAMQQKLEDFRDYRRLHKPPKVQEKCQLEINFNTLQTKLRLSNRPAFMPSEGRMVSDINNAWGCLEQVEKGYEEWLLNEIRRLERLDHLAEKFRQKASIHEAWTDGKEAMLRQKDYETATLSEIKALLKKHEAFESDLAAHQDRVEQIAAIAQELNELDYYDSPSVNARCQKICDQWDNLGALTQKRREALERTEKLLETIDQLYLEYAKRAAPFNNWMEGAMEDLQDTFIVHTIEEIQGLTTAHEQFKATLPDADKERLAILGIHNEVSKIVQTYHVNMAGTNPYTTITPQEINGKWDHVRQLVPRRDQALTEEHARQQHNERLRKQFGAQANVIGPWIQTKMEEIGRISIEMHGTLEDQLSHLRQYEKSIVNYKPKIDQLEGDHQLIQEALIFDNKHTNYTMEHIRVGWEQLLTTIARTINEVENQILTRDAKGISQEQMNEFRASFNHFDRDHSGTLGPEEFKACLISLGYDIGNDPQGEAEFARIMSIVDPNRLGVVTFQAFIDFMSRETADTDTADQVMASFKILAGDKNYITMDELRRELPPDQAEYCIARMAPYTGPDSVPGALDYMSFSTALYGESDL

SEQ ID NO: 17 - DNA, 5490 bp, Homo sapiens
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 18 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 19 - DNA, 2303 bp, Homo sapiens
gaattcgtccaaactgaggatcacaagtctccacattctgagtaggaggatgagggtctgagttaggatttgggtcctgcagggcttgctaaggaatcccctgatggcctaggattccacgcagagcacatctggtgtgagagagctcgctgcaagggtgaaggctccgccctatcagatagacaaccaggccaccaagaggcccagccctccaaaccctggatttgcaacatcctcaaagaacagcaacgggccttgagcagaattgagaaggaaatacccccacctgccctcagccgttaagtgggctttgctattcacaagggcctctgggtgtcctggcagagaggggagatggcacaggcaccaggtgctagggtgccagggcctcccgagaaggaacaggtgcaaagcaggcaattagcccagaaggtatccgtggggcaggcagcctagatctgatgggggaagccaccaggattacatcatctgctgtaacaactgctctgaaaagaagatatttttcaacctgaacttgcagtagctagtggagaggcaggaaaaaggaaatgaaaccagagacagagggaagctgagcgaaaatagaccttcccgagagaggaggaagcccggagagagacgcacggtcccctccccgcccctaggccgccgccccctctctgccctcggcggcgagcagcgcgccgcgacccgggccgaaggtgcgaggggctccgggcggccgggcgggcgcacaccatccccgcgggcggcgcggagccggcgacagcgcgcgagagggaccgggcggtggcggcggcgggaccgggatggaagggagcgcggtgactgtccttgagcgcggaggggcgagctcgccggcggagcgccggagcaagcggaggcgcaggagcggcggcgacggcggcggcggcggcggcgcccgagcacccgagggggtccgagccccggcagccggccagccccgcgccacaaagggagcgcccccgccgcccggcaccccgcctccctccccaatgtcctcggccatcgaaaggaagagcctggacccttcagaggaaccagtggatgaggtgctgcagatccccccatccctgctgacatgcggcggctgccagcagaacattggggaccgctacttcctgaaggccatcgaccagtactggcacgaggactgcctgagctgcgacctctgtggctgccggctgggtgaggtggggcggcgcctctactacaaactgggccggaagctctgccggagagactatctcaggctttttgggcaagacggtctctgcgcatcctgtgacaagcggattcgtgcctatgagatgacaatgcgggtgaaagacaaagtgtatcacctggaatgtttcaaatgcgccgcctgtcagaagcatttctgtgtaggtgacagatacctcctcatcaactctgacatagtgtgcgaacaggacatctacgagtggactaagatcaatgggatgatataggcccgagtccccgggcatctttggggaggtgttcactgaagacgccgtctccatggcatcttcgtcttcactcttaggcactttgggggtttgagggtggggtaagggatttcttaggggatggtagacctttattgggtatcaagacatagcatccaagtggcataattcaggggctgacacttcaaggtgacagaaggaccagcccttgagggagaacttatggccacagcccatccatagtaactgacatgattagcagaagaaaggaacatttaggggcaagcaggcgctgtgctatcatgatggaatttcatatctacagatagagagttgttgtgtacagacttgttgtgactttgacgcttgcgaactagagatgtgcaattgatttcttttcttcctggctttttaactcccctgtttcaatcactgtcctccacacaagggaaggacagaaaggagagtggccattctttttttcttggcccccttcccaaggccttaagctttggacccaaggaaaactgcatggagacgcatttcggttgagaatggaaaccacaacttttaaccaaacaattatttaaagcaatgctgatgaatcactgtttttagacaccttcattttgaggggaggagttccacagattgtttctatacaaatataaatcttaaaaagttgttcaactattttattatcctagattatatcaaagtatttgtcgtgtgtagaaaaaaaaacagctctgcaggcttaataaaaatgacagactgaaaaaaaaaaaa

SEQ ID NO: 20 - protein, 227 aa, Homo sapiens
MEGSAVTVLERGGASSPAERRSKRRRRSGGDGGGGGGARAPEGVRAPAAGQPRATKGAPPPPGTPPPSPMSSAIERKSLDPSEEPVDEVLQIPPSLLTCGGCQQNIGDRYFLKAIDQYWHEDCLSCDLCGCRLGEVGRRLYYKLGRKLCRRDYLRLFGQDGLCASCDKRIRAYEMTMRVKDKVYHLECFKCAACQKHFCVGDRYLLINSDIVCEQDIYEWTKINGMI

SEQ ID NO: 21 - DNA, 3591 bp, Homo sapiens
cgaccacccggcctcggccaataagcgccgccctctcgcccccgtgttactgggtagaagaaaacaaaaacaaacagagcgagaagggccagagactctccgaggcggcggcagagacagaagagcggggtcggggccggctgaccaggaacctgggcgagcagcggcgggggcccgagggattctgaaggaagatttccattaggtaatttgtttaatcagtgcaagcgaaattaagggaaaatggatgtagaaaatgagcagatactgaatgtaaaccctgcagatcctgataacttaagtgactctctcttttccggtgatgaagaaaatgctgggactgaggaaataaagaatgaaataaatggaaattggatttcagcatcctccattaacgaagctagaattaatgccaaggcaaaaaggcgactaaggaaaaactcatcccgggactctggcagaggcgattcggtcagcgacagtgggagtgacgcccttagaagtggattaactgtgccaaccagtccaaagggaaggttgctggataggcgatccagatctgggaaaggaaggggactaccaaagaaaggtggtgcaggaggcaaaggtgtctggggtacacctggacaggtgtatgatgtggaggaggtggatgtgaaagatcctaactatgatgatgaccaggagaactgtgtttatgaaactgtagttttgcctttggatgaaagggcatttgagaagactttaacaccaatcatacaggaatattttgagcatggagatactaatgaagttgcggaaatgttaagagatttaaatcttggtgaaatgaaaagtggagtaccagtgttggcagtatccttagcattggaggggaaggctagtcatagagagatgacatctaagcttctttctgacctttgtgggacagtaatgagcacaactgatgtggaaaaatcatttgataaattgttgaaagatctacctgaattagcactggatactcctagagcaccacagttggtgggccagtttattgctagagctgttggagatggaattttatgtaatacctatattgatagttacaaaggaactgtagattgtgtgcaggctagagctgctctggataaggctaccgtgcttctgagtatgtctaaaggtggaaagcgtaaagatagtgtgtggggctctggaggtgggcagcaatctgtcaatcaccttgttaaagagattgatatgctgctgaaagaatatttactctctggagacatatctgaagctgaacattgccttaaggaactggaagtacctcattttcaccatgagcttgtatatgaagctattataatggttttagagtcaactggagaaagtacatttaagatgattttggatttattaaagtccctttggaagtcttctaccattactgtagaccaaatgaaaagaggttatgagagaatttacaatgaaattccggacattaatctggatgtcccacattcatactctgtgctggagcggtttgtagaagaatgttttcaggctggaataatttccaaacaactcagagatctttgtccttcaaggggcagaaagcgttttgtaagcgaaggagatggaggtcgtcttaaaccagagagctactgaatataagaactcttgcagtcttagatgttataaaaatatatatctgaattgtaagagttgttagcacaagttttttttttttttttttttaagcacttgttttgggtacaaggcatttctgacattttataaacctacatttaaggggaatttttaaaggaaatgttttttcttttttttttgtttttcgagggggcaaggagggacagaaaagtaacctcttcttaagtggaatattctaataagctaccttttgtaagtgccatgtttattatctaatcattccaagttttgcattgatgtctgactgccactcctttctttcaaggacagtgttttttgtagtaaaatcactggtttatacaaagctttatttagggggtaaagttaagctgctaaaaccccatgttggctgctgctgttgagatactgtgctttgggagtaaaaaaagaaagttatttctttgtcttaaagaatttttaaaaaattagtcatgagacttattcatctttccagggaacatactgattggtcttaaaagactagacagttaagtaaaaggtggctggaacatctatttttctacaaaactggaaaaatgaacctggttctagaagaatgtacaccaaaataaaacatgtgaagcagtattgattctttattgggagtacatttttttaggtctcttaaactttaatttcacacagtaaattttgaatctcataaggaagcatatttgaacctagtcaatttaatcttagtgttcccttgaaaactttttttccctacaaaattttaagtgaaaaatacaatagtaaattaagattacactggggaaaaaaatgcaggtatcactttactccattgttatctgacctagagcttaattaagttttagaaatatgtaataccttccatcattccatcatccttaaattctgttaccaaataatggctaatgttacaaaaagttatactccagagacccaaagcttgacatttacctaatgtatgagaaaatattaccaattaacaataaagaatgatcatatttttaacctcttttacatagcctaataactcagcaaggcctcaacgtctgtgctaatttaaactgccaaatattgactgcagcaaacaagaattatattcagaatttatgagggtactgttaggagtatactgcttacaggtttagatatagtctgttagaattaaaaccaagtttagtgttcatatttacctcatgggctttatcaagcccatattacctcagcttatatatagttaccatttttaggtttttaattgtttgacacttggatgataaatgcagtcattttattctcaagtgcttaaaattaatgtaattaaaagcttagctgactacagaataggtgagggtttcttaaaaatgagatttaagggctgggcacggtggctcatgcctgtaatcccagcactttgggaggccgaggtgggcggatcacttgaggttgggagttcatgaccagcttgaccaacatgaagaaaccctgtctctattaaaaatacaaaagtagccaggcatggtggcgcatgcgtgtaatcccagctacttgggaggctgaggcaggagaattgcttgaacctgggaggcagaggttgcagtgagtcgagatggtgccattgctctcgtttgggcaacaagagtgaaactcttgtctcaaaaaaaaaaaaaaatgaggtttaagacagttttgtcattactggtgggatctggtcacacaagatagcattaaacgtgacatggcacataaaattggttaaaaaattttgttttttaattacgtaatgtaaaagcccaacaaacactttatgcaagattggaatgtatcttcaaattcagatttaataaacatgtaaagatcctctgtaaaaaaaaaaaaaaaaaaaaa

SEQ ID NO: 22 - protein, 469 aa, Homo sapiens
MDVENEQILNVNPADPDNLSDSLFSGDEENAGTEEIKNEINGNWISASSINEARINAKAKRRLRKNSSRDSGRGDSVSDSGSDALRSGLTVPTSPKGRLLDRRSRSGKGRGLPKKGGAGGKGVWGTPGQVYDVEEVDVKDPNYDDDQENCVYETVVLPLDERAFEKTLTPIIQEYFEHGDTNEVAEMLRDLNLGEMKSGVPVLAVSLALEGKASHREMTSKLLSDLCGTVMSTTDVEKSFDKLLKDLPELALDTPRAPQLVGQFIARAVGDGILCNTYIDSYKGTVDCVQARAALDKATVLLSMSKGGKRKDSVWGSGGGQQSVNHLVKEIDMLLKEYLLSGDISEAEHCLKELEVPHFHHELVYEAIIMVLESTGESTFKMILDLLKSLWKSSTITVDQMKRGYERIYNEIPDINLDVPHSYSVLERFVEECFQAGIISKQLRDLCPSRGRKRFVSEGDGGRLKPESY

SEQ ID NO: 23 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 24 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 25 - DNA, 3314 bp, Homo sapiens
tgcagagacacccttctcctttctgctgtctctgcacgggtggccagagccacacagccctttctttaagtcaggagttgccctgtcagagcacaaggcaagaaggaagtggtaaagggacggaggggaagccctgagaggactgagaggatgggaaattctctgctgagagaaaacaggcggcagcagaacactcaagagatgccttggaatgtgagaatgcaaagccccaaacagagaacatccagatgctgggatcaccatatcgctgaagggtgtttctgccttccatggaaaaaaatactcatttttgaaaagaggcaagattcccaaaacgaaaatgaaagaatgtcatctactcccatccaggacaatgttgaccagacctactcagaggagctgtgctataccctcatcaatcatcgggttctctgtacaaggccatcagggaactctgctgaagagtactatgagaatgttccctgcaaagctgagagacccagagagtccttgggaggaactgagactgagtattcacttctacatatgccttctacagaccccaggcatgcccgatccccagaagatgaatatgaacttctcatgcctcacagaatctcctctcactttctgcaacagccacgtccacttatggccccttctgagactcagttttcccatttatagtgaagtggctggactagcatttgtttagcaccaacaaataaaaggtgggatgggggatctgcctgaagcagggatgggacacaaagtccctccagcttatctcccacaacaaccctttccctgcagagcatggtttgtataccacaagccctcttagcacgcaaaagccaaaatctaaagatcaaccatttatcctgaacaacaccatttgagaaagaggtaaccatctttggttctacatggtttggagagtatagtggtaggaggggctccctgattcccctaaagctatgcacaccacaaggggctctgctcttctgtctgggatcttcttataaagtgttcccatgatcattctctaaagtcacaaggaagctttactcatcatactaagtgtgcccaagggggagttcactcattactgtgaccttccagctcagtccccacccatgggagcctgtgttgctcctctcactccatgtgtctaagtcatgtcttttacatagtgtcctttgacctgttggcccccatggtctggttagttatgtgagttgaatcaagaggctctaggccagatgtttacataattttaacctatatgattttatttttaactttgtatttctccctagaaatcttaataagacaattatgccatcagacaatgttaagaagaacgatccttggagatcccgtaatcccactacccttctttggctcagagaggataatttgcctaatgatacattaaagttagtggcaaaacttaatttggagcctgatttcctactgacttccaatttagtgctcccccagtatgctaaatagaaagccctctgcaatatattaaatgtatactaaatgtatatatttaataatgtcatgtataaaatatgaataaaatgtccacataggaaattaacacatatataccttctctgataagcactcctctatgtgtcctgattatttactttctcttatcttttccttagtgttcctcaaattatatctatcctctaaaccagggatcagcaaactataacccccaggccaaatctagcccactccctgtttttatagataaagatttgttggaacacagccacactcatttgtttacctactatctatgattgttcaaccatgggagagttgagtaattgtgacatggaccataggccctaaagagccaaaataaatatactttggcactttttggaaaaaaagtttgcagacacctgccctaaatgattatagtttgagagaaacttaacagaaatgtgtttggttactgatgcataagagcaagcaccaataaaatggcataaaaatattaaatcacaaatggataagtgagaccaaggcaatttagcaaattctactatttctgtattcacacctgtgtcttccacttgctgttttatttaactcacggctactgtcctaattttagggatgctaaaatagtttgttttaaaagaggtttgtttaagaaagcagtaggtacattatgtttactagacaggcccttggaccagctaagcgacagggaacctggtacattatgtttagttactgttttcttctcaaagttaaccaaagctaaataactttcctatgtaaaatgtgctgactcccaaaagaggagaaatgcaatggcttgagcttgcatgtgtcacaggtttgtggagccttcagccaggtgatggtcttcagctaaagagccagctgcatcttggtatctcttcctctgctaacttcctacttttagcaaaattctttcacttggaactctagctgatccttggccaggatcagatcctcaaaaggaaatattcagttagcagtggatattgtacaagatgtaacatggcttagaggaaagagtgtagacccagcgacagatcatcagtcactcaaatcctggttctctctcttcctccatctgtgactttgaacatgctacttggaggctcagcttcttcaccatcaacaggaggattacatctaatgcaggcaatcatctattacagatgccgaataaatgatatcaaagagcatagagaaacaaaaatcttttatgatgtacggaaaaaaaccatgctgatacagctggctattcagggtcacccagcagtcctccagcaaggtagaaacaccaaatcaatgagtttatgctagaatttttcacatggccactggaccattgtggaagtagactggttgaaatattcttagtatgacatagtaagttatatttgtttgaaccttatcctgtgaaagcattatctgtcccaaacagtatatgccttttatcagggacacattaaatataaatgtttaaccaaagaaagttgttcttataatgtaattataacagggatttataagagggaattataagagggattatgaaagggaattacttttgagcaaggggagccatttgggagtgcttcaagatacccaatatgatatacacaattatgatttgtcagtcaaaaataatattaagaacaaaaaaagagttaaaaaattctgatatgagtgtaatatacttaatatgtaggatgtctcaaatttccagtaaataaattttaatgtcccaaaaaaaaaaaaaaaaa

SEQ ID NO: 26 - protein, 178 aa, Homo sapiens
MGNSLLRENRRQQNTQEMPWNVRMQSPKQRTSRCWDHHIAEGCFCLPWKKILIFEKRQDSQNENERMSSTPIQDNVDQTYSEELCYTLINHRVLCTRPSGNSAEEYYENVPCKAERPRESLGGTETEYSLLHMPSTDPRHARSPEDEYELLMPHRISSHFLQQPRPLMAPSETQFSHL

SEQ ID NO: 27 - DNA, 1157 bp, Homo sapiens
gctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttc

SEQ ID NO: 28 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 29 - DNA, 3968 bp, Homo sapiens
ataagtggagtgtgctggggtgtgtaaagtagtatggaggcagcggtagcccagtgtctgagtggttgccgggtctccatggagaagcggctcgccagtgtcccaggctgctgagctctcgccgcccgagaccccgcggcgcggccgcagggccatgctagccttgcgcgtggcgcgcggctcgtggggggccctgcgcggcgccgcttgggctccgggaacgcggccgagtaagcgacgcgcctgctgggccctgctgccgcccgtgccctgctgcttgggctgcctggccgaacgctggaggctgcgtccggccgctcttggcttgcggctgcccgggatcggccagcggaaccactgttcgggcgcggggaaggcggctcccaggccagcggccggagcgggcgccgctgccgaagccccgggcggccagtggggcccggcgagcacccccagcctgtatgaaaacccatggacaatcccgaatatgttgtcaatgacgagaattggcttggccccagttctgggctatttgattattgaagaagattttaatattgcactaggagtttttgctttagctggactaacagatttgttggatggatttattgctcgaaactgggccaatcaaagatcagctttgggaagtgctcttgatccacttgctgataaaatacttatcagtatcttatatgttagcttgacctatgcagatcttattccagttccacttacttacatgatcatttcgagagatgtaatgttgattgctgctgttttttatgtcagataccgaactcttccaacaccacgaacacttgccaagtatttcaatccttgctatgccactgctaggttaaaaccaacattcatcagcaaggtgaatacagcagtccagttaatcttggtggcagcttctttggcagctccagttttcaactatgctgacagcatttatcttcagatactatggtgttttacagctttcaccacagctgcatcagcttatagttactatcattatggccggaagactgttcaggtgataaaagactgatgaaagtcatccctcactgttagtaaggaagcagtatacatcaatgggaacagggcccatggaaatgtacaggagtttccctattttggtgttcagcttgaaaaaggacttgtcagaatcaactgtgtcatcaaaatttaagtaatgtgcattgaaaataaggttgatcatgggaatatgcagaatttccaatgtatttttaaatacaaataaaattgtaatttagaatttttaatcttaggtttcttgattaatttataagagatcaattattgtcagtcttttttgtatgttttttaaaaacatagtccagagcatgggcagaattgacacctctcttttaagtgaaatttggattgctcacaaagcactaggaaatgtcatggggttcaaatatatatcctacacaactgggcaatacatttttgtttgatttttaggtctgtgtatacattaacagttcatgtaattaatacctgatcatttgggataatgaaagtgaagttagttgtagatgaagtaaagttataaaagagattaaaaatgcggtaactttttaagataataatcatacagaaggtatgaagttcattttcggtagtcttccaacctctcaggtgcctaataatttatgtttgaggataacaggtaacaaagatagtcgagataggagaacgtgtctattagtctttgcatctaaaaggcagtgagttacgttcctgccttccactgtgtttctgacatagcaatgtttgtttgatattggaacctggattcatattttatgtaaataatatcaagctgtatatttttcaaaggttttttaaactttggagactctttcttttgttaagcagttaaaggaataaaagagctggaaaaaaaattgtaccttcaactcaggttgttccatataacatacgtattctctgctgttacgtaagttttccgattcacagagtccattcatgtacatcacttacacttaaattgtaaaaataattagtctgaccatctgactttaaaagactgttgctacacgtacatcatgtttaggagaatgtgggatatggggaaggggagaagaagacagtccagtggtgactagttgctgctgcttaacctatggcctattgctgcccattgtccctggttttaatccaagcctattatggaatactgcagtgagattatccattctcagcctgttgagctgcattaaattagaatgggaggctgtaagtaaaactcatgcctcttggccaggcattctgcaccagcgtgtccctctgtggcttgctggtcctggttgttctcagactggcagctgagctgcgcttggcttgaagcatactgaagcctgcagccagggacaccgccattgaatcagatggagggggatgaaaaagcagagaagggggcatgtggctggctattcttgctctctggagaacagcttcattgttaggagtagttaggctgaaagtggacttcccactgctgagccacccaagaggcacactgagcattagtgacgggtgagccacataagaataatgtttaaaaaagagaggacttgaagtagctatgcgaaaatttagaactttgcactttgttaacgcttatttatggtttagtgttgatgccatttggagtgacctatgggttggacccatatctttttattattaaggtctcaatctggctctggttaataaacgtttcccgcttcatcccttaggaaaatggtccaagtctcctcaggcttttggatactgaattgttataaaacacatcaattctgccagtttataaaacactggaattgtattagacatgtctgagtatgtacttgtttatagactcaaaacatgatgaaaattcatttcattgtcttcactgaatagacttaaccttgacagtaagcttaaagcattagtgtcatttaattacatgtctgaggaatttatgctaactagattttgggcttttgcagcgaattttagtcggggcctcattcctgagcccaagtgaccctttcacctcagcttcccaagtagctgggattacaggtgcacaccaactgtgctttgcagttttgtttgtgtgtgtgtgtgtgtgtgtgtgtgtgtgtgtgtgtgtgtgtattttgttttttttacaagcccacaacatcatagctttgcagtttttaaccaggagctggtggagatctttgaaatcatttaggccaattcttttagttacatttatgatgaaactagggatctaccttaaagttttgggaagctctttcaaaacttcagacacctagaaatatatatagtgcagttcagttttagtttctgcagcatggtttggaaacaaaagatacataggatttaaactcgatttgttttcttcttacagtagaagtaaatgctcttgtggcttgaatcgcatctcccaagaaagatacgttcacgttcaaacccccagtactgtacatgtgaccttatttggaaatagggtctttttagatgcagtcaagatagaattagatcatactggattacagtgggccccaataactaggtttttttgttaaaaaaaaaaaaaaaggaaatgtgaacagagagagaatgccgtgagatgatggagggatagattggaatgatgtctgcgagctaaggaatactgaggattgctgccaaccatccgaagctgggagaggagatggaacaactccctcggagcctccaagaggaaccaaccccactgatgctttgatttgggatttccggcctgaactgtaggaaaataaatttctgttcttttaagccatttttaccagtttgtggtaacttgttacagcagctgcaggaaattaataaatgccaatgttgaaaattaaaaaaaaaaaaaaaaa

SEQ ID NO: 30 - protein, 301 aa, Homo sapiens
MLALRVARGSWGALRGAAWAPGTRPSKRRACWALLPPVPCCLGCLAERWRLRPAALGLRLPGIGQRNHCSGAGKAAPRPAAGAGAAAEAPGGQWGPASTPSLYENPWTIPNMLSMTRIGLAPVLGYLIIEEDFNIALGVFALAGLTDLLDGFIARNWANQRSALGSALDPLADKILISILYVSLTYADLIPVPLTYMIISRDVMLIAAVFYVRYRTLPTPRTLAKYFNPCYATARLKPTFISKVNTAVQLILVAASLAAPVFNYADSIYLQILWCFTAFTTAASAYSYYHYGRKTVQVIKD

SEQ ID NO: 31 - DNA, 904 bp, Homo sapiens
agtttgaagaaggctcttacagcatggccgccggtactgcagctgccttagcgtttttgagtcaggagagccgaacgcgggccgggggtgtcgggggcctacgggtcccggccccggtcactatggacagttttttcttcggctgtgagctctccggccacacccgctccttcacctttaaggtagaggaagaggatgatgcggagcacgtgctggcactaaccatgctctgcctcaccgagggagccaaagacgagtgtaatgtggtagaagttgtggcccggaaccatgaccatcaggagatcgcagtccctgtggccaacctcaagctgtcctgccaacccatgctcagtctggatgacttccagctccaaccacctgtaaccttccgcctgaagtcgggctctggccctgtgcggatcactgggcggcaccagattgttacgatgagcaatgatgtttctgaggaggagagcgaggaagaggaagaggacagtgatgaggaagaagttgagctgtgccccatccttcctgccaaaaagcaggggggcaggccctagccctcctaggtcagctccatgtgccatgcaccgccatgcaccctgttccctgacaagtttcaacaattgtaaatatttcttccttgaagaggagagcttgggtgggggttgggtgggagggacttgggtctttggtgctaggagagggcctgtgctccacacagccgtggttttctgattttcaccatgcccggggcctcccttcccacctgcctgtgagaattggaggttagtgcctgaagctcagagctacacatttttaatagtttttacatttttggataaaggttgaaataaagtggtgtggagtttttgcaaaaaaaaaaaaaaaaaaaaaaaaaaaa

SEQ ID NO: 32 - protein, 178 aa, Homo sapiens
MAAGTAAALAFLSQESRTRAGGVGGLRVPAPVTMDSFFFGCELSGHTRSFTFKVEEEDDAEHVLALTMLCLTEGAKDECNVVEVVARNHDHQEIAVPVANLKLSCQPMLSLDDFQLQPPVTFRLKSGSGPVRITGRHQIVTMSNDVSEEESEEEEEDSDEEEVELCPILPAKKQGGRP

SEQ ID NO: 33 - DNA, 3105 bp, Homo sapiens
caactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcaatgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttcccttgtagaaagtgaggctcagattaaattttagaatattttctaaatgtctttttcacaatcatgtactgggaaggcaatttcatactaaactgattaaataatacatttataatctacaactgtttgcacttacagctttttttgtaaatataaactataatttattgtctattttatatctgttttgctgtaacattgaaggaaagaccagacttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggca

SEQ ID NO: 34 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 35 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 36 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 37 - DNA, 6492 bp, Homo sapiens
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 38 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 39 - DNA, 776 bp, Homo sapiens
aaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctagagctgagtgcctgaggggtccagaagcttcgaggcccagcgacctcggtgggccagtggggaggagcaggagcctgagccttgggaaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagcacgctcttttaaaaggt

SEQ ID NO: 40 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 41 - DNA, 6480 bp, Homo sapiens
agagcgagcaggggagagcgagaccagttttaaggggaggaccggtgcgagtgaggcagccccgaggctctgctcgcccaccacccaatcctcgcctcccttctgctccaccttctctctctgccctcacctctcccccgaaaaccccctatttagccaaaggaaggaggtcaggggaacgctctcccctccccttccaaaaaacaaaaacagaaaaacccttttccaggccggggaaagcaggagggagaggggccgccgggctggccatggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtgaggatgccagttgggccgaaagagagagacgcgtccataatctggtctcttcttctttctggttgtttttgttctttgtgttttagggtgaaacttaaaaaaaaaattctgcccccacctagatcatatttaaagatcttttagaagtgagagaaaaaggtcctacgaaaacggaataataaaaagcatttggtgcctatttgaagtacagcataagggaatcccttgtatatgcgaacagttattgtttgattatgtaaaagtaatagtaaaatgcttacaggaaaacctgcagagtagttagagaatatgtatgcctgcaatatgggaacaaattagaggagacttttttttttcatgttatgagctagcacatacacccccttgtagtataatttcaaggaactgtgtacgccatttatggcatgattagattgcaaagcaatgaactcaagaaggaattgaaataaggagggacatgatggggaaggagtacaaaacaatctctcaacatgattgaaccatttgggatggagaagcacctttgctctcagccacctgttactaagtcaggagtgtagttggatctctacattaatgtcctcttgctgtctacagtagctgctacctaaaaaaagatgttttattttgccagttggacacaggtgattggctcctgggtttcatgttctgtgacatcctgcttcttcttccaaatgcagttcattgcagacaccaccatattgctatctaatggggaaatgtagctatgggccataaccaaaactcacatgaaacggaggcagatggagaccaagggtgggatccagaatggagtcttttctgttattgtatttaaaagggtaatgtggccttggcatttcttcttagaaaaaaactaatttttggtgctgattggcatgtctggttcacagtttagcattgttataaaccattccattcgaaaagcactttgaaaaattgttcccgagcgatagatgggatggtttatgcaagtcatgctgaatactcctcccctcttctcttttgccccctcccttcctgcccccagtctgggttactcttcgcttctggtatctggcgttctttggtacacagttctggtgttcctaccaggactcaagagacaccccttcctgctgacattcccatcacaacattcctcagacaagcctgtaaactaaaatctgttaccattctgatggcacagaaggatcttaattcccatctctatacttctcctttggacatggaaagaaaagttattgctggtgcaaagatagatggctgaacatcagggtgtggcattttgttcccttttccgttttttttttttttattgttgttgttaattttattgcaaagttgtattcagcgtacttgaatttttcttcctctccacttcttagaggcattcagttagcaaagaggttggagcaacaacttttttttttttttttgcacaattgtaattgacaggtaatgaagctatttgttaaaatatttgcctttttaagtaaaaaagaaaaatcagaacagggctatttgaagaattattttatacacagattctgccttgtttcatagtatgagggttgaagacggaaaacaatctaagggtctctcatttttttaattttgttttgttcagtttggtttttttttttttttgcgctgctaagaagctaaagtcatccatccttattcacgttgacagtacctagctgtaatgtttcacagagtgtgctgctattttataaacatttttataatatattattttactgcttaaattccaagtcctgaagtagatggttgagatatgagttcttcgtactggaaaagcccttccgtagtttgttttcttctggtagcatattcatggttgtttttttttttcttttttggttttttggttttttttttttcctctgatcacattcttcaaagacggagtattctttacctcaggtttactggacaaaatcaataactacaaaaggcaatgattcacgcttttgttttcataatacctcacaaccgtacagtttctgcttgggagcccattcgcatgaggaatacagaagcagtgtgagcagggctgactccctctcaggtggaaggcagggcggtctcactcccagggacctttttggtcatggaggccatcgggctcccagttagaccctggtatcctcatcatgatggaaaaaatacattgaaccaagggatcctccctccccttcaaggcagacgttcagtacaaacatttatgcggtaggctcagatgtcgtaatttgcacttaggtaccaggtgtcaggaaacagactaaaaagaattccaccaggctgtttggagatcctcatcttggagctttttcaaaagcggggcttcatctgcaaagggccctttcatcttgaagtttttcccctccgtctttcccctcccctggcatggacaccttgtgtttaggatcatctctgcaggtttcctaggtctgaatctgcgagtagatgaacctgcagcaagcagcgtttatggtgcttccttctccctcctctgtctcaaactgcgcaggcaagcactatgcaagcccaggccctctgctgagcggtactaaacggtcgggttttcaatcacactgaattggcaggataagaaaaataggtcagataagtatgggatgatagttgaagggaggtgaagaggctgcttctctacagaggtgaaattccagatgagtcagtctcttgggaagtgtgtttagaagggttcaggactttgtgagttagcatgaccctaaaattctaggggatttctggtgggacaatgggtggtgaattttgaagttttggagagggaagtggagcagccagcaagtaagctagccagagttttctcaagagccagctttgctcagcacactctcctgggccccaaggagtcccacggaatggggaaagtgggaaccctggagttcttgggaatcttggagcctaaagagaaaccgaggtgcaaattcatttcatggtgactgacccttgagcttaaacagaagcagcaaatgaaagaaccggacaaataaggaagggcacaagcctacccgactctatttacagtctgtaactttccactcttcctgtagtcccgaggcccctgggtccttctagcttttctctttcccatccttggggccttgtgtgatgatgggtgtggggctgccgatgggaaagtcgggggttgttaggcttttctgcctgctcctgcttaaacacaagaaggaatcctggattttgccctctccttagctcttagtctctttggtaggagttttgttccagaggagctctcccccttggatttgaacttgctctttttgttgttgttgttctttctcttctttttcttacctcccactaaaggggttccaaattatcctggtctttttctaccttgttgtgtttctatctcgtctttacttccatctgtttgtttttttctccatcagtgggggccgagttgttcccccagcctgccaaattttgatccttcccctcttttggccaaatcctagggggaagaaatcctagtatgccaaaaatatatgctaagcataattaaactccatgcgggtccataacagccaagaagcctgcaggagaaagccaagggcagttccctccgcagaacaccccatgcgtgctgagaggcgagctccttgaagaaggggctgttcttccaggaggccttattttgaactgcctcaggaccccactggagagcacagcatgccttactactgggtcatccttggtctatgtgctctgtactggaggctctgttctgcctcttatcagccaggtcaggggcacacatggcttaagtgacaaagccagaggagaagacaaccctgacagcatcacgctgcatcccattgctagcaggattggcaactcttcagacggagctgcgcttccctgcagtctagcacctctagggcctctccagactgtgccctgggagctctgggactgaaaggttaagaacataaggcaggatcagatgactctctccaagagggcaggggaattttctctccatgggccacaggggacagggctgggagaagaaatagacttgcaccttatgtcatgtaaataattgattttctagttcaagaagataatattggtagtgtgggaattggaggtaggaaggggaggaagtctgagtaagccagttggcttctaagccaaaaggattcctctttgtttatctctgagacagtccaaccttgagaatagctttaaaagggaaattaatgctgagatgataaagtccccttaagccaacaaaccctctgtagctatagaatgagtgcaggtttctattggtgtggactcagagcaatttacaagagctgttcatgcagccatccatttgtgcaaaatagggtaagaagattcaagaggatatttattacttcctcataccacatggcttttgatgattctggattctaaacaacccagaatggtcatttcaggcacaacgatactacattcgtgtgtgtctgcttttaaacttggctgggctatcagaccctattctcggctcaggttttgagaagccatcagcaaatgtgtacgtgcatgctgtagctgcagcctgcatcccttcgcctgcagcctactttggggaaataaagtgccttactgactgtagccattacagtatccaatgtcttttgacaggtgcctgtccttgaaaaacaaagtttctatttttatttttaattggtttagttcttaactgctggccaactcttacatccccagcaaatcatcgggccattggattttttccattatgttcatcacccttatatcatgtacctcagatctctctctctctcctctctctcagttatatagtttcttgtcttggactttttttttcttttctttttctttttttttttgctttaaaacaagtgtgatgccatatcaagtccatgttattctctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcagctgccttttaagaggtcttatctgttcagtgttaagtgatttaaaaaaataataacctgttttctgactagtttaaagatggatttgaaaatggttttgaatgcaattaggttatgctatttggacaataaactcaccttgacct

SEQ ID NO: 42 - protein, 289 aa, Homo sapiens
MELLCHEVDPVRRAVRDRNLLRDDRVLQNLLTIEERYLPQCSYFKCVQKDIQPYMRRMVATWMLEVCEEQKCEEEVFPLAMNYLDRFLAGVPTPKSHLQLLGAVCMFLASKLKETSPLTAEKLCIYTDNSIKPQELLEWELVVLGKLKWNLAAVTPHDFIEHILRKLPQQREKLSLIRKHAQTFIALCATDFKFAMYPPSMIATGSVGAAICGLQQDEEVSSLTCDALTELLAKITNTDVDCLKACQEQIEAVLLNSLQQYRQDQRDGSKSEDELDQASTPTDVRDIDL

SEQ ID NO: 43 - DNA, 7118 bp, Homo sapiens
tagcaacacagagcagctctgagaggggaggaggaggagaagaaggaagacagggaagtgggagagacagagggagagtgtgcatacagcagctgtcggcatccctgtctcagggacttctttgctgattcacagaggcagcccagccccggcctcccagcttacagctgccgcctgcttctcagacctgacggaatcagaagctacataatattcgccgtgtgagtggctatcgtaatagataacatttgaatatctactaaatgccagaaactatgaattaactcctctcaacaaagctaagaggtgcttaatgatcaatagttggtgcactgcctatcctcatcccccccatgagccttggtttttatgcaacctatggtcatgcagggatgcccttacaccctcccacgatgtcatgactggcaggcagctgaccagttccatcatagcagcagcctccgaagcacctgcccccaccctcaggttagagctgctgtcaccagccctgcacctcctcaagatggtgctggggttccctgcctaagcctaaagctgttgaacgggtctgttggtgcctctggacccctggaaccaccagccatgaatctgtgttggaatgaaataaaaaagaagtctcacaacctccgcgctcgcctagaggccttctcagaccacagtggaaagcttcagctccctcttcaagagattattgactggctcagccaaaaggatgaggagttgtcagctcagctgcccctacagggggatgtggccctggtgcaacaggagaaggagacacatgcggcctttatggaagaagtcaagtctcggggcccctacatctattctgtgctggagtcagctcaggccttcctgtcccagcacccatttgaggagttagaggagcctcattctgagagcaaagatacctccccgaaacagcggatccagaatctcagccgctttgtatggaagcaggcgacggtggccagtgaactgtgggagaagttgacagcccgctgtgtggaccagcaccgtcacattgagcggactctggagcagctcttggagattcaaggggcaatggaggaactaagcactactctgagccaagctgagggagtccgagccacttgggagcccattggggatctcttcattgattcactcccagagcacatccaggctattaagctgttcaaagaagaattctcccccatgaaagatggagtaaagttggtgaatgatctggcccaccaacttgccatttctgatgtgcacttgtcaatggagaattcccaggccctggaacagatcaacgtccgatggaaacaactacaggcgtcagttagtgagaggcttaagcagctccaggatgcccaccgggactttgggcctgggtcacagcactttctctcctcctctgtccaggttccctgggaaagagcaatttcacccaataaagttccctactacatcaaccaccaggctcagaccacatgctgggaccatcccaagatgacagagttataccaaaccctagctgatctgaacaacattaagttctcagcttatcgcactgccatgaaactccgcagagtccagaaagccctgcgcttggacctggtaactttaaccacagccctggaaatcttcaatgagcatgatctgcaggccagtgagcacgtgatggatgtggtagaggtcattcactgcctgactgccttatatgaacgtttggaggaggaaagaggcatcctggtcaacgtgccactctgtgtggacatgagcctcaattggctcctcaatgtttttgatagtggtcgcagcggaaagatgcgggcattgtcttttaagactggcattgcatgcttgtgtggcacggaagtgaaggaaaaacttcagtacctcttcagccaagtggccaactcaggcagccagtgtgaccagcgccaccttggtgtcctgcttcatgaggccattcaggtgccccgtcagctgggtgaagtggcagcctttgggggcagcaatgtggagcccagtgtccgtagttgcttccgttttagcaccgggaagccagtcattgaagcatcccagttcctggagtgggtcaacctggagccccagtccatggtgtggctggctgttctgcatcgggtcaccattgctgagcaagtgaagcatcagaccaagtgctctatctgtaggcagtgccccatcaaggggttcaggtaccggagtctgaagcaattcaacgttgacatctgccagacctgcttcttgacaggcagggccagcaaaggcaataagctgcactaccccatcatggagtattacacaccgaccacatccagtgagaacatgagggactttgccacaaccttaaagaacaaattccgctccaagcattatttcagcaaacaccctcagcgaggttatctgcctgtgcaatcagtgctggaggctgactacagtgagacgccggcttcttccccgatgtggccacacgccgacacacactcccgaattgagcattttgccagcaggcttgctgagatggaaagtcaaaattgctccttctttaatgacagcttgtccccagatgacagcatagacgaggaccagtacctgctgcggcactccagccccatcacagaccgggagccagcctttggacagcaggctccatgcagtgtggccacagaaagcaaaggggagctacagaagatcctggcccacttggaagatgagaaccggattctccagggagagctgaggcgcctgaagtggcagcatgaggaggcagctgaggcacccagtctggctgacggctccactgaggcagcaacagaccaccgcaatgaggagcttctggccgaggcccgtatccttcggcaacataagagccgcctggagacgcgcatgcagatcctcgaagatcacaacaagcagctagagtcccagctgcagcgtctgagggagcttctcctgcagccacccaccgaatcagatggcagtggctctgcaggctcgtccctagcttcctctccacagcagtcagaaggcagtcacccccgggagaagggacagactactccagataccgaggctgcagatgatgtggggtcaaagagccaggatgtcagcctgtgcttggaggacatcatggagaaactccgtcatgccttccccagtgtgcgaagttctgatgtgactgccaacaccctgctggcctcttgatggagccagatccccatcctatagttcatagtcctctcctggttccggtcaaagcctttcctcagccttcacccaacctttccagtttccactggccccacattcctcaactagtattatttgggctctgggcagcagcagggatctggtggtatgtgaggtgcatgcgggcagtgatgggagaaggggaggcatgattcttctgaccctagaaatgttccctttaatcttcaagttcgagatcagccctttaagtacctttctgttgcagcccaggcaaatatcacttggccattcagatggggagcagagaagctgccttgcagagctaagcggtacatgttggcccctcttccttctccgtggaattatggaaagagagaagcctaattaccccaaggttccatccagcattatgaatccacagggtttacccctactctctggcgcaactcagggaggcaaaagggtatccccgagacaccttccccaccccaagtgcccctgaaaaagtctgggcagtgtgtgttctattacttcagctccaggacagattgctagccatcctaatgctacctaggtattggtttctcctcaaatatcttcattttctttagcctcagtccctgacagaccttccttaaggggagaacagttcattctggttcttctacgttatatttggaagcattaaatccatacttatgggtttagattttttttttgccccacatcttcctatttcttaaagtcctcagcattttgagagacccactgagttgcgagttgctctggaaacctggctgaagccaccctttgctcctctgagtctgtcctcacagacctgaggacacagtggcagcttgctgagcaagggagtgttgctttccttgggtaaggaagaggaagcccacctcaagtctggagtagtatgactgccatccaacacccttccaggctgtgacccttctagctctcaggctgaatcaggtgaggtggctgattgtccccagctaatcataaggcatttgcaactggctaccgtgtgtagtggcccctccataggagccacaggccacaaatcaggaagctggccactttccacccagacaacaacagcaaggccttcctgtgcctaaaattgttatttctaactctggaaggtgaatccaatcctcaaactgaactagctcttcctaggcgacatataccatgatctctttatccagaaactgttcagaatagatagtaggggtgggggagtgggcttgggaatgttgaatagattcagagcccacttaggcagctttctcccaagaggagctttataacctagagccctgcagttgacttcatttgcctaagtttatatacatacatatctattctacatatctactgtacatcagtgctctcctggcctcactcacacattaaattctaatacctcttgaagtattaatgttctgccttgctctttgttagccccagcctctggttggaaagatagatgtatcctggctgttcccctaggatatctctctccattgatgtttggggtgggctgtcctcaagaccaaagcaacagaaacctgtggtcacagagaatctaacaggaagagtcactgttctcttcactcattccaaatcttgttcccttgagacctgggccttgggtgaattttcctgggctcccctaaatggttcaggtgccaaaatagagcaaacccaagctctgcccaccccatgcagaaaaggacaaacaggcctttattagcctttgccagctaagacacagagaacattctctggtcccttccagctcttatggtctcactcagggctaaagaaagcaacagttgccaccacaatcaggagggagtgacggttggggtgttgtattataatccagcagtgggccagtgggtagaactgctaggaacctagtttgatctgctgcaggggaggcagggcagtggcttgggtccagaagcttagggttgcctcctccaccactaagtcacaggtccagggtgtcagacttcacagcataacatctgccttagtgttcatgaggtctctgggcaggagaactgggtatctggatagatggttgagcaggtggtaggcgggcatcaatcatagaacctgggcatcagtcagagtgggagcatatttgtagaagcctagattgtgggcgggaatgtgggacggagggctcaaggactttggaggcatatgcctgcctaagccttaggcgggtacatgttatggagcaattttttaccaggaaagtcctggaaaatcttttctgttatccccaccccacctgagagctcttgggctgctgagggcctgtggcaactgcagtgggccaggcatggtagaaaagatgtagatcttgttatgtaggatttgcgttgtaggtggactgaagcagcaggtcaagagttgaattctcactaaggggtcactgacattttgtggtcctgtgtttaccttaatgcctaaggtgagtgtattgttcaagggtccagggacagtcactgcagtatgtgtggggaattttcccacagtaaggtaaatggatccttgggtactatgaatccaacttggggttggatttaccaaaatgtgttgtatgttgttagacatcttgttgtgtattagtcttctaaggatattgtgcatggcactgtgtagtttatagccctacactggaaggatggtgaagggagttgctagattgatagcctaaagaaaaccttctgactgtgggattagcatcaatgataaggaatgtgagcatttgctggggttgttgtcaaatagtatcagagaacagttaggactgagaggtgatttcttcttgacagaatttgatagaggccaaaaagccattacgaggatacctctgcttccagtgacagcagagtggattcacattgattgggcattgtcacggaaggctgactagctggaggacatcaggctgtgctatgcttccctagggaggacatgtgcccttccctccagattagaaaggctagaatcaagagtttcaagtgctcattttcttcaaggtccaaggttgggaactatttccctacagagaaatctgtttgagtttctggtgctccaggaaatgggttggcaggacacagaaatgagaaaccctgaggccaggaagaggccaatcctaggagatgttttccctccagattcattttaaggaatatctgtgcttcctgatgtgtgtgcttgagatttgggctctatatccagcatttgaactcacattctcagtggcagagtcatggtctgtggcaccatttcaaggccaaaaatcccatccaatttcccttcacatagatgacagaaaagcaagaaagtagctgccttaactcgaacagttctgggaaaaaaactgggggccttgtcctgaggctgtggagacttaattcttcagctgggtggttcccgtaggaacaaggaacactttagctacttcttactcttttaacaccaatggctgtgggagaaggaaaggctcaaggccagggctcagctcgcagagtccatcttctcccacagttgccacatttgagagggggcctgctgaatgcccttgctgtcccctatggcagtattgaggcctgaccgaagtctggtatgctgcttgctggctacaagaactggtcacaggatgaaaattggggaaaggatttgggggaggtgacaagggtggcatggaagtctaggggacaaagggacagggttgggaacaaattggttaactttgattgccactcactgtggtgctttccccctttcccttgaattctgctttgaaaagaataaatttgtacttgttt

SEQ ID NO: 44 - protein, 957 aa, Homo sapiens
MQPMVMQGCPYTLPRCHDWQAADQFHHSSSLRSTCPHPQVRAAVTSPAPPQDGAGVPCLSLKLLNGSVGASGPLEPPAMNLCWNEIKKKSHNLRARLEAFSDHSGKLQLPLQEIIDWLSQKDEELSAQLPLQGDVALVQQEKETHAAFMEEVKSRGPYIYSVLESAQAFLSQHPFEELEEPHSESKDTSPKQRIQNLSRFVWKQATVASELWEKLTARCVDQHRHIERTLEQLLEIQGAMEELSTTLSQAEGVRATWEPIGDLFIDSLPEHIQAIKLFKEEFSPMKDGVKLVNDLAHQLAISDVHLSMENSQALEQINVRWKQLQASVSERLKQLQDAHRDFGPGSQHFLSSSVQVPWERAISPNKVPYYINHQAQTTCWDHPKMTELYQTLADLNNIKFSAYRTAMKLRRVQKALRLDLVTLTTALEIFNEHDLQASEHVMDVVEVIHCLTALYERLEEERGILVNVPLCVDMSLNWLLNVFDSGRSGKMRALSFKTGIACLCGTEVKEKLQYLFSQVANSGSQCDQRHLGVLLHEAIQVPRQLGEVAAFGGSNVEPSVRSCFRFSTGKPVIEASQFLEWVNLEPQSMVWLAVLHRVTIAEQVKHQTKCSICRQCPIKGFRYRSLKQFNVDICQTCFLTGRASKGNKLHYPIMEYYTPTTSSENMRDFATTLKNKFRSKHYFSKHPQRGYLPVQSVLEADYSETPASSPMWPHADTHSRIEHFASRLAEMESQNCSFFNDSLSPDDSIDEDQYLLRHSSPITDREPAFGQQAPCSVATESKGELQKILAHLEDENRILQGELRRLKWQHEEAAEAPSLADGSTEAATDHRNEELLAEARILRQHKSRLETRMQILEDHNKQLESQLQRLRELLLQPPTESDGSGSAGSSLASSPQQSEGSHPREKGQTTPDTEAADDVGSKSQDVSLCLEDIMEKLRHAFPSVRSSDVTANTLLAS

SEQ ID NO: 45 - DNA, 8014 bp, Homo sapiens
agctggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaagtagatgtgtagatctccgtccttcatttctgtcattcaagctcaacggctattgtggtgacatttttatgtttttcattgccaagttgcatccatgtttgattttctgatgagactagagtgacagtgtttcagaacccaaatgtcctcaggtagtttggagcatctctatgagatgggattatgcagatggcctatggaaaatgcagctgcataattaacacattatcaaagtcctcttacaatttattttccgcagcatgtcagctaagtagacccaatggggagagaaaatgcctgctttctttccctctttttctgcactgccatattcacccccaaccatccaatctgtggataattggatgttagcggtactcttccacttccgggcctggagcttggcttgtatccaagtgtatggttgctttgcctaagaggaatccctctatttcacctgttctggaggcaccagaccttgaaaagaacatgctcaaaataaaatgttatctgttatttttgtaaactcaaagttaagatgatcaaagttctaaaattccaagaatgtgcttttagacggtctcaatctaaaagcacttcaaggggtcaaagggcaaccagcttgggtgctacctcagtgttgtagtttctgatactttatgtctttgctcaccctcatccccaaactacttgaaaagggcatttggcaccactctctgaaacaacacagtcactctagcaaggcccccaaagggccctggttttacattacatttcaaactttatttgctttggggttttgtttctgttgttgttcaaatgcaaaaaaaagaaaaaaaaagaaaaaaaaaggtgactcacattgttacacatgctttaaaatatgtattcaaatgttattaaccacaatgacgacctgctttgatttaaccaagaagacggctgcggagcctagcagactcaggcctgtgggaatgggatttgttacaaatctaggtttgttactggcttcagaaagctaattaagtgctctgaaaaagacaccgtttcttgaaacaaagatggttgtattcctcactttgatgttgttttgcaagatgtttgtggaaatgttcatttgtatctggatctctgttatgtgccatttttcttctagcatcgagatacaataaaaaaaaaaaaaaagaaaagaagaagaaatactatttcaaggaaaactgctctttttgagaaacgtggacctaaactacaaagtgggaactgaggagggaactcaggagaaaggaactaactgcggagctttaatcttggccccagtgttcagccactcggaggggcgggggctgtggcccattcaggggctgctggtgggctgtagtggggtgggatgacctggccagagccaacgaggatactggagcccaaagtcaagtttagagaccagctgggaacgtgaatggggctcttgattttcttatcaaaatcaccactcctcccagcttggactaaatattctttctagcaagcagctttgtgagctccctgaagcccaaggaaacccttcggtgggagaaatttcatttctgtctgagaggattaaggcagcaggtgactccccctcctcgcctgccgtgtcctgctattctcaggcagctctaaggagaattcttatcacagttcaagtgatttccagaagttccagggcttctgagagaccatcaagggaactttaacaacttgacaaatgtccttgaagtaagatgcctcatctttagggaaaaatggggtttggatttctgcttaggcaaagtctcctgcagttcatccttctctgtcctcttcttgcttcaggcttggggaccgtccctgctgtccccactgtggtggcaatcaggacctaaggtgaagcaaacttgaagttctatctgacaagtttaggcagtaagagaaggagggaaatcggagcaaagctccctcactttattgttgagaaactggcatctggaaagaggaaggaatttgcccaaagtcagtcagctgggataaaaacctgggtgtcctgtccagaaagtgcagggtgctttctgctctgtagcaaggcagcagacatctctgagccaggcccaccaacaggcccttatctggtggttggatcatgatcccattttgcttggacatgctctcaggaagataaaaaccatggagaaacactaggccattgacaaatgatctgagacaactttagaaaacaatgtaggatgaatggaaagagaaagaaaggaaagaaagaagaaaaagaaagaaggaaagaaagaaagagaaaggaaggaaggaaagaaggaaggaaaagaaggaaggaaggaaggaatatagtgttataaatactgcactcaacattttccaaattcttgccattatttttcaaaagtttaatagtttgcagaaatagatactcaagccaaagtctgttttagagaaactttccatggaaagtcagaatttctaccacttccttttctatccacatttccagtgcagaagaaactgagaaacagagctttttgaagagaggacagggccatagcaacaaggaccttcttgggggattaatgggaggtcagtagaattaataaccctccttggatgagtgctactgttttcacatggcttcagatgctatcaacctcaaagaaatgatctcaacagagaagcttattctctcccaacttctacggtaaaatccaggagtattttctctggggatctgcccacaggacaaagtccataaaagcaagtcctgtctggaccatgtggttatctgaagcattagccatcaccagcacaacaaacggggcagggctttccaaggtggggctggtcagaagggaatctttgataagaggcccacaggcagggaaagcgaaatagggttgatgagaccaggggagacctaaaaaaaaggcagctttgtgtcttctagctccaaatatacctgccttttagctcacacactgtcctggagttctcagacctttaggggccctaacacagttcagttcatacaggggttcaaaagggacagtggcccatttgggagacctttaggatcaatgggaatcaattccattgttttgcctcagagtaaagtttctggctcggggacaattataagttgcaaaaaggatagaggcatatcccaagtcttccttcattccacaaataattacaaacaacctactgtgtgccaggcactattcttagcactggaaatacactagtgaagaagcagatgaggaccctgtttattgtttctctccaagaaattctccaagaatattgtttcttggagagaaataataaataaacaagacaatttctgaaagcaataagtgcaatcaagataattaaaggatgctaaagtgtgacttgtggggattgggagagagatgcacagacaatattaaagaggaggcattcgagctttgttgtgaacaccggaagtaacatgccgagcgcctgggggatggaaactcctatagcaccccacaggctaacagcaagcaggacaagacaaaaagggcaggtgggacatggtagagatggaccctacccaggaaacagctccatcagcatcttagcctgccccactctagccacacatacccacgtgtgctcctgagttcagtgtgcccacctcactcccacaccctcacatagacttggcaagagtaaggagggaactccatagagacattttacctatctcaggggagcagccacaaagaagcaagtcttgtaaaaggtcttttgcaaaggagagtgaacccagcaatgagagatccttaacagctagtgcccattagggggctaaacctaaagcctgggtggtgatggctcaaacgctaatgagtcagtgaatccttaccgaccccctggcctttataatctgaggcaactttggctgcagcccgggaatgtgcagggcactagggaatacaaggccttcttccctggttgtcttgtaataaaacagccatggggttgtccctccagtccgagagactgtgatgaggcctacatagcagcgatgtggtcaggtaaaaatcaggaacccactgaaatcttgggcaagccaccctgcctgcttgtgcctcggttctctcatatgtcatatataggaggtgaggactccagctccacctgccccaggtgggtgtggtgatgatgaggaaagacaagaggcttgcaaggaccctgaagaggtcggagcatcatacagattcctttattagcccacattctgatgttccctggtgagacttgccccaagcaattgctagtaaatgggggttaatttcttctccacctccctactgaacaaaaaaagaaatgccagacttactaggagaatcgagttgctttgagtttcttttgttttgttttgttttgttttgttttaaggctccccttacacaccctcctttaagctttgggttttctctcttatagtttgttgacacatgctaaaaatgtctttggagagaacttctgcctgataaacacccaattctagactgtgggtggattttcgagctgacggtggtcaattcctttcattaagcagtgatctgatttctccacatggccattctgccttcttgggggcagagtagatgggcagcagttcaccttttcagagaaagaggtcttctagccacctgggctgctactgaatggttttctccaggacgctctacctaatgattatttctataacattaagcatggtaataagtagcttccaattcaattcatcctaaagccaaagaaaatacagcaacacacacacacacacacacacacacacacacacacacacacacacaccactttatggcaattcttaactgacattcaatgacttacttcttttcttagaaaatttccaccacatttctatccccaagccaacatacaatgtgaaatgaaagccagtgcgtggagtgcagctgctaaaaattttcagcacagggctctttctgactctgctcatgagatggtatcagccacccaatgactggcgtatcttggtcctgtgtctttcttcttacgctgtgttaatgtgtttactttccatttggcagagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 46 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 47 - DNA, 8815 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 48 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 49 - DNA, 11996 bp, Homo sapiens
gcctagtctccatataaaagcggcgccgcctccccgccctctctcactccccgctcctctccgccgcgcactctccgcggcgctgggagagggcggagggggaggcggcgcgcggcgccagaggaggggggacgcagggggcggagcggagacagtaccttcggagataatcctttctcctgccgcagtggagaggagcggccggagcgagacacttcgccgaggcacagcagccggcaggatggcgaccgtggtggtggaagccaccgagccggagccgtccggcagcatcgccaacccggcggcgtccacctcgcctagcctgtcgcaccgcttccttgacagcaagttctacttgctggtggtcgtcggcgagatcgtgaccgaggagcacctgcggcgtgccatcggcaacatcgagctcggaatccgatcatgggacacaaacctgattgaatgcaacttggaccaagaactcaaactttttgtatctcgacactctgcaagattctctcctgaagtcccaggacaaaagatccttcatcaccgaagtgacgttttagaaacagtggtcctgatcaacccttctgatgaagcagtcagcaccgaggtgcgcttaatgatcactgatgctgcccgacacaagctgctcgtgctgaccgggcagtgctttgaaaataccggagagctcattctccagtccggctctttctccttccagaacttcatagagattttcaccgatcaagagatcggggagttactaagcaccacccatcctgccaacaaagccagcttaaccctgttctgtcctgaagaaggggactggaagaactccaatcttgacagacacaatctccaagacttcatcaatattaaactcaattcagcttctatcttgccagaaatggaaggactttctgagtttaccgagtatctctcagaatcagtggaagtcccatctccctttgacatcttggaacctcccacatcgggtggatttctgaagctctccaagccctgctgttatatttttccaggagggaggggcgattctgccttgtttgcagtgaatggtttcaatatgctcatcaatggcggatcagagagaaaatcctgcttctggaagctcatccgacacttagaccgagtggactccatcctgctcacccacattggggatgacaatttgcctggaataaacagcatgttacagcggaaaattgcagagctcgaggaagaacagtcccagggctccaccacaaatagtgactggatgaaaaacctcatctcccctgacttaggagttgtatttctcaatgtacctgaaaatctcaaaaatccagagccaaacatcaagatgaagagaagcatagaagaagcctgcttcactctccagtacctaaacaaattgtccatgaaaccagaacctctgtttagaagtgtaggcaatactattgatcctgtcattcttttccaaaaaatgggagtaggtaaacttgagatgtatgtgcttaatccagtcaagagcagcaaggaaatgcagtattttatgcagcagtggactggtaccaacaaagacaaggctgaattcattctgcctaatggtcaagaagtagatctcccgatttcctacttaacttcagtctcatctttgattgtgtggcatccagcaaaccctgcggagaaaatcatccgagtcctgtttcctgggaacagcacccagtacaacatcctggaagggttggaaaagctcaaacatctagactttctgaagcagccactggccacccaaaaggatctcactggccaggtgcccactcctgtggtgaaacaaacaaaactgaaacagagggctgatagccgagaaagtctgaagccagccgcaaaaccacttcctagcaaatccgtgcgcaaggagtcaaaagaagaaacccctgaggtcacaaaagtgaatcacgtggaaaagccacccaaagttgaaagcaaagaaaaggtaatggtgaaaaaagacaagccaataaaaacagagaccaaaccttcagtgactgaaaaggaggttcccagcaaagaagagccatctccagtgaaagccgaggtggctgagaagcaagccacagatgtcaaacccaaagctgccaaggagaagacggtgaaaaaggaaacaaaggtaaagcctgaagacaagaaagaggagaaagaaaagccaaagaaagaagtggctaaaaaggaggacaaaacacctatcaagaaggaggaaaaaccaaaaaaggaagaggtgaaaaaagaagtcaaaaaagagatcaagaaagaagagaaaaaagaacccaagaaagaggttaagaaagaaacaccgccaaaggaagtcaagaaggaagttaagaaggaagagaagaaggaagtgaaaaaggaagaaaaggaacccaaaaaagaaattaagaagctccctaaagacgcaaagaaatcatctactcctctgtctgaagcaaaaaaaccagctgctttaaaaccaaaagtacccaagaaggaagagtctgtcaagaaagattctgttgctgccggaaagccaaaggagaaggggaaaataaaagtcattaagaaggaaggcaaggccgcagaggctgtcgctgcagctgtcggcactggagccaccacagcagctgtcatggcggcagctggaatagcagccattggccctgccaaagaactcgaagctgagaggtcccttatgtcatctcctgaggatctaaccaaggactttgaagagttaaaggctgaagaggtcgatgtaacaaaggacatcaagcctcagctggagctaatcgaagacgaagagaaactgaaggaaactgagccagtcgaagcctacgtcatccagaaggagagagaagtcaccaaaggtcctgccgagtcccctgatgagggaatcactaccactgaaggggagggcgaatgtgaacagacacctgaggagctggagcccgtcgagaagcagggagtagacgacattgaaaaatttgaagatgaaggagccggttttgaagaatcttcagagactggagactatgaagagaaggcagaaactgaggaggctgaggagccagaagaggatggggaggaacacgtatgtgtgagcgcctccaagcacagccccactgaggatgaggaaagtgccaaggcggaggctgatgcatacatcagggagaagagggagtctgtggccagtggggatgaccgagccgaagaagacatggatgaggccattgagaaaggagaggctgaacaatctgaagaggaggctgatgaggaggacaaagctgaagatgccagagaggaggaatatgagccggaaaaaatggaagctgaagactatgtgatggctgtggtcgacaaggctgcagaggctggtggtgccgaggagcagtatggattcctcaccacaccaaccaagcaactaggagcccagtctcctggccgagaacctgcatcttcaattcatgatgagactttacctggaggctcagagagcgaggccaccgcttctgatgaggagaatcgagaagaccagcctgaggaattcactgccacctctggctacactcagtctactattgagatatccagtgagcccacccccatggatgagatgtctacccctcgagacgtgatgagtgatgagaccaacaatgaagagacggagtccccttctcaggaattcgtaaatatcaccaaatatgaatcttcattgtattctcaggaatactctaaacctgctgatgttacaccgctcaacggattttctgaaggatcaaaaacagatgccactgatggcaaggattacaatgcttcagcctctaccatatcaccaccctcttccatggaggaagacaaattcagcagatctgctttacgtgatgcttactgctctgaagtgaaagccagcaccactttggacatcaaagatagcatctcagctgtttcaagtgaaaaggtcagcccatcgaagagcccgtccctgagtccatctccaccatcacccttagaaaagacccccctgggtgaacgtagtgtgaacttctctctgacgcccaatgagattaaagtctctgcagaggcagaagtagccccggtgtctcctgaggtgacccaagaagtagttgaagaacattgtgctagtcctgaggacaagactctggaagtggtgtcaccatctcagtccgtgactggcagtgctggtcacacaccttactatcaatctcctactgacgagaaatccagtcatctccctacagaagtcattgaaaaaccaccagcagttccagtgagttttgaattcagtgatgccaaagatgagaatgaaagggcttcagtaagccccatggatgagcccgtgcctgactcagagtctcctattgaaaaagttttgtctcctttacgcagcccgcccctcattggatccgagtctgcttatgaaagttttctaagtgctgatgacaaggcttctggcagaggtgccgaaagtccttttgaagaaaagagtggaaaacaaggctctccagaccaagtaagtccagtttctgaaatgacttctactagtctttaccaagacaaacaggaagggaaaagcacagactttgcaccaataaaagaagactttggccaagaaaagaaaactgatgatgttgaagccatgagttctcaaccagcactggctctggatgaaaggaaattaggagatgtttctcccacacaaatagatgtcagtcagtttggatcttttaaagaagacactaagatgtccatttctgaaggtactgtctcagacaagtcagctactcctgttgatgagggcgtagcagaagacacgtactctcatatggagggtgtggcctcagtgtccacagcctcagtggctacgagctcatttccagagccaacaacagatgatgtgtctccatctctgcatgctgaggttggctccccacattccacagaagtagatgactccctttcagtgtctgttgtgcaaacacctaccacattccaggaaacagaaatgtctccatctaaagaagaatgcccaagaccgatgtcaatttctccaccagatttctcccctaaaactgcaaagtccaggacacccgttcaagatcacagatctgaacagtcctcaatgtctattgaatttggccaagaatctcctgagcaatcccttgctatggacttcagtcgacagtctccagatcaccctacagtgggtgcaggcgtgcttcacatcactgaaaatgggccaactgaagtggactacagtccttctgacatgcaggactccagtttatcacataagataccacctatggaggagccgtcctacacccaagataatgatctttctgagctcatctcagtatctcaggtagaggcctccccgtccacctcttctgctcataccccttctcagatcgcttctcctctccaagaagatactctatccgatgttgctcctcccagagatatgtccttatatgcctcactcacctctgaaaaagtgcaaagtctggaaggagagaagctctctccaaaatctgatatctctccactcaccccacgagagtcctctcctttatattcacctactttttcagattctacctctgcagtcaaagagaaaacagcaacttgccacagttcctcttctccaccaatagatgcagcatccgcagagccctatggcttccgtgcctcagtgttattcgatacaatgcaacaccatctagccttgaatagagatttgtccacacctggcctggagaaggacagtggagggaagacacctggtgactttagctatgcctatcaaaagcctgaggaaacaaccaggtccccagatgaagaagattatgactatgagtcttatgagaagaccacccggacctcagatgtgggtggctattactatgagaagatagagagaaccacaaaatctccaagtgacagtggctactcctatgagaccattgggaaaactaccaagacccctgaagatggtgactattcctatgaaattattgagaagaccacacggacccctgaagagggtgggtactcatatgacataagtgaaaagaccaccagcccccccgaagtgagtggttacagctatgaaaagactgagaggtctagaaggcttctggatgacatcagcaatggctatgatgactctgaggatggtggccacacacttggggaccccagctactcttatgaaaccactgagaaaattaccagtttccctgagtctgaaggttattcctatgagacatctacaaagacaacacgaacccctgatacttccacatactgttacgagactgcagagaaaatcactagaacccctcaggcatccacatattcctacgagacttcagacctatgctacactgcagaaaagaagtccccctcagaagcccgtcaggatgtcgatttatgcctcgtgtcctcttgtgaatacaagcaccccaagacagagctttcaccctctttcattaatcccaatcctcttgagtggtttgccagtgaagaacccactgaagaatctgaaaagcccctcactcaatcagggggagccccaccgcctccaggaggaaagcaacagggccgacagtgtgatgaaacccctcccacctcagtcagcgagtcagccccatcccagaccgactctgatgttcccccggagactgaagagtgcccctccatcacggccgatgccaatatcgactctgaagacgagtcggaaaccatccccacagacaaaactgtcacgtacaaacacatggacccacctccagctcccgtgcaagaccgcagcccttcgccacgccaccctgatgtgtccatggtggacccagaggccttggccattgagcagaacctgggcaaagctctaaagaaagatctgaaagagaagaccaaaaccaaaaagccaggtacaaagaccaagtcatcttcacctgtcaaaaagagtgatgggaagtctaagcccttggcagcttcaccaaaaccagcgggcttgaaagaatcctcggataaagtgtccagggtggcttctcctaagaagaaagaatctgtggaaaaggcagcaaaacccaccaccactcctgaggtcaaagctgcacgtggggaagagaaagacaaggagaccaagaatgctgccaatgcctctgcatccaagtcggccaagaccgccactgcaggaccaggaactaccaagacgaccaagtcatctgctgtgcccccaggcctccctgtgtatttggacctgtgctacattcctaaccacagcaatagtaagaatgttgatgtggaatttttcaagagagtgcggtcttcctactacgtggtgagtgggaatgaccctgctgctgaggagcccagccgggctgtcctggacgctttgttggaaggaaaggctcagtggggcagcaacatgcaggtgacactgatcccaactcatgactcagaagtgatgagggaatggtaccaggagacccatgagaaacagcaagatctcaacatcatggttttagcaagcagcagcacagtggttatgcaagatgaatccttccctgcatgcaagattgaactgtaaaaaccaaggccagccacaccacaggatctgaactttgtttccagaaattcttcaatttgaaatcaccttttctaaaaagtcaattcatctagttaagtcgctgaacaattacctgccaaatgctatactgtgtcatggtgatgcaagtcactaaatttctcagtttttgctgattgctaagggaaataacagtatttccacaatagggttcaaattcctgcaaaattacctaccccagttcatctctgctgaacatttggaaaccatgcactagccaacccaactgacttctgctaggtagaggcatttgtcttagagagagagagagcgcgggagagagtgagagagagtgagagcacaaagataacgcaggagagagagagagaaagaatgagaaagaaaaggaatgcaagagaaggagatgtaatgacagagagttctggtgagatacccagagagaaaaagagagagcagggtggggtaaggaggagaaaataaaccaacaattaggtctgcattttctcaggcagtaggcattctttagtctacataggcaaagttttccatttttgtcagtctgagtcatcaaaaagagtcttaattttctaaaacaagttggctagaagaaagtaaaaagaacaacacttgttatgagggcatgtgatattttcacatcttaattaagctccttcagtttgaaggctgcacactgacataatgtagtgagtgtagactggccatgcaagtggtttgggccccattcagaactctcagactctaaacacacaagtagattgatctaaggcatgctcccagcatttgtccacccacttagtccactctgagtcgattaacctgcatgcagcaacacccaagtccaccccaattaactgaagcaaataccaaagcagttgggagtacatatggtagacaatttgccttaggaagtgacttgaatgtacaaagatacttgatgcacttattttttaatgtgagacagcaagtttataaaacatccatataggattatagatacttaaaggaacacgtgggtgagcgtgtgtgggggtactagaagctgatctgattggtccaacagtttgatgctgagtcatgcgtgttgaatcccacttcagtgcacctgtggcctctcagtcaaacaagttgtgcctttcacagcttctttactactgcaagttcaagactgaaatggcttctatgatcagaactgggaaaacagtgaatcttatggtggaagaggttctcagcaagtgtacagtatttaccttcctttgtcttacattggctttttaaattttccattaatttcaacataattatgggaacaagtgtacagaagaattttttttttaagatatgtgagaacttttcatagatgaactttttaacaaatgttttcatttacaggaaattgcaaagaaaattctcaagtgatagtctttttttttaagtgtttcgtaagacaaaaattgaataatgttttttgaagttctggcaagattgaagtctgatattgcagtaatgatatttattaaaaacccataactaccaggaataatgatacctcccaccccttgattcccataacataaaagtgctacttgagagtgggggagaatggcatggtaggctacttttcagggccttgacaagtacatcacccagtggtatcctacatacttctttcaagatcttcaaccatgaggtaaaagagccaagttcaaagaaccctagcacaaatttgctttgggattttcttttctggaaaaaaaaaataaaagaaatagtacattgaaaacaaatgaattctcaactcctacggttcatgtagagtttagagaaaatttccatcattgtcatcattgaactgtgaacctgggaagccagatcatgattaacactgacatcaagtttcaagttgcagatcaatgcacccagtgttcagatgaggcaaacttctccgtgacaactgtgctgtgctctgtcacattacatttcctgcagactctaagatctacggagtagagaacaatgacctcattttattttctatgttagttatttatttcaaaattaacattttagttgatttttgtctgataagtctatgttttgcactgctaactatgatgagggtttaaaaaaatgcttcttcagggtcctttcactgaggacctatgcagtctacttaatgctgtgaattacatttttcaaatgtttaattttttaaagaaaattaatattctatttttgttaggcttctctagaaatgcagcttttatttattaccccatttctttcaagtccttggaaaataacatattaagggtacaagaaattaacacatgatggaaaagtcattgtgacgccaatgaatttcattgagtataaactcatctacttcaaatttattttataacacaacctaagatactcaagataattatttaatggttagctcttaagttgaattggtctacataatgcgtgggaagaaaaccagatttttagccttcttgccaaatccagacctctggttgatttttctttgacagaagatgcaagttattttccaatttcacaattaaatgtatttaacctgaacattattttgctttaaaaactataaacattgtaggagaattatagccagtcttcagttataaccactccaccctcctcactttctctctctctctctctttttttttttttttttttttgctatgggatttaatgggaaaaatatgtaaaaactgtcactagtcagctggctctttttcctatgaaatctatcagtacctttctccatccgttgttctcaatatgaccacagagcctgagtataccaagaaaaccaatattcgcattacaggttgctcctgtccttccagacacctttcctgcctgtgtgactaacctaattttgctagttccataaatacacgattagtttagtaacagccatcacaatgtaccatgtacattcatggtgagagctaaagatcgacacagacttctaggagctttgttcatactgattacttaatccaattgtatagattgaatatttgagtggaaggaatttacactctgtttaaatgatgggattctatcgagatagcactcatgatcatgacctttttggtagtattcttaaacaaaattctacagagactaaatgttagcgatgatcctccattttcaattttaaccaattctgtcccctttctcaaaaccctgagccctgtgcatgctttctcagtcttgtggtgggactggatacaatgactaacttcccctcctcccctctttaaacaccattttccatggagttcaaaaaaattttttttcttaacgttacatatcatagtgaatggtttccccagtgtatatgaatgttttaagtgtctccaatagcttatgcagtctaggagctttccaatactcatttaattaagatttaatcatttgctaatggaaatcttaccacctttcattttccctctgttaccaaatttcagctcttaggagctgctctacaattctgaatttgcttttcttgcctctctttagtcacctgtcacaggaggttcctgctcagtaatgatattgtgagttaggataataactttttttttttgtgcttcagatttagaagaaaagatcctgtttccatttgaaaggaactgtaagcttttatcttttaaccaactgaacaatacaccaaaagcagcctagggatgagcatttctttgaaagcaattaggttattcacctggtattaaaactatttactgttaaaaaatctgtgacttcatgaagttgatttttaaaggcagcatcaaaaactgaaaaggaagggaaaaaataggcagcttctctgcacttgtttggagctccccaaaacaggagccatggagaagtggcatcaagaccgggctgccctttcgagaacaccctgtggcagttcagagacacgcttttcctacactgcatgcagcccctctttccagcactggaaagaagtggtcttgagcccagctgagaagcacttcacactcctctctcttgttctgaatggtgtttgtgtcagtctgcagctgtgtatggtattatgtcttataatcctgcatcacttctatcctatccagtcatatctaatgtagaaaattagtttccagtgaaagtaatatgtagtgcttttatgatatttgtgtgcaatatcccctcttccattgaggatatttgatgtaaaggaaaaaaaaaaactcagttccacaataaaatacaaaagtggcaaaagttcaaaaaaaaaaaaaaaaaa

SEQ ID NO: 50 - protein, 2468 aa, Homo sapiens
MATVVVEATEPEPSGSIANPAASTSPSLSHRFLDSKFYLLVVVGEIVTEEHLRRAIGNIELGIRSWDTNLIECNLDQELKLFVSRHSARFSPEVPGQKILHHRSDVLETVVLINPSDEAVSTEVRLMITDAARHKLLVLTGQCFENTGELILQSGSFSFQNFIEIFTDQEIGELLSTTHPANKASLTLFCPEEGDWKNSNLDRHNLQDFINIKLNSASILPEMEGLSEFTEYLSESVEVPSPFDILEPPTSGGFLKLSKPCCYIFPGGRGDSALFAVNGFNMLINGGSERKSCFWKLIRHLDRVDSILLTHIGDDNLPGINSMLQRKIAELEEEQSQGSTTNSDWMKNLISPDLGVVFLNVPENLKNPEPNIKMKRSIEEACFTLQYLNKLSMKPEPLFRSVGNTIDPVILFQKMGVGKLEMYVLNPVKSSKEMQYFMQQWTGTNKDKAEFILPNGQEVDLPISYLTSVSSLIVWHPANPAEKIIRVLFPGNSTQYNILEGLEKLKHLDFLKQPLATQKDLTGQVPTPVVKQTKLKQRADSRESLKPAAKPLPSKSVRKESKEETPEVTKVNHVEKPPKVESKEKVMVKKDKPIKTETKPSVTEKEVPSKEEPSPVKAEVAEKQATDVKPKAAKEKTVKKETKVKPEDKKEEKEKPKKEVAKKEDKTPIKKEEKPKKEEVKKEVKKEIKKEEKKEPKKEVKKETPPKEVKKEVKKEEKKEVKKEEKEPKKEIKKLPKDAKKSSTPLSEAKKPAALKPKVPKKEESVKKDSVAAGKPKEKGKIKVIKKEGKAAEAVAAAVGTGATTAAVMAAAGIAAIGPAKELEAERSLMSSPEDLTKDFEELKAEEVDVTKDIKPQLELIEDEEKLKETEPVEAYVIQKEREVTKGPAESPDEGITTTEGEGECEQTPEELEPVEKQGVDDIEKFEDEGAGFEESSETGDYEEKAETEEAEEPEEDGEEHVCVSASKHSPTEDEESAKAEADAYIREKRESVASGDDRAEEDMDEAIEKGEAEQSEEEADEEDKAEDAREEEYEPEKMEAEDYVMAVVDKAAEAGGAEEQYGFLTTPTKQLGAQSPGREPASSIHDETLPGGSESEATASDEENREDQPEEFTATSGYTQSTIEISSEPTPMDEMSTPRDVMSDETNNEETESPSQEFVNITKYESSLYSQEYSKPADVTPLNGFSEGSKTDATDGKDYNASASTISPPSSMEEDKFSRSALRDAYCSEVKASTTLDIKDSISAVSSEKVSPSKSPSLSPSPPSPLEKTPLGERSVNFSLTPNEIKVSAEAEVAPVSPEVTQEVVEEHCASPEDKTLEVVSPSQSVTGSAGHTPYYQSPTDEKSSHLPTEVIEKPPAVPVSFEFSDAKDENERASVSPMDEPVPDSESPIEKVLSPLRSPPLIGSESAYESFLSADDKASGRGAESPFEEKSGKQGSPDQVSPVSEMTSTSLYQDKQEGKSTDFAPIKEDFGQEKKTDDVEAMSSQPALALDERKLGDVSPTQIDVSQFGSFKEDTKMSISEGTVSDKSATPVDEGVAEDTYSHMEGVASVSTASVATSSFPEPTTDDVSPSLHAEVGSPHSTEVDDSLSVSVVQTPTTFQETEMSPSKEECPRPMSISPPDFSPKTAKSRTPVQDHRSEQSSMSIEFGQESPEQSLAMDFSRQSPDHPTVGAGVLHITENGPTEVDYSPSDMQDSSLSHKIPPMEEPSYTQDNDLSELISVSQVEASPSTSSAHTPSQIASPLQEDTLSDVAPPRDMSLYASLTSEKVQSLEGEKLSPKSDISPLTPRESSPLYSPTFSDSTSAVKEKTATCHSSSSPPIDAASAEPYGFRASVLFDTMQHHLALNRDLSTPGLEKDSGGKTPGDFSYAYQKPEETTRSPDEEDYDYESYEKTTRTSDVGGYYYEKIERTTKSPSDSGYSYETIGKTTKTPEDGDYSYEIIEKTTRTPEEGGYSYDISEKTTSPPEVSGYSYEKTERSRRLLDDISNGYDDSEDGGHTLGDPSYSYETTEKITSFPESEGYSYETSTKTTRTPDTSTYCYETAEKITRTPQASTYSYETSDLCYTAEKKSPSEARQDVDLCLVSSCEYKHPKTELSPSFINPNPLEWFASEEPTEESEKPLTQSGGAPPPPGGKQQGRQCDETPPTSVSESAPSQTDSDVPPETEECPSITADANIDSEDESETIPTDKTVTYKHMDPPPAPVQDRSPSPRHPDVSMVDPEALAIEQNLGKALKKDLKEKTKTKKPGTKTKSSSPVKKSDGKSKPLAASPKPAGLKESSDKVSRVASPKKKESVEKAAKPTTTPEVKAARGEEKDKETKNAANASASKSAKTATAGPGTTKTTKSSAVPPGLPVYLDLCYIPNHSNSKNVDVEFFKRVRSSYYVVSGNDPAAEEPSRAVLDALLEGKAQWGSNMQVTLIPTHDSEVMREWYQETHEKQQDLNIMVLASSSTVVMQDESFPACKIEL

SEQ ID NO: 51 - DNA, 3283 bp, Homo sapiens
aggagaagagcaaagaaaagcagtccgtctggatttgtttgcccaggactggcgccgcgcacgcggatcgccgaggggagtgcggtcggagtcaccgcgcccccgcctccccgcccgggcagctgaggccgggggttggagcgctgcccccgcgcacagtccccgagcgcccgacgtctccgcgcaggttcttgaagcagctgggcctggggcgcccactaatgtggccctgagggccggagcccgcaccgacgggagcgggagccggagcagctgcgggcgccgagtggccggtgcgcccggcggagcgcgcgtgcgtggccagcgcgctccccgcttctgcttggctttccggcttaattttcctcggcgggattaaagttggaaattgaccggagaattgagttgccggggaacagagccccggccgccgccagagcgatgttcccgcagagccggcacccgacgccgcaccaggctgcaggccagcccttcaagttcactatcccggagtccctggaccggattaaagaggaattccagttcctgcaggcgcagtatcacagccttaaattggaatgtgagaaactggcaagtgaaaagacagaaatgcagaggcactatgtgatgtattatgaaatgtcatatggattaaacattgaaatgcacaaacagactgaaatcgccaagagattgaatacgatttgtgcacaagtcatcccatttctgtctcaggaacatcaacaacaggtggcccaggctgttgaacgtgccaaacaggtgaccatggcagagttgaatgccatcatcgggcagcagcagttgcaagctcagcatctttctcatggccacggacccccagttccccttacgcctcacccttcgggacttcagcctcctggaatcccgcccctcgggggcagtgccggccttcttgcgctgtctagtgctctgagtgggcagtctcacttggcaataaaagatgacaagaagcaccacgatgcagagcaccacagagacagagagccgggcacaagtaattccctcctggtcccagacagtctaagaggcacagataaacgcagaaatggacctgaattttccaatgacatcaagaaaaggaaggtggatgataaggactccagccactatgacagtgatggtgacaaaagcgatgacaacttagttgtggatgtgtctaatgaggacccttcttctccgcgagcaagccctgcccactcgccccgggaaaatggaatcgacaaaaatcgcctgctaaagaaggatgcttctagcagtccagcttccacggcctcctcggcaagttccacttctttgaaatccaaagaaatgagcttgcatgaaaaagccagcacgcctgttctgaaatccagcacaccaacgcctcggagcgacatgccaacgccgggcaccagcgccactccaggcctccgtccaggtctcggcaagcctccagccatagaccccctcgttaaccaagcggcagctggcttgaggacacccctggcagtgcccggcccatatcctgctccttttgggatggtcccccacgctggcatgaacggcgagctgaccagcccaggcgctgcctacgccagtttacacaacatgtcgccccagatgagcgccgcagccgccgcggccgccgtggtggcctacgggcgctcccccatggtggggtttgatcctccccctcacatgagagtacctaccattcctccaaacctggcaggaatccctggggggaaacctgcatactccttccacgttactgcagacggtcagatgcagcctgtcccttttccccccgacgccctcatcggacccggaatcccccggcatgctcgccagatcaacaccctcaaccacggggaggtggtgtgcgctgtgaccatcagcaaccccacgagacacgtgtacacaggcgggaagggctgcgtcaaggtctgggacatcagccaccctggcaataagagccctgtctcccagctcgactgtctgaacagagacaattatatccgttcctgtaaattgctacccgatggctgcactctcatagtgggaggggaagccagtactttgtccatttgggacctggcggctccaaccccgcgcatcaaggcggagctgacgtcctcggcccccgcctgctacgccctggccatcagccccgattccaaggtctgcttctcatgctgcagcgacggcaacatcgctgtgtgggatctgcacaaccagacactagtgaggcaattccagggccacacagacggagccagctgtattgacatttctaatgatggcaccaagctctggacgggtggtttggacaacacagtcaggtcctgggacctgcgcgaggggcggcagctgcagcagcacgacttcacctcccagatcttctccctggggtactgccccaccggggagtggctggcagtgggcatggagagcagcaatgtggaggtgctgcacgtgaacaagcctgacaagtaccagctgcacctgcatgagagctgcgtgctgtccctgaaatttgcttactgtggtaaatggtttgtgagtactggaaaagataacctcctcaatgcttggcggaccccctatggagccagcatattccagtccaaagagtcctcgtcagtgcttagctgtgacatctctgtggatgataagtacatagtcactggctcgggggacaagaaggctacagtctatgaagtcatctactgaaaacattatgtggtttaacgtttatagttgaattgggccaaaatgtttcgaatttatagaaatagaaaagttgtaactttaaaagagaaaaaaaattacaaacacctgtttccaaaccttgacagaaaactactttgagtctacaaagaggaggcgacaagtccatcagcagaaagtcacctgtctacatagaccaaatggagcaccaaggccaagcggacagaggggccatgggttgtaggattgaggaacggaatctgccgactcacatgacagcccattctttctttctgggtgatctggggatcacgccttgcccaagtgtgagattacctttctgttccttgcagttcacctcactttccgtcctttgtagagcagtggtgtctccaatgaacttgtttcctggttttgcatcttgtgaaatgtttttttgtatttttgttgaaggttaaacatttgtataaattgtaaatatatttggtttattacagtaaaggctttagtaccaataaaaaaaaaaaaaaaa

SEQ ID NO: 52 - protein, 770 aa, Homo sapiens
MFPQSRHPTPHQAAGQPFKFTIPESLDRIKEEFQFLQAQYHSLKLECEKLASEKTEMQRHYVMYYEMSYGLNIEMHKQTEIAKRLNTICAQVIPFLSQEHQQQVAQAVERAKQVTMAELNAIIGQQQLQAQHLSHGHGPPVPLTPHPSGLQPPGIPPLGGSAGLLALSSALSGQSHLAIKDDKKHHDAEHHRDREPGTSNSLLVPDSLRGTDKRRNGPEFSNDIKKRKVDDKDSSHYDSDGDKSDDNLVVDVSNEDPSSPRASPAHSPRENGIDKNRLLKKDASSSPASTASSASSTSLKSKEMSLHEKASTPVLKSSTPTPRSDMPTPGTSATPGLRPGLGKPPAIDPLVNQAAAGLRTPLAVPGPYPAPFGMVPHAGMNGELTSPGAAYASLHNMSPQMSAAAAAAAVVAYGRSPMVGFDPPPHMRVPTIPPNLAGIPGGKPAYSFHVTADGQMQPVPFPPDALIGPGIPRHARQINTLNHGEVVCAVTISNPTRHVYTGGKGCVKVWDISHPGNKSPVSQLDCLNRDNYIRSCKLLPDGCTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMESSNVEVLHVNKPDKYQLHLHESCVLSLKFAYCGKWFVSTGKDNLLNAWRTPYGASIFQSKESSSVLSCDISVDDKYIVTGSGDKKATVYEVIY

SEQ ID NO: 53 - DNA, 3204 bp, Homo sapiens
aatgggcgggcagcatccacatgacccgcgccggcgggagggcgtggggaggcaggccaggaacgcacgctgcctggccgtatcgccgcccccaccgccgccgccgccgggactagaagtgagccgcccgggtcccaaacgccagccagccagtcagtgggtcccgcagtcgcccgcaaccggggcgaatcatggcggccgccaaggtggctttaaccaagagagcagatccagctgagcttagaacaatatttttgaagtatgcaagcattgagaaaaacggtgaatttttcatgtcccccaatgactttgtcactcgatacttgaacatttttggagaaagccagcctaatccaaagactgtggaacttttaagtggagtggtggatcagaccaaagatggattaatatcttttcaagaatttgttgcctttgaatctgtcctgtgtgcccctgatgctttgtttatggtagcctttcagctgtttgacaaagctggcaaaggagaagtaacttttgaggatgttaagcaagtttttggacagaccacaattcatcaacatattccatttaactgggattcagaatttgtgcaactacattttggaaaagaaagaaaaagacacctgacatatgcggaatttactcagtttttattggaaatacaactggagcacgcaaagcaagcctttgtgcaacgggacaatgctaggactgggagagtcacagccatcgacttccgagacatcatggtcaccatccgcccccatgtcttgactccttttgtagaagaatgtctagtagctgctgctggaggtaccacatcccatcaagttagtttctcctattttaatggatttaattcgctccttaacaacatggaactcattagaaagatctatagcactctggctggcaccaggaaagatgttgaagtgactaaggaggagtttgttctggcagctcagaaatttggtcaggttacacccatggaagttgacatcttgtttcagttagcagatttatatgagccaaggggacgtatgaccttagcagacattgaacggattgctcctctggaagagggaactctgccctttaacttggctgaggcccagaggcagaaggcctcaggtgattcagctcgaccagttcttctacaagttgcagagtcggcctacaggtttggtctgggttctgttgctggagctgttggagccactgctgtgtatcctatcgatcttgtaaaaactcgaatgcagaaccaacgatcaactggctcttttgtgggagaactcatgtataaaaacagctttgactgttttaagaaagtgctacgctatgaaggcttctttggactgtatagaggtctgttgccacagttattgggagttgccccagagaaggccataaaacttacagtgaacgattttgtgagggataaatttatgcacaaagatggttcggtcccacttgcagcagaaattcttgctggaggctgcgctggaggctcccaggtgattttcacaaatcctttagaaatcgtcaagatccgtttgcaagtggcaggagaaatcaccactggtcctcgagtcagtgctctgtctgtcgtgcgggacctggggttttttgggatctacaagggtgccaaagcatgctttctgcgggacattcctttctcggccatctactttccgtgctatgctcatgtgaaggcttcctttgcaaatgaagatgggcaggttagcccaggaagcctgctcttagctggtgccatagctggtatgcctgcagcatctttagtgacccctgctgatgttatcaagacgagattacaggtggctgcccgggctggccaaaccacttacagcggagtgatagactgctttagaaagatactgcgtgaagaaggaccaaaagctctgtggaagggagctggtgctcgtgtatttcgatcctcaccccagtttggtgtaactttgctgacttacgaattgctacagcgatggttctacattgattttggaggagtaaaacccatgggatcagagccagttcctaaatccaggatcaacctgcctgccccgaatcctgatcacgttgggggctacaaactggcagttgctacatttgcagggattgaaaacaaatttggactttacctacctctcttcaagccatcagtatctacctcaaaggctattggtggaggcccataggaagatcagccctgggatagtgctgtctttttgtgggtactgcagtaaagaacatccctcctgggaatgaagcaatgcttcatcccttttacgtccatctcttgtttaaattcaagtccaggcttttttatcatgtgaaatcattcattttctgggtgttttcttaaccagatcattgtgaaattattcataattattatttggccctctgcccagaaacctttgtttgcatctgaaaattgatgggatttggtcaacactaacatgatttggggaaaggagcaagtcagaatagaaattagtactcccctccttgaactaggattgtagtcccaaagaggctactgtaaggcaatcatggtgctcagagcagtgtttcgtgtgtgttttaaactggtaggaaactaggtgcatatttataaaaataaaaaacactgggagaaatgaaaaaatatatatcaaatatattcagcctggcttcaaattgtaagcatgcacaaattctgtctctggattatattatgaagcttttatgtgaaacatgtttctttgtaatgaaaaccacattggagatgtttagtaatcatattgttactggtaccaagactactagggaaatgcctttgtactttagggaagtacttttggcattttactgtacagacagaaaaaactgagatgtagcccctctcctggaagtgctaattttgaaaaactgctcatatgatgtacatgtactgattactgcctattttaataaacactcttgaaaaatgcatgttgccctgttgctgcctgccctattctcctcatctccccatcattggtacccacttgcttttaaaatccactttatcttgaataatgtaagacaaatatgttctgacataagtatttaattcatgttgccttgcataatggtcagaggcgcatgaatttgtgaaggtggaaataaactatttgtaaagtgaaaaaaaaaaaaaaa

SEQ ID NO: 54 - protein, 675 aa, Homo sapiens
MAAAKVALTKRADPAELRTIFLKYASIEKNGEFFMSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 55 - DNA, 4264 bp, Homo sapiens
agagcgctgcggccgcggcggtgcagcagaggcgcctcgggcaggaggagggcggcttctgcgagggcagcctgaggtattaaaaagtgtcagcaaactgcattgaataacagacatcctaagaggggatattttccacctctataatgaagaaaagcaggagtgtgatgacggtgatggctgatgataatgttaaagattattttgaatgtagcttgagtaaatcctacagttcttccagtaacacacttgggatcgacctctggagagggagaaggtgttgctcaggaaacttacagttaccaccactgtctcaaagacagagtgaaagggcaaggactcctgagggagatggtatttccaggccgaccacactgcctttgacaacgcttccaagcattgctattacaactgtaagccaggagtgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgtacagtccctggccctgttccctacctcctctcttcaccccgttaggctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagcttttacttttgtatagcttgataggggcagggggcaatgggatgtagtttttacccaggttctatccaaatctatgtgggcatgagttgggttataactggatcctactatcattgtggctttggttcaaaaggaaacactacatttgctcacagatgattcttctgaatgctcccgaactactgactttgaagaggtagcctcctgcctgccattaagcaggaatgtcatgttccagttcattacaaaagaaaacaataaaacaatgtgaatttttataataaaatgtgaactgatgtagcaaattacgcaaatgtgaagcctcttctgataacacttgttaggcctcttactgatgtcagtttcagtttgtaaaatatgtttcatgctttcagttcagcattgtgactcagtaattacagaaaatggcacaaatgtgcatgaccaatgtatgtctatgaacactgcattgtttcaggtggacattttatcattttcaaatgtttctcacaatgtatgttatagtattattattatatattgtgttcaaatgcattctaaagagacttttatatgaggtgaataaagaaaagcatgattagattaaaaaaa

SEQ ID NO: 56 - protein, 736 aa, Homo sapiens
MKKSRSVMTVMADDNVKDYFECSLSKSYSSSSNTLGIDLWRGRRCCSGNLQLPPLSQRQSERARTPEGDGISRPTTLPLTTLPSIAITTVSQECFDVENGPSPGRSPLDPQASSSAGLVLHATFPGHSQRRESFLYRSDSDYDLSPKAMSRNSSLPSEQHGDDLIVTPFAQVLASLRSVRNNFTILTNLHGTSNKRSPAASQPPVSRVNPQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 57 - DNA, 4214 bp, Homo sapiens
gcgcctcgggcggcttctcgccgctcccaggtctggctggctggaggagtctcagctctcagccgctcgcccgcccccgctccgggccctcccctagtcgccgctgtggggcagcgcctggcgggcggcccgcgggcgggtcgcctcccctcctgtagcccacacccttcttaaagcggcggcgggaagatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagcgttttggtacacggataagagacctgaaattagccagggacctctgctgtgtgtctctgccaatctgctgggctggtccctctcatttttaccagtctgagtgacaggtccccttcgctcatcattcagatggctttccagatgaccaggacgagtgggatattttgcccccaacttggctcggcatgtgaattcttagctctgcaaggtgtttatgcctttgcgggtttcttgatgtgttcgcagtgtcaccccagagtcagaactgtacacatcccaaaatttggtggccgtggaacacattcccggtgatagaattgctaaattgtcgtgaaataggttagaatttttctttaaattatggttttcttattcgtgaaaattcggagagtgctgctaaaattggattggtgtgatctttttggtagttgtaatttaacagaaaaacacaaaatttcaaccattcttaatgttacgtcctccccccacccccttctttcagtggtatgcaaccactgcaatcactgtgcatatgtcttttcttagcaaaaggattttaaaacttgagccctggaccttttgtcctatgtgtgtggattccagggcaactctagcatcagagcaaaagccttgggtttctcgcattcagtggcctatctccagattgtctgatttctgaatgtaaagttgttgtgtttttttttaaatagtagtttgtagtattttaaagaaagaacagatcgagttctaattatgatctagcttgattttgtgttgatccaaatttgcatagctgtttaatgttaagtcatgacaatttatttttcttggcatgctatgtaaacttgaatttcctatgtatttttattgtggtgttttaaatatggggaggggtattgagcattttttagggagaaaaataaatatatgctgtagtggccacaaataggcctatgatttagctggcaggccaggttttctcaagagcaaaatcaccctctggccccttggcaggtaaggcctcccggtcagcattatcctgccagacctcggggaggatacctgggagacagaagcctctgcacctactgtgcagaactctccacttccccaaccctccccaggtgggcagggcggagggagcctcagcctccttagactgacccctcaggcccctaggctggggggttgtaaataacagcagtcaggttgtttaccagccctttgcacctccccaggcagagggagcctctgttctggtgggggccacctccctcagaggctctgctagccacactccgtggcccaccctttgttaccagttcttcctccttcctcttttcccctgcctttctcattccttccttcgtctccctttttgttcctttgcctcttgcctgtcccctaaaacttgactgtggcactcagggtcaaacagactatccattccccagcatgaatgtgccttttaattagtgatctagaaagaagttcagccgaacccacaccccaactccctcccaagaacttcggtgcctaaagcctcctgttccacctcaggttttcacaggtgctcccaccccagttgaggctcccacccacagggctgtctgtcacaaacccacctctgttgggagctattgagccacctgggatgagatgacacaaggcactcctaccactgagcgcctttgccaggtccagcctgggctcaggttccaagactcagctgcctaatcccagggttgagccttgtgctcgtggcggaccccaaaccactgccctcctgggtaccagccctcagtgtggaggctgagctggtgcctggccccagtcttatctgtgcctttactgctttgcgcatctcagatgctaacttggttctttttccagaagcctttgtattggttaaaaattattttccattgcagaagcagctggactatgcaaaaagtatttctctgtcagttccccactctataccaaggatattattaaaactagaaatgactgcattgagagggagttgtgggaaataagaagaatgaaagcctctctttctgtccgcagatcctgacttttccaaagtgccttaaaagaaatcagacaaatgccctgagtggtaacttctgtgttattttactcttaaaaccaaactctaccttttcttgttgttttttttttttttttttttttttttttggttaccttctcattcatgtcaagtatgtggttcattcttagaaccaagggaaatactgctccccccatttgctgacgtagtgctctcatgggctcacctgggcccaaggcacagccagggcacagttaggcctggatgtttgcctggtccgtgagatgccgcgggtcctgtttccttactggggatttcagggctgggggttcagggagcatttccttttcctgggagttatgaccgcgaagttgtcatgtgccgtgcccttttctgtttctgtgtatcctattgctggtgactctgtgtgaactggcctttgggaaagatcagagagggcagaggtggcacaggacagtaaaggagatgctgtgctggccttcagcctggacagggtctctgctgactgccaggggcgggggctctgcatagccaggatgacggctttcatgtcccagagacctgttgtgctgtgtattttgatttcctgtgtatgcaaatgtgtgtatttaccattgtgtagggggctgtgtctgatcttggtgttcaaaacagaactgtatttttgcctttaaaattaaataatataacgtgaataaatgaccctatctttgtaacaaaaaaaaaaaaaaa

SEQ ID NO: 58 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 59 - DNA, 3143 bp, Homo sapiens
cggagctggagctcccaccgccgccgcccgtgcctccggctgccggcgcccctgcctttggctcttcctccccactcgcccgctccccctggcggagccggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatggctggtctgggccccggcgtaggcgattcagaggggggaccccggcccctgttttgcagaaagggggccctgaggcagaaggtggtccacgaagtcaagagccacaagttcaccgctcgcttcttcaagcagcccaccttctgcagccactgcaccgacttcatctggggtatcggaaagcagggcctgcaatgtcaagtctgcagctttgtggttcatcgacgatgccacgaatttgtgaccttcgagtgtccaggcgctgggaagggcccccagacggacgacccccggaacaaacacaagttccgcctgcatagctacagcagccccaccttctgcgaccactgtggctccctcctctacgggcttgtgcaccagggcatgaaatgctcctgctgcgagatgaacgtgcaccggcgctgtgtgcgtagcgtgccctccctgtgcggtgtggaccacaccgagcgccgcgggcgcctgcagctggagatccgggctcccacagcagatgagatccacgtaactgttggcgaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttcaacctgaagccaggggatgtggagcgccggctcagcgtggaggtgtgggactgggaccggacctcccgcaacgacttcatgggggccatgtcctttggcgtctcggagctgctcaaggcgcccgtggatggctggtacaagttactgaaccaggaggagggcgagtattacaatgtgccggtggccgatgctgacaactgcagcctcctccagaagtttgaggcttgtaactaccccctggaattgtatgagcgggtgcggatgggcccctcttcctctcccatcccctccccttcccctagtcccaccgaccccaagcgctgcttcttcggggcgagtccaggacgcctgcacatctccgacttcagcttcctcatggttctaggaaaaggcagttttgggaaggtgatgctggccgagcgcaggggctctgatgagctctacgccatcaagatcttgaaaaaggacgtgatcgtccaggacgacgatgtggactgcacgctggtggagaaacgtgtgctggcgctggggggccggggtcctggcggccggccccacttcctcacccagctccactccaccttccagaccccggaccgcctgtatttcgtgatggagtacgtcaccgggggagacttgatgtaccacattcaacagctgggcaagtttaaggagccccatgcagcgttctacgcggcagaaatcgctatcggcctcttcttccttcacaatcagggcatcatctacagggacctgaagctggacaatgtgatgctggatgctgagggacacatcaagatcactgactttggcatgtgtaaggagaacgtcttccccgggacgacaacccgcaccttctgcgggaccccggactacatagccccggagatcattgcctaccagccctatgggaagtctgtcgattggtggtcctttggagttctgctgtatgagatgttggcaggacagcctcccttcgatggggaggacgaggaggagctgtttcaggccatcatggaacaaactgtcacctaccccaagtcgctttcccgggaagccgtggccatctgcaaggggttcctgaccaagcacccagggaagcgcctgggctcagggcctgatggggaacctaccatccgtgcacatggctttttccgctggattgactgggagcggctggaacgattggagatcccgcctcctttcagaccccgcccgtgtggccgcagcggcgagaactttgacaagttcttcacgcgggcggcgccagcgctgacccctccagaccgcctagtcctggccagcatcgaccaggccgatttccagggcttcacctacgtgaaccccgacttcgtgcacccggatgcccgcagccccaccagcccagtgcctgtgcccgtcatgtaatctcacccgccgccactaggtgtccccaacgtcccctccgccgtgccggcggcagccccacttcacccccaacttcaccaccccctgtcccattctagatcctgcaccccagcattccagctctgcccccgcgggttctagacgcccctcccaagcgttcctggccttctgaactccatacagcctctacagccgtcccgcgttcaagacttgagcggagcccgatattctccctgaccttagcgttctggactctgccccaatcgggtccagagaccacaccactaaccatccccaactccatggggttcgagactccatcttggtagttctgtgcctccccccagaccccgcccctggggaaatagcctcacggggttggctgttccagactcaggttccagaacagccctcggcctccgaggctccccgcctccactctagttctagatgagtgggaggcgtgcccccctcctccagtacgtcccgctgctgtgctctggggatttctgggatatatggaggattctttccccagaggctcccaatcagcttttgttctagacttccccatcccgaagccatcacttctccccgcagcccgcctgccgtgcatggctcctgtctggctcggacccaccccaactctccccagtgcctgccactctctgggactctcctcctcccctcctcttcccttagcctctcccacccggccacagctgctggagaataaatttgggatgctgatgaaaaaaaaaaaaaaa

SEQ ID NO: 60 - protein, 697 aa, Homo sapiens
MAGLGPGVGDSEGGPRPLFCRKGALRQKVVHEVKSHKFTARFFKQPTFCSHCTDFIWGIGKQGLQCQVCSFVVHRRCHEFVTFECPGAGKGPQTDDPRNKHKFRLHSYSSPTFCDHCGSLLYGLVHQGMKCSCCEMNVHRRCVRSVPSLCGVDHTERRGRLQLEIRAPTADEIHVTVGEARNLIPMDPNGLSDPYVKLKLIPDPRNLTKQKTRTVKATLNPVWNETFVFNLKPGDVERRLSVEVWDWDRTSRNDFMGAMSFGVSELLKAPVDGWYKLLNQEEGEYYNVPVADADNCSLLQKFEACNYPLELYERVRMGPSSSPIPSPSPSPTDPKRCFFGASPGRLHISDFSFLMVLGKGSFGKVMLAERRGSDELYAIKILKKDVIVQDDDVDCTLVEKRVLALGGRGPGGRPHFLTQLHSTFQTPDRLYFVMEYVTGGDLMYHIQQLGKFKEPHAAFYAAEIAIGLFFLHNQGIIYRDLKLDNVMLDAEGHIKITDFGMCKENVFPGTTTRTFCGTPDYIAPEIIAYQPYGKSVDWWSFGVLLYEMLAGQPPFDGEDEEELFQAIMEQTVTYPKSLSREAVAICKGFLTKHPGKRLGSGPDGEPTIRAHGFFRWIDWERLERLEIPPPFRPRPCGRSGENFDKFFTRAAPALTPPDRLVLASIDQADFQGFTYVNPDFVHPDARSPTSPVPVPVM

SEQ ID NO: 61 - DNA, 2258 bp, Homo sapiens
agggaagctcctataaagggctgcatctctggactcacagctatcagaccattgagatgtggaagctgttgctgtgggttgggctggttcttgtgctgaaacaccacgatggtgctgcccataaactcgtgtgttatttcaccaactgggcacacagtcggccaggccctgcctcgatcttgccccatgacctggacccctttctctgcacccacctgatatttgcctttgcctcaatgaacaacaatcagattgttgctaaggatctccaggatgagaaaattctctacccagagttcaacaaactaaaggagaggaacagagagctgaaaacactactgtccatcggcgggtggaactttggcacctcaagattcaccactatgttgtccacatttgccaaccgtgaaaagtttattgcttcagttatatcccttctgaggacacatgactttgatggtcttgaccttttcttcttatatcctggactaagaggcagccccatgcatgaccggtggacttttctcttcttaattgaagagctcctgtttgccttccggaaggaggcactgctcaccatgcgcccgaggctgctgctgtctgctgctgtttctggggtcccacacatcgtccaaacatcctatgatgtgcgctttctaggaagactcctggatttcatcaatgtcttgtcttatgacttacatggaagttgggaaaggttcacaggacataatagccccctcttctctctgcctgaagaccccaaatcttcggcatatgctatgaattattggagaaagcttggggcaccctcagagaagctcatcatggggatccccacctatggacgtacctttcgcctcctcaaagcctctaagaatgggttgcaggccagagcgatcggaccagcatctccagggaagtacaccaagcaagaaggcttcttggcttattttgagatttgttcctttgtctggggagcgaagaagcactggattgattaccagtatgtcccgtatgccaacaaggggaaagagtgggttggctatgacaatgccatcagcttcagttacaaggcatggtttataaggcgagagcattttgggggggccatggtgtggacattggacatggatgacgtcaggggcacgttctgtggcactggccctttcccccttgtctacgtattgaatgatatcctggtgcgggctgagttcagttcaacttctttaccacaattttggctgtcatctgctgtgaattcttcaagcactgaccctgaaaggctggctgtgaccacggcatggaccactgatagtaagattttgcccccaggaggagaggctggggtcactgagatccacggaaagtgtgaaaatatgactataacccctagaggtacaactgtgacccctacaaaggaaactgtatcccttggaaagcacactgtagctctaggagagaagactgagatcactggggcaatgaccatgacttctgtgggtcatcagtccatgacccctggagagaaggccctgacccctgtgggtcatcaatctgtgaccactggacagaagaccctgacctctgtgggttatcagtctgtgacccctggggaaaagaccctgacccctgtgggtcatcagtctgtgacccctgtgagtcatcagtctgtgagccctggaggaacgactatgacccctgtccattttcagactgagacccttagacagaatacagtggcccctagaaggaaggctgtggcccgtgaaaaggtgactgtcccctccagaaacatatcagtcacccctgaagggcagactatgcctttaagaggggagaatttgacttctgaggtgggcactcaccccaggatgggtaacttgggtcttcagatggaagctgaaaacaggatgatgctgtcctccagccccgtcatccagctcccggaacaaactcctctagcttttgacaaccgctttgttcccatctatggaaaccattcctctgtcaactcagtaacccctcaaacaagtcctctttctctaaaaaaagaaatcccagaaaactctgctgtggatgaagaagcctaagcccctctggtgtcagaaaccagggaaaacccttgtcttttcttctaagtgacatgttggaagccttctcatcccggggcaaagcaggcatcaaaaccagaataggccaatctcttttccattaaataaactgtaaacacaagaacccaaaaaaaaaaaaaaaaa

SEQ ID NO: 62 - protein, 678 aa, Homo sapiens
MWKLLLWVGLVLVLKHHDGAAHKLVCYFTNWAHSRPGPASILPHDLDPFLCTHLIFAFASMNNNQIVAKDLQDEKILYPEFNKLKERNRELKTLLSIGGWNFGTSRFTTMLSTFANREKFIASVISLLRTHDFDGLDLFFLYPGLRGSPMHDRWTFLFLIEELLFAFRKEALLTMRPRLLLSAAVSGVPHIVQTSYDVRFLGRLLDFINVLSYDLHGSWERFTGHNSPLFSLPEDPKSSAYAMNYWRKLGAPSEKLIMGIPTYGRTFRLLKASKNGLQARAIGPASPGKYTKQEGFLAYFEICSFVWGAKKHWIDYQYVPYANKGKEWVGYDNAISFSYKAWFIRREHFGGAMVWTLDMDDVRGTFCGTGPFPLVYVLNDILVRAEFSSTSLPQFWLSSAVNSSSTDPERLAVTTAWTTDSKILPPGGEAGVTEIHGKCENMTITPRGTTVTPTKETVSLGKHTVALGEKTEITGAMTMTSVGHQSMTPGEKALTPVGHQSVTTGQKTLTSVGYQSVTPGEKTLTPVGHQSVTPVSHQSVSPGGTTMTPVHFQTETLRQNTVAPRRKAVAREKVTVPSRNISVTPEGQTMPLRGENLTSEVGTHPRMGNLGLQMEAENRMMLSSSPVIQLPEQTPLAFDNRFVPIYGNHSSVNSVTPQTSPLSLKKEIPENSAVDEEA

SEQ ID NO: 63 - DNA, 6382 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagatagtgtgtatatatatatatatgggtgggtgttttgttgcagctgctgatctttttctttgcagatggtacaaactctcccgagtcaatttcctgggcctatgtccccacctagctgactgaagttatcaacaggggtccagtttgtgcaggctgctagccctattggaagagtggggatgaggtgggagaaagcaaccacaacgtgtgtgggcaacctcaattggcactcataaaatgttagaatgtcaactctctcccttggccactaaatctctcacagggtagtttttcttgcctaactcaggtttacaaatcaatgtgtatgccttgggggaccaatggcctctttcctcccaaataaaccactggctttctctttgtccccctaggttatagctgaggagcccactccaattagtttataggattcaaagcctctttttaaaaacatctctgagcttatgaggaaagacttcaagtttcccaaatctagtggaggacagggcaagggaggaaagataggtacaggagtccacaggaggccaggttttggcacccctttgtcaggaattcagcttccttactagggatgaagaaaataagtgtggggctttgtgtctatgctaccagaaggaggagaggatgacacttcctctctgtttcccagattagagaacagtgaacccaatgctgcctgttggctagaaaacaagtgttaacttgcttctgagagacccttttctctgtccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 64 - protein, 626 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 65 - DNA, 2796 bp, Homo sapiens
gcggcggcttccaacaggctctggggcgccgagcggacaggaacgcagcacgggggctccgaggcggggtctgggtgttgaggggcgactggagccatggcggagtcggcgcctgctcggcacaggagaaaacgacgctccacacctttaacttcttccacacttccttcacaagcaacagagaaaagctcctattttcagaccaccgagatttcactctggacggtggtggccgctattcaggctgtggagaagaagatggagtcccaggctgcccggctacagagcctggaggggcgcacggggacagccgagaagaagctggctgactgcgagaagatggccgtggagttcgggaaccagctggagggcaagtgggccgtgctggggaccctgctgcaggagtatgggctactgcagaggcggctggagaacgtggagaacctgctgcgcaacaggaacttctggatcctgcggctgcccccgggcagcaagggggaggcccccaaggtgtccaggtcactggagaatgatggcgtctgtttcaccgagcaggaatgggagaatctggaggattggcagaaggagctctacagaaacgtgatggagagtaactatgagacactggtctctctgaaggtccttggccagacagagggagaagcggagttgggtacagagatgctgggtgacttggaagaggaaggtcctggtggtgcccacccagcaggtggggtcatgatcaaacaggagctacagtatacacaggaaggccctgcggatcttcctggagagttctcatgcattgctgaagagcaggctttcctgagcccagagcagaccgaactctggggtggtcagggcagttctgtcctcttggaaacaggtcctggggactctactctagaggagcctgttggtagtagagttcctagcagcagcagaactgtgggctgcccgaagcagaaatctcataggcaggtacagctggaccaggaatgtgggcagggcctgaagctgaaaaaggacacttcccgcccctacgaatgttctgagtgtgagatcaccttccgctataagcagcagctggccacacatctgcgcagccactctgggtgggggtcttgtacacctgaggagccagaggagagccttaggcccaggccacggctgaaaccacagaccaaaaaggccaagctgcatcagtgtgatgtgtgcctgaggagcttcagctgcaaggtgagcctggtgacccatcagcgttgccacctgcaggaggggcccagtgccggccagcatgtccaagagaggttctcacccaacagcctggttgccctgcctggccacatcccttggaggaaaagccggagttccctcatctgtggttactgtggcaagagcttcagtcacccatctgacttggtgcggcaccagcgcatccacacgggtgagcggccctacagctgcactgagtgtgagaagagctttgtccagaagcagcacctcctgcagcaccagaagatccaccagcgggagcggggtgggctggccctggagcccggaaggcccaatggcctgctttaagggtgcagcccctcgcccgtctgggggatggaggggggtggcattggttcccccgaagagacactgcagtcagggactgagttcttcctgagggcagttgtttgtgattgccttcccttgtcccagtaccaagccaagcccaaaggctgtcctgaaaaccctgtggaagaagagtccaggccaggtcttcatcctgctgccaagtttgctgtttcttggcaccttcaggtctctggttttctcattcatgccaatgcttgtgggctggggttggcgttctgaccccacagggactggtggctggttccagggctcgtcccggcatttcatgtcttcccacggggttgagtcgggccataggggtgagcagctgcctggaagagttctgggaagtataaccctccattttttcttgttttataatctctttgtttaataataagtagaagaaataatttaaatgaactgcttagccctgctctgaagaaccttttttggaattagattttagttgattttttaaagaatacagcccaacacttgttttttacattttaagagttgtagaagttgttcctaacttggggactggacctaccctctaggagggagttgttaatggggctcttttagcccactgatgcttacttaggccggagagcaggggacacggtgctaggttccctcgtgcagtgcctggtgctctcaaattgtctcaaaaggaccaagaggaaaagagtcggaggggtagaccctgcagccctgttgagaagaaagattccagtgaagttgctgatggtatggctgtggtctgggacttgcggtgtctcggcatacccctctcctcttcccacctcctcctggctatgttctgcagcctcccagagtagaaaactactttgttacttaaggttgttcaccttgtaccagtggttatttgagtttgttcctattacaccaatccttacttgaggtggttcggattacagtttccaaatgcattctgggattgcctattccagagagggtacagaaaaagcacacagatggcttgtctcaggagtgttgaatgtgtgccccgctgctgtctggggggatgggagtgggctctggggtcatatgtgaacatccccttggatgatttgcggttgcttagaataaaacttgctactagcaaaagaaaaaaaaaaaaaaaaaaaaaaaaaaaaaaaa

SEQ ID NO: 66 - protein, 495 aa, Homo sapiens
MAESAPARHRRKRRSTPLTSSTLPSQATEKSSYFQTTEISLWTVVAAIQAVEKKMESQAARLQSLEGRTGTAEKKLADCEKMAVEFGNQLEGKWAVLGTLLQEYGLLQRRLENVENLLRNRNFWILRLPPGSKGEAPKVSRSLENDGVCFTEQEWENLEDWQKELYRNVMESNYETLVSLKVLGQTEGEAELGTEMLGDLEEEGPGGAHPAGGVMIKQELQYTQEGPADLPGEFSCIAEEQAFLSPEQTELWGGQGSSVLLETGPGDSTLEEPVGSRVPSSSRTVGCPKQKSHRQVQLDQECGQGLKLKKDTSRPYECSECEITFRYKQQLATHLRSHSGWGSCTPEEPEESLRPRPRLKPQTKKAKLHQCDVCLRSFSCKVSLVTHQRCHLQEGPSAGQHVQERFSPNSLVALPGHIPWRKSRSSLICGYCGKSFSHPSDLVRHQRIHTGERPYSCTECEKSFVQKQHLLQHQKIHQRERGGLALEPGRPNGLL

SEQ ID NO: 67 - DNA, 1020 bp, Homo sapiens
atggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcagcaacagcaacagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtcgccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaa

SEQ ID NO: 68 - protein, 339 aa, Homo sapiens
MDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRRRNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 69 - DNA, 2240 bp, Homo sapiens
gtatttgtttcactgctttcaaccgcctgtgctggaggctcagaataagtcaatgggaggaggatttcagtcacagcagcaagcaagtctagtgaacagataagatgacatgctcagcaaaataacaacgaaaccagagggggaactctctggcatgcaagttcaaacacgactctacaactacggcagaaaaagagagagagagaaactaaaaatatatatatatcctatttttttcacagctatcagtttctttcactgagctttcctaaatttaagcctctagaaaataataaatacttggatatcttacctacaaacatggacagatgtgtgtatgcgctcattttagagaacttgaatttttttttttaaaggaaggtgtcaactttggcttttgagtgtttggcatggttacaatgccttaaaaaaacagatgagcagcttagctactaaccatgctgaccactgttcggaacgggattgaatcacagaaaaacagcaaatggctctctcttacagagtgtctgaacttcaaagcacaattcctgagcacattttgcagagcacctttgttcacgttatctcttctaactggtctggattacagacagaatcaataccagaggaaatgaaacagattgttgaggaacagggaaataaactgcactgggcagctcttctgatactcatggtgataatacccacaattggtggaaatacccttgttattctggctgtttcactggagaagaagctgcagtatgctactaattactttctaatgtccttggcggtggctgatttgctggttggattgtttgtgatgccaattgccctcttgacaataatgtttgaggctatgtggcccctcccacttgttctatgtcctgcctggttatttcttgacgttctcttttcaaccgcatccatcatgcatctctgtgccatttcagtggatcgttacatagccatcaaaaagccaatccaggccaatcaatataactcacgggctacagcattcatcaagattacagtggtgtggttaatttcaataggcattgccattccagtccctattaaagggatagagactgatgtggacaacccaaacaatatcacttgtgtgctgacaaaggaacgttttggcgatttcatgctctttggctcactggctgccttcttcacacctcttgcaattatgattgtcacctactttctcactatccatgctttacagaagaaggcttacttagtcaaaaacaagccacctcaacgcctaacatggttgactgtgtctacagttttccaaagggatgaaacaccttgctcgtcaccggaaaaggtggcaatgctggatggttctcgaaaggacaaggctctgcccaactcaggtgatgaaacacttatgcgaagaacatccacaattgggaaaaagtcagtgcagaccatttccaacgaacagagagcctcaaaggtcctagggattgtgtttttcctctttttgcttatgtggtgtcccttctttattacaaatataactttagttttatgtgattcctgtaaccaaactactctccaaatgctcctggagatatttgtgtggataggctatgtttcctcaggagtgaatcctttggtctacaccctcttcaataagacatttcgggatgcatttggccgatatatcacctgcaattaccgggccacaaagtcagtaaaaactctcagaaaacgctccagtaagatctacttccggaatccaatggcagagaactctaagtttttcaagaaacatggaattcgaaatgggattaaccctgccatgtaccagagtccaatgaggctccgaagttcaaccattcagtcttcatcaatcattctactagatacgcttctcctcactgaaaatgaaggtgacaaaactgaagagcaagttagttatgtatagcagaactggcagttgtcatcaaacataatgatgagtaagatgatgaatgagatgtaaatgtgccaagaatatattatataaagaattttatgtcatatatcaaatcatctctttaacctaagatgtaagtattaagaatatctaattttcctaatttggacaagattattccatgaggaaaataattttatatagctacaaatgaaaacaatccagcactctggttaaattttaaggtattcgaatgaaataaagtcaaatcaataaatttcaggctttaaaaaaaaaa

SEQ ID NO: 70 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEEQVSYV

SEQ ID NO: 71 - DNA, 2305 bp, Homo sapiens
ggcaacaggcagatttgcctgctgagggtggagacccacgagccgaggcctcctgcagtgttctgcacagcaaaccgcacgctatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgacggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacgtaagaacaaaaaaaaaaaa

SEQ ID NO: 72 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 73 - DNA, 6593 bp, Homo sapiens
atggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcagaaaaaaaaaaaaaaaaaa

SEQ ID NO: 74 - protein, 2102 aa, Homo sapiens
MDQSYVCALIAMMVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 75 - DNA, 50 bp, Artificial
  Synthetic
cggtggagtatggcaaggacagttgcatcatcaaagaaggagacgtgggg

SEQ ID NO: 76 - DNA, 50 bp, Artificial
  Synthetic
tgcaccagaaagagcagttcgcttttggggtggatacagagctgaactgc

SEQ ID NO: 77 - DNA, 50 bp, Artificial
  Synthetic
gcaaagccccaaacagagaacatccagatgctgggatcaccatatcgctg

SEQ ID NO: 78 - DNA, 50 bp, Artificial
  Synthetic
aagaaggagaccgtctggcatctggaggagtttggccaagccttttcctt

SEQ ID NO: 79 - DNA, 50 bp, Artificial
  Synthetic
gcaacaatgaaattaatggataccgtctgcccttggcccagaattgttat

SEQ ID NO: 80 - DNA, 50 bp, Artificial
  Synthetic
tggccaacatagctgtggacaaagccaacctggaaatcatgacaaagcgc

SEQ ID NO: 81 - DNA, 50 bp, Artificial
  Synthetic
tggaaacagttcctcggagtggagaggtttacacctgccaagtggagcac

SEQ ID NO: 82 - DNA, 50 bp, Artificial
  Synthetic
agacctaccacgtcaatatggcgggcaccaacccctacacaaccatcacg

SEQ ID NO: 83 - DNA, 50 bp, Artificial
  Synthetic
cagttctggaggatggttgcacgaaacacactggggaatggagcaaaaca

SEQ ID NO: 84 - DNA, 50 bp, Artificial
  Synthetic
ttcaggaatcggaatcctgtcgattagactggacagcttgtggcaagtga

SEQ ID NO: 85 - DNA, 50 bp, Artificial
  Synthetic
gggagaaaatggccagatgatgagctgcacatgtcttgggaacggaaaag

SEQ ID NO: 86 - DNA, 50 bp, Artificial
  Synthetic
gtgctatggcctctgggaccatgaggaggactcaccatccaaggagcatg

SEQ ID NO: 87 - DNA, 50 bp, Artificial
  Synthetic
gggtcgctcaaggcttaactccaacacgcaaggggagatgaagtttgagg

SEQ ID NO: 88 - DNA, 50 bp, Artificial
  Synthetic
ccacacatcagcacaactacgcagcgcctccctccactcggaaggactat

SEQ ID NO: 89 - DNA, 50 bp, Artificial
  Synthetic
gattctagctgtgagaacggggccttcttctgcaatgagtgtgactgccg

SEQ ID NO: 90 - DNA, 50 bp, Artificial
  Synthetic
cgaaacgccgaatataatcccaagcggtttgctgcggtaatcatgaggat

SEQ ID NO: 91 - DNA, 50 bp, Artificial
  Synthetic
cagcttcggagagttctgggattgtaccgcagctgcaaaatattgtatcc

SEQ ID NO: 92 - DNA, 50 bp, Artificial
  Synthetic
ggtggggagctgtgatgtgaagtttcctataaggttagaaggccttgtgc

SEQ ID NO: 93 - DNA, 50 bp, Artificial; identical to SEQ ID NO: 75
  Synthetic

SEQ ID NO: 94 - DNA, 50 bp, Artificial
  Synthetic
ggcaccagattgttacgatgagcaatgatgtttctgaggaggagagcgag

SEQ ID NO: 95 - DNA, 50 bp, Artificial
  Synthetic
accttggttcaccttatgaaccccgagtatgtccccaaaccgtgctgtgc

SEQ ID NO: 96 - DNA, 50 bp, Artificial
  Synthetic
ggtgggacgatgagactttgaaactatctcatgccagtgccttattaccc

SEQ ID NO: 97 - DNA, 50 bp, Artificial
  Synthetic
gcacagagactctgacctgtttttgttggacacccgtgtagtatgggcct

SEQ ID NO: 98 - DNA, 50 bp, Artificial
  Synthetic
gctcacctcttctttaccagaacggttctttgaccagcacattaacttct

SEQ ID NO: 99 - DNA, 50 bp, Artificial
  Synthetic
aaggccttaagctttggacccaagggaaaactgcatggagacgcatttcg

SEQ ID NO: 100 - DNA, 50 bp, Artificial
  Synthetic
cctgcttttaggagaccgaagtccgcagaacctgcctgtgtcccagcttg

SEQ ID NO: 101 - DNA, 50 bp, Artificial
  Synthetic
atgcttttgttcagggctgtgatcggcctggggaaataataaagcacgct

SEQ ID NO: 102 - DNA, 50 bp, Artificial
  Synthetic
cctttcttggctctgctgacactcgagcccacattccgtcacctgctcag

SEQ ID NO: 103 - DNA, 50 bp, Artificial
  Synthetic
tggctctctgcaaccagttctctgcatcacttgctgctgacacgccgacc

SEQ ID NO: 104 - DNA, 50 bp, Artificial
  Synthetic
gtgtgtttgtgattgtttgctctgagagttcccctgtcccctcccccttc

SEQ ID NO: 105 - DNA, 50 bp, Artificial
  Synthetic
gcgagtagatgaacctgcagcaagcagcgtttatggtgcttccttctccc

SEQ ID NO: 106 - DNA, 50 bp, Artificial
  Synthetic
agcaaagatacctccccgaaacagcggatccagaatctcagccgctttgt

SEQ ID NO: 107 - DNA, 50 bp, Artificial
  Synthetic
cactggccccacattcctcaactagtattatttgggctctgggcagcagc

SEQ ID NO: 108 - DNA, 50 bp, Artificial
  Synthetic
ggggcaatggaggaactaagcactactctaagccaagctgagggagtccg

SEQ ID NO: 109 - DNA, 50 bp, Artificial
  Synthetic
gacagaccactccagataccgaggctgcagatgatgtggggtcaaagagc

SEQ ID NO: 110 - DNA, 50 bp, Artificial
  Synthetic
aaaagcacttcaaggggtcaaagggcaaccagcttgggtgctacctcagt

SEQ ID NO: 111 - DNA, 50 bp, Artificial
  Synthetic
caaccagtccaaagggaaggttgctggataggcgatccagatctgggaaa

SEQ ID NO: 112 - DNA, 50 bp, Artificial
  Synthetic
caaaaccagcgggcttgaaagaatcctcggataaagtgtccagggtggct

SEQ ID NO: 113 - DNA, 50 bp, Artificial
  Synthetic
ttctttctgggtgatctggggatcacgccttgcccaagtgtgagattacc

SEQ ID NO: 114 - DNA, 50 bp, Artificial
  Synthetic
ttgatcctccccctcacatgagagtacctaccattcctccaaacctggca

SEQ ID NO: 115 - DNA, 50 bp, Artificial
  Synthetic
gcctcctcggcaagttccacttctttgaaatccaaagaaatgagcttgca

SEQ ID NO: 116 - DNA, 50 bp, Artificial
  Synthetic
ggaatcgacaaaaatcgcctgctaaagaaggatgcttctagcagtccagc

SEQ ID NO: 117 - DNA, 50 bp, Artificial
  Synthetic
gcttcctttgcaaatgaagatgggcaggttagcccaggaagcctgctctt

SEQ ID NO: 118 - DNA, 50 bp, Artificial
  Synthetic
cctgatcacgttgggggctacaaactggcagttgctacatttgcagggat

SEQ ID NO: 119 - DNA, 50 bp, Artificial
  Synthetic
ggaggagtttgttctggcagctcagaaatttggtcaggttacacccatgg

SEQ ID NO: 120 - DNA, 50 bp, Artificial
  Synthetic
cgagtcagtgctctgtctgtcgtgcgggacctggggttttttgggatcta

SEQ ID NO: 121 - DNA, 50 bp, Artificial
  Synthetic
caccaccactggacgagcagaacagggactgccagggtctgatggagaag

SEQ ID NO: 122 - DNA, 50 bp, Artificial
  Synthetic
gctcgaaactgggccaatcaaagatcagctttgggaagtgctcttgatcc

SEQ ID NO: 123 - DNA, 50 bp, Artificial
  Synthetic
cctaaagaccattgcacttcgtcgccgaaacgccgaatataatcccaagc

SEQ ID NO: 124 - DNA, 50 bp, Artificial; identical to SEQ ID NO: 91
  Synthetic

SEQ ID NO: 125 - DNA, 50 bp, Artificial; identical to SEQ ID NO: 92
  Synthetic

SEQ ID NO: 126 - DNA, 50 bp, Artificial; identical to SEQ ID NO: 75
  Synthetic

SEQ ID NO: 127 - DNA, 50 bp, Artificial
  Synthetic
ctgacgaaacagaagacccgaacggtgaaagccacgctaaaccctgtgtg

SEQ ID NO: 128 - DNA, 50 bp, Artificial
  Synthetic
ggacgtacctttcgcctcctcaaagcctctaagaatgggttgcaggccag

SEQ ID NO: 129 - DNA, 50 bp, Artificial
  Synthetic
ccaatggcctctttcctcccaaataaaccactggctttctctttgtcccc

SEQ ID NO: 130 - DNA, 50 bp, Artificial
  Synthetic
tgttctgcaatggacttgtcctgcatcgacttcagtgccttcgtggattt

SEQ ID NO: 131 - DNA, 50 bp, Artificial
  Synthetic
ccaccttctcctccaatctgcatgatgaatgcccttgtccgagctttaac

SEQ ID NO: 132 - DNA, 50 bp, Artificial
  Synthetic
ccctgtcgatcccttctgaggtatggcccatccaagacttttaggccatt

SEQ ID NO: 133 - DNA, 50 bp, Artificial
  Synthetic
ggtcactggagaatgatggcgtctgtttcaccgagcaggaatgggagaat

SEQ ID NO: 134 - DNA, 50 bp, Artificial
  Synthetic
cgaaatgggattaaccctgccatgtaccagagtccaatgaggctccgaag

SEQ ID NO: 135 - DNA, 50 bp, Artificial
  Synthetic
tccagggcaactctagcatcagagcaaaagccttgggtttctcgcattca

SEQ ID NO: 136 - DNA, 50 bp, Artificial
  Synthetic
ttttgcctatcctgacactcaccgccatcgcctgggacccaattatcttc

SEQ ID NO: 137 - DNA, 50 bp, Artificial
  Synthetic
ggaagagggagataacgctgtgctgcagtgcctcaaggggacctcagatg

SEQ ID NO: 138 - DNA, 50 bp, Artificial
  Synthetic
aacaaactgcacccactgaactccgcagctagcatccaaatcagcccttg

SEQ ID NO: 139 - DNA, 50 bp, Artificial
  Synthetic
gccgacacacaagctctgttgaggaatgaccaggtctatcagcccctccg

SEQ ID NO: 140 - DNA, 50 bp, Artificial
  Synthetic
ttcccctatggacacctcagctttggattcatgcaggacctccagcagaa

SEQ ID NO: 141 - DNA, 50 bp, Artificial
  Synthetic
aaaaaaaaaaaaaaaaaaaaaaaaaaaaaaaaaaaaaaaaaaaaaaaaaa

SEQ ID NO: 142 - DNA, 50 bp, Artificial
  Synthetic
ccactgcaaggaacaacaggccttattccactgctggggattgatgtgtg

SEQ ID NO: 143 - DNA, 50 bp, Artificial
  Synthetic
ccctgccataacccaataccaaacgcccctcttcgtctgatccgtcctaa

SEQ ID NO: 144 - DNA, 50 bp, Artificial; identical to SEQ ID NO: 123
  Synthetic

SEQ ID NO: 145 - DNA, 50 bp, Artificial; identical to SEQ ID NO: 91
  Synthetic

SEQ ID NO: 146 - DNA, 50 bp, Artificial; identical to SEQ ID NO: 92
  Synthetic

(19) United States
(12) Patent Application Publication
(10) Pub. No.: US 2011/0159492 A1
(43) Pub. Date: Jun. 30, 2011

(54) Diffuse Large B-Cell Lymphoma Markers and Uses Therefore

(76) Inventors: Lisa M. Rimsza, Tueson, AZ (US); Michael L. Leblanc, Seattle, WA (US); Joseph M. Unger, Seattle, WA (US)

(21) Appl. No.: 12/996,112

(22) PCT Filed: Jun. 4, 2009
(86) PCT No.: PCT/US09/46314

§ 371 Date: Mar. 11, 2011

(30) Foreign Application Priority Data
Jun. 4, 2008 (US) 61/131027

(51) Int. Cl. C12Q 1/68; G01N 33/574

(52) U.S. Cl. 435/6.11; 436/501

(57) ABSTRACT

The present invention provides methods and compositions for prognosing treatment outcome in DLBCL patients, diagnosing DLBCL and monitoring efficacy of DLBCL treatment.

### RELATED APPLICATIONS

This application claims priority to U.S. Provisional Patent Application Ser. No. 61/131,027, filed Jun. 4, 2008, which is incorporated by reference herein in its entirety.

### BACKGROUND

Cancer is a major cause of morbidity in the United States and in most other industrialized nations. For example, in 2007, the American Cancer Society Surveillance Research division estimated that 1,444,920 people were diagnosed with cancer and that 559,650 died from the disease. Cancer is responsible for nearly a quarter of all American deaths and is exceeded only by heart disease as a cause of mortality. Despite improved care and treatment, cancer mortality is rapidly increasing in the United States and is soon expected to become the leading cause of mortality in this country as it already is in Japan.

Cancers are characterized by abnormal cell division, growth, and/or differentiation. Their initial clinical manifestations are extremely heterogeneous, with over 70 types of cancer arising in virtually every organ and tissue of the body. Moreover, some of those similarly classified cancer types may represent multiple different molecular diseases. Unfortunately, some cancers may be virtually asymptomatic until late in the disease course, when treatment is more difficult, and prognosis grim. Thus there is a need for improved diagnosis and detection of cancer, especially at the initial stages, which allows for improved prognosis and better chances for survival.

Additionally, in about 4% of all patients diagnosed with cancer, the observed tumor is due to metastasis and the primary tumor origin is undetermined (see Hillen, Postgrad. Med. J., 76:690-693, 2000). Thus, a central goal of cancer biology is the identification of molecules or sets of molecules that are unique to specific human carcinomas, both for the development of diagnostics and drugs for the treatment of disease, as well as ultimately to understand the mechanistic basis of tissue-specific tumorigenesis. The identification of genes whose expression is uniquely characteristic of tumors of diverse anatomic origins remains a central challenge to the development of new cancer therapies

Treatment for cancer typically includes surgery, chemotherapy, and/or radiation therapy. Although nearly 50 percent of cancer patients can be effectively treated using these methods, the current therapies all induce serious side effects which diminish quality of life. The identification of novel therapeutic targets and diagnostic markers is desirable for improving the diagnosis, prognosis, and treatment of cancer patients.

With advances in high-density DNA microarray technology, it has become possible to screen tens of thousands genes at the same time to determine whether or not they are active in tumor tissues. “Gene expression profiling” is coined to describe such an approach. Like any cells, behavior of tumor cells is dictated by the expression of thousands of genes. Study of gene expression profiling therefore allows efficient identification of tumor biomarkers, drugable targets, classifiers of tumor subtypes and predictors of clinical outcome.

### SUMMARY OF THE INVENTION

In a first aspect, the present invention provides methods of prognosing an outcome of treatment for diffuse large B cell lymphoma (DLBCL) in a patient comprising obtaining a test sample from a patient with DLBCL; detecting a level of expression products of between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein a level of expression product of no more than sixteen genes in total is detected; and comparing an expression product level of the genes in the test sample with an expression product level of the genes in a control; wherein the expression product levels of the genes in the test sample compared to the expression product levels of the gene in a control is prognostic for an outcome of treatment for the patient with DLBCL if treated with combination chemotherapy.

In one preferred embodiment of this first aspect, the combination chemotherapy comprises a combination of cyclophsophamide, oncovorin, prednisone, and one or more chemotherapeutics selected from the group consisting of hydroxydaunorubicin, epirubicin, and motixantrone. This combination is known as (CHOP) or CHOP-like chemotherapy.

In another preferred embodiment of this first aspect, the combination chemotherapy comprises a combination of an anti-CD20 antibody and CHOP or CHOP-like chemotherapy.

In one preferred embodiments of this first aspect, the control comprises average expression product levels of the genes in a control patient population. In another preferred embodiment the method further comprises assessing an international prognostic index (IPI) for the patient in prognosing the treatment outcome. In various further preferred embodiments of the first aspect, of the invention, the expression product is selected from the group consisting of mRNA expression products and protein expression products.

In a second aspect, the present invention provides methods for prognosing an outcome of treatment for diffuse large B cell lymphoma (DLBCL) in a patient comprising obtaining a test sample from a patient with DLBCL; detecting a level of expression products one or more genes selected from the group consisting of GCET1, HLA-DQA1, HLA-DRB, HLA-DRA, ACTN1, COL3A1, PLAU, MYC, BCL6, LMO2, PDCD4, and SOD2; and comparing an expression product level of the one or more genes in the test sample with an expression product level of the one or more genes in a control; wherein the expression product levels of the one or more genes in the test sample compared to the expression product levels of the one or more genes in a control is prognostic for an outcome of treatment for the patient with DLBCL if treated with monoclonal antibody therapy together with combination chemotherapy.

In one preferred embodiment of the second aspect, the combination chemotherapy comprises CHOP or CHOP-like chemotherapy.

In another preferred embodiments of this second aspect, the control comprises average expression product levels of the one or more genes in a control patient population. In a further preferred embodiment the method further comprises assessing an international prognostic index (IPI) for the patient in prognosing the treatment outcome. In various further preferred embodiments of the second aspect, of the invention, the expression product is selected from the group consisting of mRNA expression product and protein expression product.

In a third aspect, the present invention provides methods of prognosticating an outcome of treatment for diffuse large B cell lymphoma (DLBCL) in a patient comprising: obtaining a test sample from a patient with DLBCL; detecting a level of expression products of between one and twelve genes selected from the group consisting of GCET1, HLA-DQA1, HLA-DRB, HLA-DRA, ACTN1, COL3A1, PLAU, MYC, BCL6, LMO2, PDCD4, and SOD2; determining an international prognostic index (IPI) score for the patient; and comparing an expression product level of the genes in the test sample with an expression product level of the genes in a control; wherein the expression product levels of the genes in the test sample compared to the expression product levels of the gene in a control, in combination with an IPI score for the patient, is prognostic for an outcome of treatment for the patient with DLBCL if treated with combination chemotherapy

In one preferred embodiment of this third aspect, the combination chemotherapy comprises a combination of cyclophsophamide, oncovorin, prednisone, and one or more chemotherapeutics selected from the group consisting of hydroxydaunorubicin, epirubicin, and motixantrone. This combination is known as (CHOP) or CHOP-like chemotherapy.

In another preferred embodiment of this third aspect, the combination chemotherapy comprises a combination of an anti-CD20 antibody and CHOP or CHOP-like chemotherapy. In a further preferred embodiment of this seventh aspect, the control comprises average expression product levels of the genes in a control patient population. In various further preferred embodiments of the aspect of the invention, the expression product is selected from the group consisting of mRNA expression products and protein expression products. In a further preferred embodiment, the expression product levels of the genes in the test sample compared to the expression product levels of the gene in a control, in combination with an IPI score of 4 to 5 for the patient, is prognostic for an outcome of treatment for the patient with DLBCL if treated with combination chemotherapy.

In a fourth aspect, the present invention provides methods for monitoring efficacy of treatment for diffuse large B cell lymphoma (DLBCL) in a patient comprising obtaining a test sample from a patient undergoing treatment for DLBCL; detecting a level of expression products one or more genes selected from the group consisting of GCET1, HLA-DQA1, HLA-DRB, HLA-DRA, ACTN1, COL3A1, PLAU, MYC, BCL6, LMO2, PDCD4, and SOD2; and comparing an expression product level of the one or more genes in the test sample with an expression product level of the one or more genes in a control; wherein the expression product levels of the one or more genes in the test sample compared to the expression product levels of the one or more genes in a control provides a measure of efficacy of treatment of the patient.

In a fifth aspect, the present invention provides methods for treating a patient with DLBCL, comprising or consisting of administering to the patient a pharmaceutical composition in an amount effective to alter expression product level of one or more genes selected from the group consisting of GCET1, HLA-DQA1, HLA-DRB, HLA-DRA, ACTN1, COL3A1, PLAU, MYC, BCL6, LMO2, PDCD4, and SOD2.

In a sixth aspect, the present invention provides compositions, comprising or consisting of reagents for detection of expression products of between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein the reagents are optionally detectably labeled. In various preferred embodiments, the reagents comprise or consist of nucleic acid probes, nucleic acid primers, antibodies, and/or aptamers.

In a seventh aspect, the present invention provides kits comprising one or more compositions of the present invention.

### BRIEF DESCRIPTION OF THE FIGURES

FIG. 1 shows overall survival in years for 3 representative genes in patients treated with CHOP versus R-CHOP according to gene expression levels. HLA-DRB is cut above and below 25%, BCL6 and C-MYC are cut at median. (A) CHOP treated cases, all IPI scores, Panel (i): HLA-DRB, Panel (ii): BCL6, Panel (iii): C-MYC (N=93). (B) R-CHOP cases, all IPI scores, HLA-DR, BCL6, and C-MYC (N=116).

FIG. 2. shows overall survival in years for patients treated with R-CHOP according to IPI score and expression levels of HLA-DRB and/or C-MYC. Cut point levels are above and below the median for both genes. Adverse gene level for HLA-DR is for expression below the median, while adverse gene level for C-MYC is for expression above the median. Panel (A) All IPI groups (N=116), either without the two adverse genes levels of high c-MYC and low HLA-DRB (n=88) or with high c-MYC and low HLA-DRB (n=28). Panel (B) Low IPI group (scores 0-2, N=72), either without the two adverse genes levels of high c-MYC and low HLA-DRB (n=61) or with high c-MYC and low HLA-DRB (n=11). Panel (C) High IPI group (scores 3-5, N=36), either without the two adverse genes levels of high c-MYC and low HLA-DRB (n=22) or with high c-MYC and low HLA-DRB (n=14). The combined number of cases in (B) and (C) are fewer than (A) due to several cases with missing IPI information.

FIG. 3. shows variable cut point analysis for HLA-DRB and C-MYC genes. Gene expression level on X-axis, log rank score on Y-axis, permutation p-value indicated. (A) HLA-DRB, (B) CMYC. The peaks in the log rank scores indicate the most significant cut-points in the data yielding the largest differences in overall survival.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

The present invention provides methods and compositions for prognosing treatment outcome in DLBCL patients, diagnosing DLBCL, monitoring efficacy of DLBCL treatment, and methods for treating DLBCL patients.

In a first aspect, the present invention provides methods of prognosing an outcome of treatment for diffuse large B cell lymphoma (DLBCL) in a patient comprising obtaining a test sample from a patient with DLBCL; detecting a level of expression products of between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein a level of expression product of no more than sixteen genes in total is detected; and comparing an expression product level of the genes in the test sample with an expression product level of the genes in a control; wherein the expression product levels of the genes in the test sample compared to the expression product levels of the gene in a control is prognostic for an outcome of treatment for the patient with DLBCL if treated with combination chemotherapy. By “outcome” it is meant prognosis of patient response to treatment in terms of overall survival (OS) or progression free survival. An individual who is at risk for poor outcome (shorter disease free survival, shorter progression free survival or shorter overall survival” relative to DLBCL patient population as a whole) is an individual in whom two or more genes selected from the group consisting of GCET1, HLA-DQA1, HLA-DRB, HLA-DRA, ACTN1, COL3A1, PLAU, MYC, BCL6, LMO2, PDCD4, and SOD2 are differentially expressed as compared to a suitable control, as discussed in more detail below. In a preferred embodiment, the outcome is measured in terms of a “hazard ratio” (the ratio of death rates for one patient group to another; provides likelihood of death at a certain time point). In another preferred embodiment, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. Thus, the invention further provides methods for making a treatment decision for a DLBCL patient, comprising carrying out the methods for prognosing a DLBCL patient according to the different aspects and embodiments of the present invention, and then weighing the results in light of other known clinical and pathological risk factors, in determining a course of treatment for the DLBCL patient. For example, a DLBCL patient that is shown by the methods of the invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation.

As used in all aspects of the present invention, the term “patient” or “subject” as used herein refers to mammals (e.g., humans and animals), most preferably humans.

As used in all aspects of the present invention, “Diffuse large B-cell lymphoma” or “DLBCL” as used herein, is a fast-growing, aggressive form of non-Hodgkin's lymphoma (NHL) which originates in centrocytes in the light zone of germinal centers. It is one of the most common types of NHL. Several types of DLBCL are known in the art, based on pathological studies and clinical staging procedures. For example, morphological variants include, but are not limited to, centroblastic DLBCL, immunoblastic DLBCL, anaplastic DLBCL, plasmablastic DLBCL, anaplastic lymphoma kinase-positive DLBCL, etc. Subtypes include, but are not limited to, mediastinal (thymic) large B-cell lymphoma, intravascular large B-cell lymphoma, T-cell/histiocyte-rich large B-cell lymphoma, lymphomatoid granulomatosis-type large B-cell lymphoma, primary effusion lymphoma, etc.

As used in all aspects of the present invention, the “test sample” comprises a biological specimen isolated from a patient suffering from DLBCL from which gene expression products can be obtained. Any suitable test sample can be used that is involved in the lymphoma (as lymphoma can occur anywhere in the body), including but not limited to a circulating fluid such as blood or lymph, or a fraction thereof, such as serum or plasma; synovial fluid, cerebrospinal fluid, interstitial fluid; urine, breast milk, saliva, sweat, tears, mucous, nipple aspirants, semen, vaginal fluid, pre-ejaculate and the like; a liquid in which cells are cultured in vitro such as a growth medium, or a liquid in which a cell sample is homogenized, such as a buffer; tissue, biopsied tissue, tissue sections, cultured cells, surgically resected tumor sample, etc.; and frozen sections or formalin fixed sections taken for histological purposes. In a preferred embodiment, the test sample comprises biopsied tissue from the DLBCL patient. In a further preferred embodiment, the test sample comprises formalin fixed tissue, such as a formalin fixed biopsied tissue from the DLBCL patient. In one preferred embodiment, the nucleic acid and/or polypeptide expression products are derived from one of the above types of control samples using standard techniques in the art. Such nucleic acid and/or polypeptide expression products may be isolated, partially isolated, or non-purified, such as when in situ detection methods are employed, as discussed in more detail below. The term “isolated,” as used herein, with respect to nucleic acids (such as DNA or RNA) and polypeptides means substantially free of cellular material, viral material, culture medium when produced by recombinant DNA techniques, or chemical precursors or other chemicals when chemically synthesized. Nucleic acid samples used in the methods of the invention may be prepared by any suitable method or process. Methods of isolating mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I Theory and Nucleic Acid Preparation, Tijssen, (1993) (editor) Elsevier Press. Such expression products may comprise or consist of mRNA, cDNA synthesized from mRNA expression products, DNA amplified from the cDNA, and RNA transcribed from the amplified DNA. One of skill in the art would appreciate that it is desirable to inhibit or destroy RNase present in homogenates before homogenates can be used. In a preferred embodiment the nucleic acid sample is simply prepared by treating the sample with lysis reagent, and more preferred, by the additional step of heating at 95° C., without extraction or purification of the nucleic acids from the sample.

As used in all aspects of the invention, the gene “expression products” whose level is to be measured may be mRNA and/or protein. As noted above, an “mRNA expression product” can be measured by measurement of cDNA generated from the mRNA in, for example, a reverse transcription-PCR reaction or other suitable amplification reaction.

As used herein for all aspects of the invention, the term “expression product level” refers to the measurable expression level of a given mRNA or protein expression product. The expression product level is determined by methods well known in the art, as described in more detail below. The term “differentially expressed” or “differential expression” refers to an increase or decrease in the measurable expression level of a given expression product. As used herein, “differentially expressed” or “differential expression” means the difference in the level of expression of an expression product is significant (e.g. p≦0.05), which can be at least a 1.2-fold, or, in various preferred embodiments, at least a 1.4-fold, 1.5-fold, 2-fold, 5-fold, 10-fold, 20-fold, 50-fold or greater difference in the expression product level between the test sample and appropriate control. In one embodiment, expression product level is determined in two test samples used for comparison, both of which are compared to expression product levels from the same housekeeper gene, and then subsequently compared to a suitable reference standard. Absolute quantification of the level of expression of an expression product may be accomplished, if desired, by any suitable technique, including but not limited to providing a known concentration(s) of one or more control expression products, generating a standard curve based on the amount of the control expression products and extrapolating the expression level of the “unknown” expression product from the intensities of the unknown (using standard detection assays) with respect to the standard curve.

Detecting an expression product level in any aspect of the present invention can be accomplished using any assays for measuring nucleic acid or protein levels, including but not limited to Northern blotting, nuclease protection assays, reverse transcription-polymerase chain reaction (RT-PCR), in situ hybridization, bDNA, sequencing, differential display, immunoblotting, Western blotting, enzyme-linked immunosorbent assays (ELISA), ligand binding assays, immunohistochemical assays (qualitative and quantitative), and immunocytochemical assays. In one preferred embodiment, the detection step is carried out using an array or chip-based method, as is known to those of skill in the art. In one preferred embodiment mRNA expression product levels are measured using a quantitative nuclease protection assay, qNPA, where the sample (including but not limited to formalin fixed paraffin-embedded tissue, (FFPE)) is treated with a lysis reagent, and nuclease protection probes are added and permitted to hybridize to target oligonucleotides in the sample. Nuclease S1 is then added to hydrolyze excess nuclease protection probe and unhybridized oligonucleotides; base is added and heated to dissociate the target gene oligonucleotide to nuclease protection probe hybrids, and the mixture is transferred onto an array where the nuclease protection probes are captured and quantified using a detection probe. Quantitative nuclease protection arrays (qNPA) and probes as described in U.S. Pat. Nos. 6,232,066 and 6,238,869 are preferably employed.

As used in all aspects of the invention, “combination chemotherapy” refers to the combination of any two or more chemotherapeutic drugs used in the field of chemotherapy to treat tumors, such as DLBCL. In one preferred embodiment, the combination chemotherapy comprises a combination of two or more of cyclophosphamide, hydroxydaunorubicin (also known as doxorubicin or adriamycin), oncovorin (vincristine) and prednisone. In another preferred embodiment, the combination chemotherapy comprises a combination of cyclophsophamide, oncovorin, prednisone, and one or more chemotherapeutics selected from the group consisting of hydroxydaunorubicin, epirubicin, and motixantrone. In a further preferred embodiment, the combination chemotherapy comprises a combination of each of cyclophosphamide, hydroxydaunorubicin, oncovorin, and prednisone, referred to as “CHOP” chemotherapy. In another embodiment, the combination therapy comprises CHOP-like chemotherapy. Examples of CHOP-like chemotherapy include, but are not limited to, CEOP (CHOP in which hydroxydaunorubicin is replaced with epirubicin) and CNOP (CHOP in which hydroxydaunorubicin is replaced with mitoxantrone, which is also known as novantrone).

In another preferred embodiment of this first aspect, the combination chemotherapy further comprises monoclonal antibody therapy. Any suitable monoclonal antibody therapy for use in treating tumors can be used. In one especially preferred embodiment, the monoclonal antibody therapy comprises anti-CD20 monoclonal antibody therapy. An “anti-CD20 antibody” as used herein is any antibody that is capable of binding to the CD20 epitope. The anti-CD20 antibody may be optionally radiolabeled, for example, with an isotope that emits alpha (α), beta (β) or gamma (γ) rays. Preferred embodiments of such anti-CD20 antibodies include, but are not limited to, rituximab (RITUXAN®). Preferred embodiments of such anti-CD20 radiolabeled antibodies that are commercially available include, but are not limited to, ibritumomab tiuxetan (ZEVALIN®) and tositumomab (BEXXAR®). In a most preferred embodiment, the anti-CD20 antibody is rituximab.

The present invention allows prognostication of patients with DLBCL that are treated with combination of CHOP therapy (or CHOP-like therapy) optionally with anti-CD20 antibody immunotherapy. Any combination of CHOP (or CHOP-like therapy) and anti-CD20 antibody may be studied. Preferred embodiments of such combinations include, CHOP in combination with rituximab (R-CHOP), CEOP in combination with rituximab (R-CEOP), CNOP in combination with rituximab (R-CNOP), ibritumomab in combination with CHOP (I-CHOP), ibritumomab in combination with CEOP (I-CEOP), ibritumomab in combination with CNOP (I-CNOP), tositumomab in combination with CHOP (T-CHOP), tositumomab in combination with CEOP (T-CEOP), and tositumomab in combination with CNOP (T-CNOP). In a most preferred embodiment, the present invention is directed to prognostication of DLBCL patients that are under R-CHOP therapy.

As used in all aspects of the present invention, the “control” can be any reference standard suitable to provide a comparison to the expression products in the test sample. In one preferred embodiment, the control comprises obtaining a “control sample” from which expression product levels are detected and compared to the expression product levels from the test sample. Such a control sample may comprise any suitable sample, including but not limited to a sample from a control DLBCL patient (can be stored sample or previous sample measurement) with a known outcome; normal tissue or cells isolated from a subject, such as a normal patient or the DLBCL patient, cultured primary cells/tissues isolated from a subject such as a normal subject or the DLBCL patient, adjacent normal cells/tissues obtained from the same organ or body location of the DLBCL patient, a tissue or cell sample isolated from a normal subject, or a primary cells/tissues obtained from a depository (for example, Novartis database depository with the GEO Accession No.: GSE1133). In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients (such as DLBCL patients), or a set of patients with a certain outcome (for example, survival for one, two, three, four years, etc.) or receiving a certain treatment (for example, CHOP or R-CHOP). It will be understood by those of skill in the art that such control samples and reference standard expression product levels can be used in combination as controls in the methods of the present invention. In one preferred embodiment, the control may comprise normal or non-cancerous cell/tissue sample. In another preferred embodiment, the control may comprise an expression level for a set of patients, such as a set of (e.g.) DLBCL patients, or for a set of DLBCL patients receiving a certain treatment (e.g. CHOP or R-CHOP as discussed below) or for a set of patients with one outcome versus another outcome. In the former case the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, for example, CHOP or R-CHOP, and cells from patients having benign lymphoma. In another preferred embodiment, the control may also comprise a measured value for example, average level of expression of a particular gene in a population compared to the level of expression of a housekeeping gene in the same population. Such a population may comprise normal subjects, patients with DLBCL who have not undergone any treatment (i.e., treatment naïve), DLBCL patients undergoing CHOP therapy, DLBCL patients undergoing R-CHOP therapy or patients having benign lymphoma. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another preferred embodiment, the control may comprise expression product levels grouped as percentiles within or based on a set of patient samples, such as all patients with DLBCL. In one embodiment a control expression product level is established wherein higher or lower levels of expression product relative to, for instance, a particular percentile, are used as the basis for predicting outcome. In another preferred embodiment, a control expression product level is established using expression product levels from DLBCL control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The methods of this first aspect of the invention comprise detecting a level of expression products of between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein a level of expression product of no more than 16 genes (including any control genes, such as housekeeping genes to normalize expression) are detected for prognosing DLBCL. These genes and their NCBI database accession numbers (for mRNA and polypeptide expression products) are provided below in Table 1, together with other genes assessed in the examples that follow. The sequence identifiers used herein for these genes are as follows:
1. BCL6: SEQ ID NO:1 (nucleic acid) and SEQ ID NO:2 (polypeptide)
2. GCET1 SEQ ID NO:3 (nucleic acid) and SEQ ID NO:4 (polypeptide)
3. PLAU SEQ ID NO:5 (nucleic acid) and SEQ ID NO:6 (polypeptide)
4. MYC SEQ ID NO:7 (nucleic acid) and SEQ ID NO:8 (polypeptide)
5. HLA-DQA1 SEQ ID NO:9 (nucleic acid) and SEQ ID NO:10 (polypeptide)
6. HLA-DRA SEQ ID NO:11 (nucleic acid) and SEQ ID NO:12 (polypeptide)
7. HLA-DRB SEQ ID NO:13 (nucleic acid) and SEQ ID NO:14 (polypeptide)
8. ACTN1 SEQ ID NO:15 (nucleic acid) and SEQ ID NO:16 (polypeptide)
9. COL3A1 SEQ ID NO:17 (nucleic acid) and SEQ ID NO:18 (polypeptide)
10. LMO2 SEQ ID NO:19 (nucleic acid) and SEQ ID NO:20 (polypeptide)
11. PDCD4 SEQ ID NO:21 (nucleic acid) and SEQ ID NO:22 (polypeptide)
12. SOD2 SEQ ID NO:23 (nucleic acid) and SEQ ID NO:24 (polypeptide)

In various preferred embodiments of this first aspect, the methods may comprise detecting a level of expression products of between 2, 3, 4, 5, 6, 7, 8, 9, 10, 11, or all 12 of the recited genes. In various other preferred embodiments of this first aspect, a level of expression product of no more than 16, 15, 14, 13, 12, 11, 10, 9, 8, 7, 6, 5, 4, 3, or 2 genes in total (including control genes) is detected for prognosing DLBCL. Any combination of two or more of the recited genes can be used in the methods of the invention. In one preferred embodiment of this first aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is detected. In a further preferred embodiment, at least one of the genes selected is MYC, HLA-DRB, or PDCD4, wherein elevated level of expression of MYC or PDCD4 is indicative of poor overall survival. In another preferred embodiment, the two or more genes comprise two or more of HLA-DRB, HLA-DRA, HLA-DQA1, BCL6, ACTN1, COL3A1, LMO2, or PLAU, wherein reduced level of expression the two or more genes, is indicative of poor overall survival. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2, wherein a reduced level of expression of HLA-DRB, HLA-DRA, PLAU, BCL6, ACTN1, and LMO2 is indicative of poor overall survival, and elevated level of expression of MYC or PDCD4 is indicative of poor overall survival. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. Each of these embodiments is particularly preferred for prognosing an outcome of R-CHOP therapy on a DLBCL patient. In another preferred embodiment, the two or more genes comprise two or more of MYC, HLA-DRB, HLA-DQA1, and PLAU, as differential expression of these genes is found herein to be associated with poor prognosis and/or survival outcome in DLBCL patients undergoing CHOP or R-CHOP therapy. All of these embodiments can be combined with the preferred embodiments above in which a level of expression product of no more than 16, 15, 14, 13, 12, 11, 10, 9, 8, 7, 6, 5, 4, 3, or 2 genes in total (including control genes) is detected for prognosing DLBCL, unless the context clearly dictates otherwise.

In a second aspect, the present invention provides methods for prognosing an outcome of treatment for diffuse large B cell lymphoma (DLBCL) in a patient comprising: obtaining a test sample from a patient with DLBCL; detecting a level of expression products one or more genes selected from the group consisting of GCET1, HLA-DQA1, HLA-DRB, HLA-DRA, ACTN1, COL3A1, PLAU, MYC, BCL6, LMO2, PDCD4, and SOD2; and comparing an expression product level of the one or more genes in the test sample with an expression product level of the one or more genes in a control; wherein the expression product levels of the one or more genes in the test sample compared to the expression product levels of the one or more genes in a control is prognostic for an outcome of treatment for the patient with DLBCL if treated with monoclonal antibody therapy together with combination chemotherapy. This second aspect of the invention is thus specific for prognosing a DLBCL patient outcome upon treatment with a combination of monoclonal antibody therapy and combination chemotherapy.

In this second aspect, all common terms are defined as above in the first aspect of the invention except where the context clearly indicates otherwise, and all embodiments of the first aspect of the invention can be used in this second and other aspects of the invention unless the context clearly indicates otherwise. In this second aspect, any suitable monoclonal antibody therapy for use in treating tumors can be used. In one especially preferred embodiment, the monoclonal antibody therapy comprises anti-CD20 monoclonal antibody therapy. An “anti-CD20 antibody” as used herein is any antibody that is capable of binding to the CD20 epitope. The anti-CD20 antibody may be optionally radiolabeled, for example, with an isotope that emits alpha (α), beta (β) or gamma (γ) rays. Preferred embodiments of such anti-CD20 antibodies include, but are not limited to, rituximab (RITUXAN®). Preferred embodiments of such anti-CD20 radiolabeled antibodies that are commercially available include, but are not limited to, ibritumomab tiuxetan (ZEVALIN®) and tositumoma. Any suitable combination chemotherapy can be used as described above. In one preferred embodiment, the combination chemotherapy comprises a combination of two or more of cyclophosphamide, hydroxydaunorubicin (also known as doxorubicin or adriamycin), oncovorin (vincristine) and prednisone. In another preferred embodiment, the combination chemotherapy comprises a combination of cyclophsophamide, oncovorin, prednisone, and one or more chemotherapeutics selected from the group consisting of hydroxydaunorubicin, epirubicin, and motixantrone. In a further preferred embodiment, the combination chemotherapy comprises a combination of each of cyclophosphamide, hydroxydaunorubicin, oncovorin, and prednisone, referred to as “CHOP” chemotherapy. In another embodiment, the combination therapy comprises CHOP-like chemotherapy. Examples of CHOP-like chemotherapy include, but are not limited to, CEOP (CHOP where hydroxydaunorubicin is replaced with epirubicin) and CNOP (CHOP where hydroxydaunorubicin replaced with mitoxantrone, which is also known as novantrone).

The methods of this second aspect of the invention comprise detecting a level of expression products of at least one gene selected from the group consisting of GCET1, HLA-DQA1, HLA-DRB, HLA-DRA, ACTN1, COL3A1, PLAU, MYC, BCL6, LMO2, PDCD4, and SOD2. These genes and their NCBI database accession numbers are provided below in Table 1, together with other genes assessed in the examples that follow. In various preferred embodiments of this second aspect, the methods may comprise detecting a level of expression products of between 1, 2, 3, 4, 5, 6, 7, 8, 9, 10, 11, or all 12 of the recited genes. Any combination of two or more of the recited genes can be used in the methods of the invention. In one preferred embodiment of this second aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is detected. In a further preferred embodiment, at least one of the genes selected is MYC, HLA-DRB, or PDCD4, wherein elevated level of expression of MYC or PDCD4 is indicative of poor overall survival. In another preferred embodiment, the two or more genes comprise two or more of HLA-DRB, HLA-DRA, HLA-DQA1, BCL6, ACTN1, COL3A1, LMO2, or PLAU, wherein reduced level of expression the two or more genes, is indicative of poor overall survival. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2, wherein a reduced level of expression of HLA-DRB, HLA-DRA, PLAU, BCL6, ACTN1, and LMO2 is indicative of poor overall survival, and elevated level of expression of MYC or PDCD4 is indicative of poor overall survival. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. Each of these embodiments is particularly preferred for prognosing an outcome of R-CHOP therapy on a DLBCL patient. In another preferred embodiment, the two or more genes comprise two or more of MYC, HLA-DRB, HLA-DQA1, and PLAU, as differential expression of these genes is found herein to be associated with poor prognosis and/or survival outcome in DLBCL patients undergoing CHOP or R-CHOP therapy. All of these embodiments can be combined with preferred embodiments in which a level of expression product of no more than 16, 15, 14, 13, 12, 11, 10, 9, 8, 7, 6, 5, 4, 3, or 2 genes in total (including control genes) is detected for prognosing DLBCL, unless the context clearly dictates otherwise.

In another embodiment of any aspect of the present invention, the method further comprises assessing an international prognostic index (IPI) for the patient in prognosticating the treatment outcome. Techniques and methodology for calculation of IPI to assign risk are known in the art and are discussed in the examples that follow. One point is assigned for each of the following risk factors: (1) age greater than 60 years; (2) stage III or IV disease; (3) elevated serum LDH; (4) ECOG/Zubrod performance status of 2 (Symptomatic, <50% in bed during the day), 3 (Symptomatic, >50% in bed, but not bedbound), or 4 (Bedbound); and (5) more than 1 extranodal site. The IPI score is determined by summing the total number of points. While the IPI has been a useful clinical tool for lymphoma patient risk stratification, it was developed prior to the use of monoclonal antibody therapy in DLBCL patients. For example, rituximab together with combination chemotherapy has dramatically improved the outcomes of DLBCL patients, and thus new methods for patient risk stratification are necessary.

In a further embodiment, the method further comprises assessing chromosomal alterations in the DLBCL patient, such as gains involving 3 p11-p12 (correlated with poor outcome), c-myc translocations, or other chromosomal alterations.

In a third aspect, the present invention provides methods of prognosticating an outcome of treatment for diffuse large B cell lymphoma (DLBCL) in a patient comprising: obtaining a test sample from a patient with DLBCL; detecting a level of expression products of between one and twelve genes selected from the group consisting of GCET1, HLA-DQA1, HLA-DRB, HLA-DRA, ACTN1, COL3A1, PLAU, MYC, BCL6, LMO2, PDCD4, and SOD2; determining an IPI score for the patient; and comparing an expression product level of the genes in the test sample with an expression product level of the genes in a control; wherein the expression product levels of the genes in the test sample compared to the expression product levels of the gene in a control, in combination with an IPI score for the patient, is prognostic for an outcome of treatment for the patient with DLBCL if treated with combination chemotherapy.

In this third aspect, all common terms are defined as above in the first and second aspects of the invention except where the context clearly indicates otherwise, and all embodiments of the first and second aspects of the invention can be used in this third (and other) aspects of the invention unless the context clearly indicates otherwise. In this third aspect, any suitable monoclonal antibody therapy for use in treating tumors can be used. In one especially preferred embodiment, the monoclonal antibody therapy comprises anti-CD20 monoclonal antibody therapy. An “anti-CD20 antibody” as used herein is any antibody that is capable of binding to the CD20 epitope. The anti-CD20 antibody may be optionally radiolabeled, for example, with an isotope that emits alpha (α), beta (β) or gamma (γ) rays. Preferred embodiments of such anti-CD20 antibodies include, but are not limited to, rituximab (RITUXAN®). Preferred embodiments of such anti-CD20 radiolabeled antibodies that are commercially available include, but are not limited to, ibritumomab tiuxetan (ZEVALIN®) and tositumoma. Any suitable combination chemotherapy can be used as described above. In one preferred embodiment, the combination chemotherapy comprises a combination of two or more of cyclophosphamide, hydroxydaunorubicin (also known as doxorubicin or adriamycin), oncovorin (vincristine) and prednisone. In another preferred embodiment, the combination chemotherapy comprises a combination of cyclophsophamide, oncovorin, prednisone, and one or more chemotherapeutics selected from the group consisting of hydroxydaunorubicin, epirubicin, and motixantrone. In a further preferred embodiment, the combination chemotherapy comprises a combination of each of cyclophosphamide, hydroxydaunorubicin, oncovorin, and prednisone, referred to as “CHOP” chemotherapy. In another embodiment, the combination therapy comprises CHOP-like chemotherapy. Examples of CHOP-like chemotherapy include, but are not limited to, CEOP (CHOP where hydroxydaunorubicin is replaced with epirubicin) and CNOP (CHOP where hydroxydaunorubicin replaced with mitoxantrone, which is also known as novantrone).

The methods of this third aspect of the invention comprise detecting a level of expression products of at least one gene selected from the group consisting of GCET1, HLA-DQA1, HLA-DRB, HLA-DRA, ACTN1, COL3A1, PLAU, MYC, BCL6, LMO2, PDCD4, and SOD2. These genes and their NCBI database accession numbers are provided below in Table 1, together with other genes assessed in the examples that follow. In various preferred embodiments of this third aspect, the methods may comprise detecting a level of expression products of between 1, 2, 3, 4, 5, 6, 7, 8, 9, 10, 11, or all 12 of the recited genes. Any combination of two or more of the recited genes can be used in the methods of the invention. In one preferred embodiment of this third aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is detected. In a further preferred embodiment, at least one of the genes selected is MYC, HLA-DRB, or PDCD4, wherein elevated level of expression of MYC or PDCD4 is indicative of poor overall survival. In another preferred embodiment, the two or more genes comprise two or more of HLA-DRB, HLA-DRA, HLA-DQA1, BCL6, ACTN1, COL3A1, LMO2, or PLAU, wherein reduced level of expression the two or more genes, is indicative of poor overall survival. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2, wherein a reduced level of expression of HLA-DRB, HLA-DRA, PLAU, BCL6, ACTN1, and LMO2 is indicative of poor overall survival, and elevated level of expression of MYC or PDCD4 is indicative of poor overall survival. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. Each of these embodiments is particularly preferred for prognosing an outcome of R-CHOP therapy on a DLBCL patient. In another preferred embodiment, the two or more genes comprise two or more of MYC, HLA-DRB, HLA-DQA1, and PLAU, as differential expression of these genes is found herein to be associated with poor prognosis and/or survival outcome in DLBCL patients undergoing CHOP or R-CHOP therapy. All of these embodiments can be combined with preferred embodiments in which a level of expression product of no more than 16, 15, 14, 13, 12, 11, 10, 9, 8, 7, 6, 5, 4, 3, or 2 genes in total (including control genes) is detected for prognosing DLBCL, unless the context clearly dictates otherwise.

In a further preferred embodiment, the expression product levels of the genes in the test sample compared to the expression product levels of the gene in a control, in combination with an IPI score of 4 to 5 for the patient, is prognostic for an outcome of treatment for the patient with DLBCL if treated with combination chemotherapy. As shown in the examples that follow, the combination of either adverse HLA-DRB or adverse c-Myc with an adverse IPI score of 4 to 5, results in the prognosis of a survival outcome of 20%, whereas an IPI score of 4 to 5 predicts 40% survival. Thus, the methods of the invention greatly improve over existing DLBCL patient stratification methods.

In a fourth aspect, the present invention provides methods for monitoring efficacy of treatment for diffuse large B cell lymphoma (DLBCL) in a patient comprising obtaining a test sample from a patient undergoing treatment for DLBCL; detecting a level of expression products one or more genes selected from the group consisting of GCET1, HLA-DQA1, HLA-DRB, HLA-DRA, ACTN1, COL3A1, PLAU, MYC, BCL6, LMO2, PDCD4, and SOD2; and comparing an expression product level of the one or more genes in the test sample with an expression product level of the one or more genes in a control; wherein the expression product levels of the one or more genes in the test sample compared to the expression product levels of the one or more genes in a control provides a measure of efficacy of treatment of the patient. All embodiments of other aspects disclosed herein apply to this aspect as well unless the context clearly dictates otherwise. In various preferred embodiments of this fourth aspect, the methods may comprise detecting a level of expression products of between 2, 3, 4, 5, 6, 7, 8, 9, 10, 11, or all 12 of the recited genes. In various other preferred embodiments of this third aspect, a level of expression product of no more than 16, 15, 14, 13, 12, 11, 10, 9, 8, 7, 6, 5, 4, 3, or 2 genes in total (including control genes) is detected for prognosing DLBCL. Any combination of two or more of the recited genes can be used in the methods of the invention. In one preferred embodiment of this fourth aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is detected. In a further preferred embodiment, at least one of the genes selected is MYC, HLA-DRB, or PDCD4, wherein elevated level of expression of MYC or PDCD4 is indicative of poor overall survival. In another preferred embodiment, the two or more genes comprise two or more of HLA-DRB, HLA-DRA, HLA-DQA1, BCL6, ACTN1, COL3A1, LMO2, or PLAU, wherein reduced level of expression the two or more genes, is indicative of poor overall survival. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2, wherein a reduced level of expression of HLA-DRB, HLA-DRA, PLAU, BCL6, ACTN1, and LMO2 is indicative of poor overall survival, and elevated level of expression of MYC or PDCD4 is indicative of poor overall survival. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. Each of these embodiments is particularly preferred for prognosing an outcome of R-CHOP therapy on a DLBCL patient. In another preferred embodiment, the two or more genes comprise two or more of MYC, HLA-DRB, HLA-DQA1, and PLAU, as differential expression of these genes is found herein to be associated with poor prognosis and/or survival outcome in DLBCL patients undergoing CHOP or R-CHOP therapy. All of these embodiments can be combined with the preferred embodiments above in which a level of expression product of no more than 16, 15, 14, 13, 12, 11, 10, 9, 8, 7, 6, 5, 4, 3, or 2 genes in total (including control genes) is detected for monitoring efficacy of DLBCL treatment, unless the context clearly dictates otherwise.

In a fifth aspect, the present invention provides methods for treating a patient with DLBCL, comprising or consisting of administering to the patient a pharmaceutical composition in an amount effective to alter expression product level of one or more genes selected from the group consisting of GCET1, HLA-DQA1, HLA-DRB, HLA-DRA, ACTN1, COL3A1, PLAU, MYC, BCL6, LMO2, PDCD4, and SOD2 An example of a modulator could comprise a new therapeutic regimen over an existing regimen, for example, the addition of anti-CD20 antibody immunotherapy on top of CHOP chemotherapy. All embodiments of other aspects disclosed herein apply to this aspect as well unless the context clearly dictates otherwise. In various preferred embodiments of this fifth aspect, the methods may comprise altering expression product level of between 2, 3, 4, 5, 6, 7, 8, 9, 10, 11, or all 12 of the recited genes. The method may comprise alteration of expression product level by any combination of two or more of the recited genes. Such alteration may comprise up-regulation (for example, by gene therapy, protein therapy, or cell therapy), or down-regulation (for example, by use of antisense or siRNA inhibitors, small molecule inhibitors, etc.). In one preferred embodiment of this fifth aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is altered. In a further preferred embodiment, at least one of the genes whose expression product is altered is MYC, HLA-DRB, or PDCD4, wherein elevated level of expression of MYC or PDCD4 is indicative of poor overall survival, and thus down-regulation of expression product levels is carried out. In another preferred embodiment, the two or more genes whose expression product is altered comprise two or more of HLA-DRB, HLA-DRA, HLA-DQA1, BCL6, ACTN1, COL3A1, LMO2, or PLAU, wherein reduced level of expression the two or more genes, is indicative of poor overall survival, and thus increases in expression level are carried out. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. In another preferred embodiment, the two or more genes comprise two or more of MYC, HLA-DRB, HLA-DQA1, and PLAU.

In another aspect, the invention further includes methods of screening for an agent capable of modulating the outcome of DLBCL in a subject, comprising contacting a tumor cell to the agent; and detecting the expression level of one or more genes selected from the group consisting of GCET1, HLA-DQA1, HLA-DRB, HLA-DRA, ACTN1, COL3A1, PLAU, MYC, BCL6, LMO2, PDCD4, and SOD2 in said tumor cell. All embodiments of other aspects disclosed herein apply to this aspect as well unless the context clearly dictates otherwise. In various preferred embodiments of this third aspect, the methods may comprise detecting a level of expression products of between 2, 3, 4, 5, 6, 7, 8, 9, 10, 11, or all 12 of the recited genes. In various other preferred embodiments of this aspect, a level of expression product of no more than 16, 15, 14, 13, 12, 11, 10, 9, 8, 7, 6, 5, 4, 3, or 2 genes in total (including control genes) is detected for prognosing DLBCL. Any combination of two or more of the recited genes can be used in the methods of the invention In one preferred embodiment of this aspect of the invention, a level of expression products from between two and eleven genes selected from the group consisting of GCET1, HLA-DQA1, HLA-DRB, ACTN1, COL3A1, PLAU, MYC, BCL6, LMO2, PDCD4, and SOD2 is detected. In a further preferred embodiment, at least one of the genes selected is MYC or PDCD4, wherein elevated level of expression of MYC or PDCD4 is indicative of poor overall survival. In another preferred embodiment, the two or more genes comprise two or more of HLA-DRB, HLA-DRA, HLA-DQA1, BCL6, ACTN1, COL3A1, LMO2, or PLAU, wherein reduced level of expression the two or more genes, is indicative of poor overall survival. In various further preferred embodiments, the at least two genes comprise a combination of MYC and one or more of HLA-DRB, HLA-DRA, PLAU, BCL6, ACTN1, and LMO2; or a combination of PDCD4 and one or more of HLA-DRB, PLAU, BCL6, ACTN1, and LMO2, wherein a reduced level of expression of HLA-DRB, HLA-DRA, PLAU, BCL6, ACTN1, and LMO2 is indicative of poor overall survival, and elevated level of expression of MYC or PDCD4 is indicative of poor overall survival. In various further preferred embodiments, the two or more genes comprise 2, 3, 4, 5, 6, 7, or 8 of MYC, HLA-DRB, HLA-DRA, PLAU, BCL6, ACTN1, LMO2, and PDCD4. Each of these embodiments is particularly preferred for prognosing an outcome of R-CHOP therapy on a DLBCL patient. In another preferred embodiment, the two or more genes comprise two or more of MYC, HLA-DRB, HLA-DQA1, and PLAU, as differential expression of these genes is found herein to be associated with poor prognosis and/or survival outcome in DLBCL patients undergoing CHOP or R-CHOP therapy. All of these embodiments can be combined with the preferred embodiments above in which a level of expression product of no more than 16, 15, 14, 13, 12, 11, 10, 9, 8, 7, 6, 5, 4, 3, or 2 genes in total (including control genes) is detected for monitoring efficacy of DLBCL treatment, unless the context clearly dictates otherwise.

In one preferred embodiment of the methods of all of the aspects and embodiments of the invention, detection or mRNA expression product level comprises the use of oligonucleotide probes that are homologous to the mRNA to be detected. As used herein a “probe” is defined as a nucleic acid, capable of binding to a target nucleic acid of complementary sequence through one or more types of chemical bonds, preferably through complementary base pairing via hydrogen bond formation. As used herein, a probe may include natural (i.e., A, G, U, C or T) or modified bases (7-deazaguanosine, inosine, locked nucleic acids, PNA's, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages. The design of appropriate oligonucleotide probes to specifically hybridize to a target nucleic acid is well within the level of skill in the art, based on the specification and the recited sequence information provided for the relevant genes. In one preferred embodiment, the oligonucleotide probes comprise at least 10 contiguous nucleotides of SEQ ID NO:1, 3, 5, 7, 9, 11, 13, 15, 17, 19, 21, or 23, depending on the gene to be assayed for expression product levels. In various further embodiments, the oligonucleotide probe may be at least 15, 20, 25, 30, 35, 40, 50, 75, 100, 250, 500, 1000, or more contiguous nucleotides of SEQ ID NO:1, 3, 5, 7, 9, 11, 13, 15, 17, 19, 21, or 23, depending on the gene to be assayed for expression product levels. Oligonucleotide probes may be used, for detection techniques including, but not limited to, in situ hybridization, branched DNA, sequencing, nuclease protection assay, or most preferably quantitative nuclease protection assay (qNPA), which may be array-based. In all embodiments, the oligonucleotide probes are optionally detectably labeled using standard methods in the art. Probes based on the sequences of the genes described herein may be prepared by any commonly available method. As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 95% sequence identity or more preferably about 100% sequence identity to said genes. In a most preferred embodiment, the oligonucleotide probes are fully complementary to the target mRNA expression product.

Nucleic acid hybridization in solution, on and array, or in situ simply involves contacting a probe and target nucleic acid under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. In a preferred embodiment a nuclease (e.g. S1) is added to destroy all oligonucleotides other than those that are hybridized together, and then the hybrids can be dissociated using (e.g.) base and heat, and the probe can subsequently be hybridized to an array and/or to other probes for its detection and quantitative measurement. Under low stringency conditions (e.g., low temperature and/or high salt) hybrid duplexes (e.g., DNA-DNA, RNA-RNA or RNA-DNA) will form even where the annealed sequences are not perfectly complementary. Thus specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch control, etc.).

In general, there is a tradeoff between hybridization specificity (stringency) and signal intensity. Thus, in a preferred embodiment, the wash is performed at the highest stringency that produces consistent results and that provides signal intensity greater than approximately two standard deviations of the average background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

In another aspect, the present invention provides oligonucleotide arrays which are useful for the practice of one or more of the methods of the invention. Isolated oligonucleotides for use in the oligonucleotide arrays are as described above for oligonucleotide probes, and preferably are from about 15 to about 150 nucleotides, more preferably from about 20 to about 100 in length. The oligonucleotide may be a naturally occurring oligonucleotide or a synthetic oligonucleotide. Oligonucleotides may be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art. Such arrays may contain an oligonucleotide which specifically hybridizes to one or more genes selected from the group consisting of GCET1, HLA-DQA1, HLA-DRB, HLA-DRA, ACTN1, COL3A1, PLAU, MYC, BCL6, LMO2, PDCD4, and SOD2. Preferably, such arrays may comprise a plurality of oligonucleotides which specifically hybridize to at least 2, 3, 4, 5, 6, 7, 8, 9, 10, 11, or 12 of the genes. In one preferred embodiment, the oligonucleotide arrays contain probes for no more than 16 distinct mRNAs. In various further embodiments, the oligonucleotide arrays contain probes for no more than 15, 14, 13, 12, 11, 10, 9, 8, 7, 6, 5, or 4 distinct mRNAs, which may include controls, as discussed above in the methods of the invention. Preferred embodiments disclosed herein for other aspects apply to this aspect as well unless the context clearly dictates otherwise, and may be combined with preferred embodiments described for this aspect. For example, the oligonucleotide arrays preferably comprise or consist of probes for the various preferred combinations of genes for use described above in the first and second aspects of the invention. In one preferred embodiment, oligonucleotide arrays preferably comprise or consist of probes for MYC and/or PDCD4. In another preferred embodiment, oligonucleotide arrays preferably comprise or consist of probes for 1, 2, 3, 4, 5, 6, 7, or 8 of HLA-DRB, HLA-DRA, HLA-DQA1, BCL6, ACTN1, COL3A1, LMO2, or PLAU. In various further preferred embodiments, oligonucleotide arrays preferably comprise or consist of probes for a combination of MYC and 1, 2, 3, 4, 5, or 6 of HLA-DRB, HLA-DRA, PLAU, BCL6, ACTN1, and LMO2; or a combination of PDCD4 and 1, 2, 3, 4, 5, or 6 of HLA-DRB, PLAU, BCL6, ACTN1, and LMO2. Each of these embodiments is particularly preferred for use in methods for prognosing an outcome of R-CHOP therapy on a DLBCL patient. In various further preferred embodiments, the oligonucleotide arrays may further comprise oligonucleotide probes for other genes listed in Tables 1-3 and 5. All of these embodiments can be combined with the preferred embodiments above in which oligonucleotide probes for no more than 16, 15, 14, 13, 12, 11, 10, 9, 8, 7, 6, 5, 4, 3, or 2 genes in total (including control genes) are present on the array, unless the context clearly dictates otherwise. Preferred methods may detect all or nearly all of the genes in the aforementioned tables. Any combination of genes may be employed, for example, a set of genes that are up-regulated and a set of genes that are down-regulated, as recited in the first and second aspects of the invention.

All arrays of the present invention may be formed on any suitable solid surface material. Examples of such solid surface materials include, but are not limited to, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots). When bound to a solid support, the oligonucleotide probes (or antibodies and/or aptamers) can be directly linked to the support, or attached to the surface via a linker. Thus, the solid support surface and/or the polynucleotide can be derivatized using methods known in the art to facilitate binding of the oligonucleotide probes (or antibodies and/or aptamers) to the solid support, so long as the derivitization does not eliminate detection of binding between the oligonucleotide probes (or antibodies and/or aptamers) and its target. Other molecules, such as reference or control nucleic acids, proteins, antibodies, and/or aptamers can be optionally immobilized on the solid surface as well. Methods for immobilizing such molecules on a variety of solid surfaces are well known to those of skill in the art.

Any hybridization assay format may be used, including solution-based and solid support-based assay formats. Solid supports containing oligonucleotide probes for differentially expressed genes of the invention can be filters, polyvinyl chloride dishes, silicon or glass based chips, etc. Such wafers and hybridization methods are widely available, for example, those disclosed by Beattie (WO 95/11755). Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, about 2, 10, 100, 1000 to 10,000; 100,000 or 400,000 of such features on a single solid support. The solid support, or the area within which the probes are attached may be on the order of a square centimeter. In addition to test probes that bind the target nucleic acid(s) of interest, the high density array can contain a number of control probes. The control probes fall into three categories referred to herein as (1) normalization controls; (2) expression level controls; and (3) mismatch controls. Normalization controls are oligonucleotide or other nucleic acid probes that are complementary to labeled reference oligonucleotides or other nucleic acid sequences that are added to the nucleic acid sample. Expression level controls are probes that hybridize specifically with constitutively expressed genes in the biological sample. Typical expression level control probes have sequences complementary to subsequences of constitutively expressed “housekeeping genes” (such as those in Table 5) invariant between samples with respect to treatment and varying only according to the number of cells in the sample, including, but not limited to the 13-actin gene, the PRKG1 gene, the TBP gene, transferrin receptor gene, the GAPDH gene, and the like. Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling. See, Pirrung et al., (1992) U.S. Pat. No. 5,143,854; Fodor et al., (1998) U.S. Pat. No. 5,800,992; Chee et al, (1998) U.S. Pat. No. 5,837,832 and Fodor et al. (WO 93/09668). Oligonucleotide probe arrays for expression monitoring can be made and used according to any techniques known in the art (see for example, Lockhart et al., (1996) Nat. Biotechnol. 14, 1675-1680; McGall et al., (1996) Proc. Nat. Acad. Sci. USA 93, 13555-13460). Such probe arrays may contain at least one or more oligonucleotides that are complementary to or hybridize to one or more of the genes described herein. Such arrays may also contain oligonucleotides that are complementary or hybridize to at least about 4, 5, 6, 7, 8, 9, 10, 15, 20, 30, 50, 70 or more the genes described herein. Quantitative nuclease protection arrays (qNPA), as those described in U.S. Pat. Nos. 6,232,066 and 6,238,869 are preferably employed, wherein probes used in such arrays comprise one or more of genes disclosed in Tables 1-3 or complements thereof. Methods for conducting qNPA assays in fixed tissue samples are described in PCT/US08/58837, which is incorporated herein by reference in its entirety.

In another preferred embodiment of the methods of all of the aspects and embodiments of the invention, detection or mRNA expression product level comprises the use of oligonucleotide primer pairs that are homologous to the mRNA to be detected, and which can be used in amplification assays, such as PCR, RT-PCR, RTQ-PCR, spPCR, and qPCR. The design of appropriate oligonucleotide primer pairs is well within the level of skill in the art, based on the specification and the recited sequence information provided for the relevant genes. As is well known in the art, oligonucleotide primers can be used in various assays (PCR, RT-PCR, RTQ-PCR, spPCR, qPCR, and allele-specific PCR, etc.) to amplify portions of a target to which the primers are complementary. Thus, a primer pair would include both a “forward” and a “reverse” primer, one complementary to the sense strand (ie: the strand shown in the sequences provided herein) and one complementary to an “antisense” strand (ie: a strand complementary to the strand shown in the sequences provided herein), and designed to hybridize to the target so as to be capable of generating a detectable amplification product from the target of interest when subjected to amplification conditions. The sequences of each of the target nucleic acids are provided herein, and thus, based on the teachings of the present specification, those of skill in the art can design appropriate primer pairs complementary to the target of interest (or complements thereof). In various preferred embodiments, each member of the primer pair is a single stranded DNA polynucleotide at least 15, 16, 17, 18, 19, 20, 21, 22, 23, 24, 25, 26, 27, 28, 29, 30, 31, 35, 40, 45, 50, or more nucleotides in length that are fully complementary to the expression product target. In one preferred embodiment, each member of an oligonucleotide primer pair comprises at least 10 contiguous nucleotides of SEQ ID NO:1, 3, 5, 7, 9, 11, 13, 15, 17, 19, 21, or 23, depending on the gene to be assayed for expression product levels. In various further embodiments, the each member of an oligonucleotide primer pair comprises at least 15, 20, 25, 30, 35, 40, 50, 75, 100, or more contiguous nucleotides of SEQ ID NO:1, 3, 5, 7, 9, 11, 13, 15, 17, 19, 21, or 23, depending on the gene to be assayed for expression product levels. In a most preferred embodiment, the primer pairs are fully complementary over their entire length to the target expression product. In all embodiments, the oligonucleotide primers are optionally detectably labeled using standard methods in the art. PCR, RT-PCR, and other amplification techniques, including quantitative amplification techniques, can be carried out using methods well known to those of skill in the art based on the teachings herein.

In another preferred embodiment of the methods of all of the aspects and embodiments of the invention, detection or protein expression product level comprises the use of antibody or aptamer probes that selectively bind to the protein to be detected. The design of appropriate antibodies and aptamers is well within the level of skill in the art, based on the specification and the recited sequence information provided for the relevant genes, and the knowledge of those of skill in the art in aptamer design. In one preferred embodiment, the antibodies or aptamers selectively bind to a protein of SEQ ID NO:2, 4, 6, 8, 10, 12, 14, 16, 18, 20, 22, or 24, depending on the gene to be assayed for expression product levels. Antibodies may be used, for detection techniques including, but not limited to, in immunoblotting, ELISA, ligand binding assays, and protein array analysis.

In another aspect, the present invention provides antibody micro-arrays comprising or consisting of one or more antibodies and/or aptamers (nucleic acids or peptides that bind a specific target molecule.) that selectively bind to a protein of SEQ ID NO:2, 4, 6, 8, 10, 12, 14, 16, 18, 20, 22, or 24. The term “antibody,” as used herein, is intended to include whole antibodies, for example, of any isotype (IgG, IgA, IgM, IgE, etc.), and includes fragments thereof which are also specifically reactive with a vertebrate (e.g., mammalian) protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies. Preferably, such arrays may comprise a plurality of antibodies and/or aptamers which selectively bind to at least 2, 3, 4, 5, 6, 7, 8, 9, 10, 11, or 12 of the recited protein expression products. In one preferred embodiment, the antibody and/or aptamer arrays contain probes for no more than 16 distinct proteins. In various further embodiments, the antibody and/or aptamer arrays contain probes for no more than 15, 14, 13, 12, 11, 10, 9, 8, 7, 6, 5, or 4 distinct proteins, which may include controls, as discussed above in the methods of the invention. Preferred embodiments disclosed herein for other aspects apply to this aspect as well unless the context clearly dictates otherwise, and may be combined with preferred embodiments described for this aspect. For example, the antibody and/or aptamer arrays preferably comprise or consist of probes for the various preferred combinations of genes for use described above in the first and second aspects of the invention. In one preferred embodiment, antibody and/or aptamer arrays preferably comprise or consist of probes for MYC and/or PDCD4, In another preferred embodiment, antibody and/or aptamer arrays preferably comprise or consist of probes for 1, 2, 3, 4, 5, 6, 7, or 8 of HLA-DRB, HLA-DRA, HLA-DQA1, BCL6, ACTN1, COL3A1, LMO2, or PLAU. In various further preferred embodiments, antibody and/or aptamer arrays preferably comprise or consist of probes for a combination of MYC and 1, 2, 3, 4, 5, or 6 of HLA-DRB, HLA-DRA, PLAU, BCL6, ACTN1, and LMO2; or a combination of PDCD4 and 1, 2, 3, 4, 5, or 6 of HLA-DRB, PLAU, BCL6, ACTN1, and LMO2. Each of these embodiments is particularly preferred for use in methods for prognosing an outcome of R-CHOP therapy on a DLBCL patient. In various further preferred embodiments, the antibody and/or aptamer arrays may further comprise antibody probes for other genes listed in Tables 1-3 and 5. All of these embodiments can be combined with the preferred embodiments above in which antibodies and/or aptamers for no more than 16, 15, 14, 13, 12, 11, 10, 9, 8, 7, 6, 5, 4, 3, or 2 genes in total (including control genes) are present on the array, unless the context clearly dictates otherwise. Antibody and/or aptamer molecules may comprise detectable labels; methods for labeling such molecules are known in the art.

The invention further comprises kits useful for the practice of one or more of the methods of the invention, wherein the kits comprise one or more of the compositions of the invention (e.g., oligonucleotide probes, oligonucleotide probe arrays, oligonucleotide primer pairs, antibodies, aptamers, antibody arrays, aptamer arrays) and instructions for its use in prognosing a treatment outcome for DLBCL patients. The invention further relates to “kits” combining, in different combinations, high-density oligonucleotide, antibody, and/or aptamer arrays, reagents for use with the arrays, signal detection and array-processing instruments, gene expression databases and analysis, manuals and database management software described above. The databases packaged with the kits are a compilation of expression patterns from human or laboratory animal genes and gene fragments (corresponding to the genes of Tables 1-3 and Table 5). Data is collected from a repository of both normal and diseased animal tissues and provides reproducible, quantitative results, i.e., the degree to which a gene is up-regulated or down-regulated under a given condition. In some preferred embodiments, a kit may contain one or more oligonucleotides arrays as described above. The solid support may be a high-density oligonucleotide array. Kits may further comprise one or more reagents for use with the arrays, one or more signal detection and/or array-processing instruments, one or more gene expression databases and one or more analysis and database management software packages. Examples of such kit uses include kits for in situ hybridization, for PCR, for bDNA, for the NanoString technology, and for sequencing.

The present invention includes relational databases containing sequence information, for instance for the genes for analysis in the present invention, as well as gene expression information in various cell or tissue samples, and patient treatment and response or outcome information or other diagnostic information (such as determination of disease stage, e.g. DLBCL) or patient risk assessment (by e.g. IPI score). Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequences database and a gene expression database. Methods for the configuration and construction of such databases are widely available, for instance, see Akerblom et al., (1999) U.S. Pat. No. 5,953,727, which is herein incorporated by reference in its entirety. The databases of the invention may be linked to an outside or external database. In a preferred embodiment, the external database is GenBank and the associated databases maintained by the National Center for Biotechnology Information (NCBI). The databases of the invention may be used to produce, among other things, electronic Northern blots to allow the user to determine the cell type or tissue in which a given gene is expressed and to allow determination of the abundance or expression level of a given gene in a particular tissue or cell. The databases of the invention may also be used to present information identifying the expression level in a tissue or cell of a set of genes comprising at least two genes selected from the group consisting of GCET1, HLA-DQA1, HLA-DRB, HLA-DRA, ACTN1, COL3A1, PLAU, MYC, BCL6, LMO2, PDCD4, and SOD2 comprising the step of comparing the expression product level of at the least two genes in the tissue to the level of expression of the gene in the database. Such methods may be used to predict the physiological state of a given tissue by comparing the expression product level of the two or more genes from a sample to the expression levels found in a normal tissue, a cancerous tissue, or a malignant tumor or the tissue of patients with the same disease (e.g. DLBCL) and treatment (e.g. R-CHOP) or other patients with a different clinical outcome. Such methods may also be used in the drug or agent screening assays as described herein. Databases and software designed for use with use with microarrays is discussed in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences. Any appropriate computer platform may be used to perform the necessary comparisons between sequence information, gene expression information and any other information in the database or provided as an input. For example, a large number of computer workstations are available from a variety of manufacturers, such as those available from Silicon Graphics. Client-server environments, database servers and networks are also widely available and appropriate platforms for the databases of the invention.

Fixed Tissue Samples

Methods for conducting qNPA assays in fixed (or insoluble) specimen are described in PCT/US08/58837, which is incorporated herein by reference in its entirety. The accurate measurement of genes, and in particular gene expression from fixed tissue has many benefits. In the case of clinical samples the described process permits target oligonucleotides to be measured without necessitating a change in clinical practice—directly from fixed tissue without having to prepare frozen samples. There are vast stores of archived fixed material that could be used for retrospective studies to identify and validate biomarkers and target genes, or for development and validation of a monitoring, prognostic, or diagnostic assay, or for the association of safety with gene expression, or for the understanding of disease processes, etc. The present invention solves the limitations of analyzing gene expression in fixed samples. For example, it is known that measurement by PCR or hybridization methods requires large amounts of tissue and involves complex extraction and sample preparation methods. In addition, it is often observed that the quality of measurement decreases as a function of how long the tissue has been stored. In contrast, in situ measurements (where the RNA or protein is labeled and visualized in the tissue) can be performed on freshly fixed tissue or archived tissue and produce similar quality data. The present invention therefore provides methods for detecting expression product levels from fixed tissues comprising recovering a probe from the tissue wherein said probe serves as the basis for measurement, rather than the native oligonucleotide itself. The instant invention is further drawn to the use of nuclease protection as a method to measure oligonucleotides from fixed tissue. The method disclosed by the instant invention therefore permits the measurement of cross-linked oligonucleotides as well as soluble oligonucleotides. The measurement of a biological target in fixed or preserved samples is a technically challenging venture. Proteins are known to denature, often losing antigenicity (i.e., antibody recognition) in the process. Carbohydrates can be chemically altered, particularly those associated with peptides and proteins in a glycoprotein moiety. Nucleic acids can undergo cross-linking between one another, and other molecules, including proteins, lipids, and carbohydrates, in the cellular milieu. The recovery and analysis of these molecules is an expensive and a time-consuming process. Measurements from fixed samples can be made using a single array, both low and high density, and both fixed (capture probes printed as the array) or programmable (combinations of printed anchors and added programming/capture linkers), or multiple arrays such as might be printed in the wells of a microplate or on bead arrays, including beads in solution measuring multiple genes in each sample, or by the tagging of the nuclease protection proteins with or without fixation to a surface and imaging, or by use of gels, electrophoresis, chromatography, mass spectroscopy, sequencing, as mixtures, or as individual targets detected in each reaction mixture, such as in a conventional microplate assay, or by PCR (or other amplification method) of the nuclease protection probe or by hybrid capture, or other method one skilled in the art might use. The measurement of different forms of oligonucleotide from fixed samples, both single samples as well as to make comparisons between samples, including for instance, diseased versus normal, treated versus control, or any combinations thereof, can be performed.

The measurement of protein using aptamers, or other probes, are also permissible with the instant invention. The instant invention also relates to measurement of proteins and oligonucleotides simultaneously using appropriate probes. In yet another aspect, the instant invention relates to the hybridization (or binding) of probes to cross-linked (and soluble) RNA, and then removal and measurement of the probe, or probe/target molecule, even where the target molecule may be damaged, fractured or cleaved, but the probe or probe complex is intact or held together sufficiently. Any method where the probe associates with both cross-linked or surface bound target molecule (e.g. oligonucleotide or e.g. RNA) and soluble target molecule, or associated only with the cross-linked or surface bound target molecule, is reduced to an analyzable amount relative to the target molecule, then removed from the tissue and measured.

The invention provides a method for detecting at least one insoluble target, which comprises contacting a sample which may comprise the target(s) with a combination as described above, under conditions effective for said target(s) to bind to said combination. Another embodiment is a method for determining an RNA expression pattern, which comprises incubating a sample which comprises as target(s) at least two RNA molecules with a combination as described above, wherein at least one probe of the combination is a nucleic acid (e.g., oligonucleotide) which is specific (i.e. selective) for at least one of the insoluble RNA targets, under conditions which are effective for specific hybridization of the RNA target(s) to the probe(s). Another embodiment is a method for identifying an agent (or condition(s)) that modulates an RNA expression pattern, which is the method described above for determining an RNA expression pattern, further comprising comparing the RNA expression pattern produced in the presence of said agent (or condition(s)) to the RNA expression pattern produced under a different set of conditions. Compositions and agents that modulate gene or RNA expression pattern, for example, CHOP therapy (with or without anti-CD20 antibody immunotherapy) have been described in the aforementioned paragraphs.

### DEFINITIONS

As used herein, the term “nucleic acid” refers to polynucleotides such as deoxyribonucleic acid (DNA) and, where appropriate, ribonucleic acid (RNA). The term should also be understood to include, as equivalents, analogs of either RNA or DNA made from nucleotide analogs and, as applicable to the embodiment being described, single-stranded (sense or antisense) and double-stranded polynucleotides. Chromosomes, cDNAs, mRNAs, rRNAs, and ESTs are representative examples of molecules that may be referred to as nucleic acids.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens), and the like. The term “fluorescer” refers to a substance or a portion thereof which is capable of exhibiting fluorescence in the detectable range. Particular examples of labels which may be used in the present invention include fluorescein, rhodamine, dansyl, umbelliferone, Texas red, luminol, NADPH, alpha-beta—galactosidase, and horseradish peroxidase.

The term “protein” is used interchangeably herein with the terms “peptide” and “polypeptide.”

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

The invention will be explained below with reference to the following non-limiting examples.

### Example 1

### Patient Materials

Three 5-micron unstained cuts from FFPET blocks were used from 93 cases of DLBCL treated primarily with cyclophosphamide, hydroxydaunorubicin, oncovorin (vincristine) and prednisone (CHOP) or similar CHOP-like chemotherapy and 116 cases treated with Rituximab plus CHOP. Cases of transformed lymphomas were excluded. Frozen blocks from the CHOP-alone cases had been analyzed as part of a prior publication. 1 As previously reported, these cases had undergone consensus review by a panel of expert hematopathologists and confirmed as DLBCL. The R-CHOP cases were taken from the current case files at the University of Arizona British Columbia Cancer Agency, and Oregon Health Sciences Center. Of these 116 R-CHOP cases, frozen blocks from 32 were also used in another study and had undergone review by an expert panel (Lenz et al., Blood, 2007). All tissues used for this retrospective study came from pre-treatment diagnostic biopsies using excess diagnostic tissue under IRB approved protocols.

Assay Methods:

The performance of the ArrayPlate™ assay customized for use in DLBCL has been described previously by our group (Roberts et al., 2007). Three 5 micron unstained tissue sections were lysed, denatured, and permeabilized by heating in HTG Lysis Buffer. The samples were then frozen and sent for analysis. 50-mer probes specific for the genes of interest were incubated with the samples, forming specific probe-mRNA duplexes, then unhybridized probes were digested by S1 Nuclease. Next, alkaline hydrolysis destroyed the mRNA in the duplexes, leaving intact probes with stoichiometric concentrations proportional to the amounts of specific mRNA originally present. After neutralization, samples were transferred to ArrayPlates™ for probe detection. The ArrayPlates™ contained a universal array of 16 unique, covalently-bound, 25-mer “anchor” oligonucleotides spotted in a 4×4 grid on the bottom of each well. This universal array was modified to bind 50-mer probes for the genes of interest at pre-selected positions by exposing the array to a mixture of 50-mer Programming Linker oligonucleotides that contained a 25-mer sequence to bind one of the probes at one end, and a 25-mer sequence to bind one of the anchor oligonucleotides on the other end. Three different mixtures of Programming Linker oligonucleotides distributed across 3 ArrayPlate™ wells were required to measure all the genes of interest in our assay.

After hybridization, probes from the sample were bound to array elements by the Programming Linker oligonucleotides. A mixture of Detection Linker oligonucleotides was added. The 50-mer Detection Linkers contained a 25-mer sequence that bound sample probe on the end not bound by the Programming Linker probe on one end, and a common 25-mer sequence to bind a Detection Probe on the other. Detection Probe was added, which bound to all the Detection Linkers. The Detection Probe contained bound horseradish peroxidase. Upon the addition of chemiluminescent peroxidase substrate (Lumigen PS-atto, Lumigen, Inc., Southfield, Mich.) each array element gave off light proportional to the amount of sample probe bound at that position.

The signals for all 1,536 elements in an ArrayPlate™ were recorded simultaneously by imaging the plate from the bottom with a CCD-based Omix Imager (HTG). Images were analyzed using Vuescript software (HTG) which calculated average pixel intensity for each element to determine expression levels for each gene. Expression levels were normalized to the housekeeping gene TBP.

As previously, we used the key genes identified as prognostically important in 4 previous papers in DLBCL which accounted for 36 genes of interest. Because of the heterogeneity of cellular composition in human tumor samples, we also included probes designed to test the tumor composition for B-cells (CD19, CD20), T-cells (CD3) and histiocytes (CD68). Two housekeeping genes, TBP and PRKG1, were chosen based previously published work assessing the utility of different endogenously expressed genes as housekeeping genes, which identified these 2 genes as stably expressed at moderate or low levels in different types of lymphomas by qRT-PCR. These 2 housekeeping genes were repeated at diagonal corners in each of the 3 wells used to create the assay. An oligo dT probe was added in order to assess the quantity of mRNA in the sample (since an oligo dT probe should detect all mRNA which has a poly-A tail). However, for technical reasons due to the stringency of the assay, this probe was non-functional and not further utilized. A probe for cytochrome oxidase was also initially included because it is coded in mitochondrial DNA, and should be expressed at high levels. This turned out to bind both DNA and RNA, and so gave an extremely bright and generally oversaturated signal and was therefore not further considered, except that it could be used to distinguish whether there was insufficient material for the assay, or whether, if it had disappeared entirely, the sample was too degraded for use.

For each of the 44 genes of interest, four specific probes were designed though not all were synthesized. ArrayBuilder™ 2.0 software (HTG) was used to design the oligonucleotides required for the assay to measure target transcripts in groups of 16. Briefly, with the user providing the accession numbers for the target genes and assigning their position in the array, the software retrieved each mRNA sequence from GenBank and ranked successive 50-mer stretches of the target gene sequences according to the melting temperature (Tm) of their 5′- and 3′-constituent 25mers, giving preference to those 50-mers for which the Tm of each of the two 25-mer halves was nearest to 68° C. The four highest ranked and non-overlapping 50-mer sequences for each of the 16 target mRNA species were subjected to BLAST to identify homologous sequences. Sequences with homology to other genes were rejected and replaced with the next highest-ranking 50-mer sequence that was in turn submitted to BLAST. Sequences without significant homology were retained. The software then created output files containing the sequences of the four oligonucleotides (Programming Linker, Protection Probe, Detection Linker and Attenuation Fragment) required to measure a given 50-mer target in the assay.

Table 2 lists the names of the genes of interest, position at which probes begin for that gene, and the sequence of the target, wherein the designed probes are reverse complementary to the recited sequence.

The key genes identified as prognostically important in four previous papers of DLBCL, which accounted for 36 genes of interest were used in this study (Rosenwald et al., N Engl J. Med. 2002; 346:1937-1947; Tome et al., Blood. 2005; 106:3594-3601; Shipp et al., Nat Med. 2002; 8:68-74; Lossos et al., N Engl J. Med. 2004; 350:1828-1837). The genes are listed in TABLE 1 in the order in which they were listed in the original references. The housekeeping gene, TATA Box Binding Protein (TBP) was chosen for normalizing the data based on its stable expression at moderate levels in 12 lymphoma cell lines and 80 B and T cell lymphoma samples as compared to 11 other “housekeeping” genes using q-RT-PCR (Lossos et al., Leukemia. 2003; 17:789-795) as well as previous experience with this gene in the ArrayPlate assay showing it to be moderately expressed with minimal variability in all samples tested to date (Roberts et al., Laboratory Investigation. 2007; 87:979-997).

TABLE 1
List of prognostic genes identified in prior studies
of CHOP treated patients assessed using ArrayPlate.

| Accession # | Original ref | ArrayPlate name | Reference* |
|---|---|---|---|
| NM_138931 | bcl-6 (SEQ ID NOs: 1, 2) | BCL6 | Rosenwald 1 |
| | | | Lossos 6 |
| NM_175739 | IMAGE 1334260 (SEQ ID NOs: 3, 4) | GCET1 | Rosenwald 2 |
| | | (SERPINA9) | |
| NM_152785 | IMAGE 814622 (SEQ ID NOs: 25, 26) | GCET2 | Rosenwald 3 |
| NM_033554 | HLA-DPα (SEQ ID NOs: 27, 28) | HLA-DPA1 | Rosenwald 4 |
| NM_002122 | HLA-DQα (SEQ ID NOs: 9, 10) | HLA-DQA1 | Rosenwald 5 |
| NM_019111 | HLA-DRα (SEQ ID NOs: 11, 12) | HLA-DRA | Rosenwald 6 |
| NM_002124 | HLA-DRβ (SEQ ID NOs: 13, 14) | HLA-DRB | Rosenwald 7 |
| NM_001102 | α-actinin (SEQ ID NOs: 15, 16) | ACTN1 | Rosenwald 8 |
| NM_000090 | collagen type III α 1 (SEQ ID NOs: 17, 18) | COL3A1 | Rosenwald 9 |
| NM_001901 | connective-tissue growth factor | CTGF | Rosenwald 10 |
| | (SEQ ID NOs: 35, 36) | | |
| NM_212482 | Fibronectin (SEQ ID NO: 47, 48) | FN1 | Rosenwald 11; |
| | | | Lossos 5 |
| NM_014745 | KIAA0233 (SEQ ID NOs: 73, 74) | FAM38A | Rosenwald 12 |
| NM_002658 | urokinase plasminogen activator | PLAU | Rosenwald 13 |
| | (SEQ ID NOs: 5, 6) | | |
| NM_002467 | MYC (SEQ ID NOs: 7, 8) | MYC | Rosenwald 14 |
| NM_019095 | E21G3 (Nucleostemin) (SEQ ID | C20orf155 | Rosenwald 15 |
| | NOs: 29, 30) | | |
| NM_006993 | NPM3 (SEQ ID NOs: 31, 32) | NPM3 | Rosenwald 16 |
| NM_001718 | BMP6 (SEQ ID NOs: 33, 34) | BMP6 | Rosenwald 17 |
| NM_005574 | LM02 (SEQ ID NOs: 19, 20) | LMO2 | Lossos 1 |
| NM_000633 | BCL2 (SEQ ID NOs: 37, 38) | BCL2 | Lossos 2 |
| NM_002983.1 | SCYA3 (SEQ ID NOs: 39, 40) | CCL3 | Lossos 3 |
| NM_001759.2 | CCND2 (SEQ ID NOs: 41, 42) | CCND2 | Lossos 4 |
| NM_001939 | dystrophin related protein 2 (SEQ | DRP2 | Shipp 1 |
| | ID NOs: 43, 44) | | |
| NM_002738 | PRKACB protein kinase C-beta-1 | PRKCB1 | Shipp 2 |
| | (SEQ ID NOs: 45, 46) | | |
| NM_014456 | H731 nuclear antigen (SEQ ID NOs: 21, 22) | PDCD4 | Shipp 3 |
| NM_005909 | 3′ UTR of unknown protein (SEQ | MAP1B | Shipp 4 |
| | ID NOs: 49, 50) | | |
| NM_005077 | Transducin-like enhancer protein 1 | TLE1 | Shipp 5 |
| | (SEQ ID NOs: 51, 52) | | |
| NM_014251 | Uncharacterized (SEQ ID NOs: 53, 54) | SLC25A13 | Shipp 6 |
| NM_002600 | Phosphodiesterase 4B, cAMP | PDE4B | Shipp 7 |
| | specific (SEQ ID NOs: 55, 56) | | |
| NM_001497 | beta 1 ,4-galactosyltransferase, | B4GALT1 | Shipp 8 |
| | polypeptide 1 (SEQ ID NOs: 57, 58) | | |
| NM_002739 | PRKCG Protein kinase C, gamma | PRKCG | Shipp 9 |
| | (SEQ ID NOs: 59, 60) | | |
| NM_002557 | Oviductal glycoprotein (SEQ ID | OVGP1 | Shipp 10 |
| | NOs: 61, 62) | | |
| NM_173198 | Mitogen induced nuclear orphan | NR4A3 | Shipp 11 |
| | receptor (MINOR) (SEQ ID NOs: | | |
| | 63, 64) | | |
| NM_012256 | Zinc-finger protein C2H2-150 | ZNF212 | Shipp 12 |
| | (SEQ ID NOs: 65, 66) | | |
| NM_000867 | 5-Hydroxytryptamine 2B receptor | HTR2B | Shipp 13 |
| | (SEQ ID NOs: 69, 70) | | |
| NM_001752 | Catalase (SEQ ID NOs: 71, 72) | CAT | Tome 1 |
| NM_000636 | Manganese superoxide dismutase | SOD2 | Tome 2 |
| | (SEQ ID NOs: 23, 24) | | |
| M34960 | TATA Box binding protein | TBP** | Lossos |
| | (SEQ ID NOs: 67, 68) | | |

Legend:
*Papers represented in this table include:
(a) Rosenwald A, et al. N Engl J Med, 2002; 346: 1937-1947;
(b) Shipp M A, et al, Nat Med. 2002; 8: 68-74;
(c) Losses I S, et al, N Engl J Med. 2004; 350: 1828-1837;
(d) Tome M E et al Blood. 2005; 106: 3594-3601;
**Lossos I S, et al Leukemia. 2003; 17: 789-795.

TABLE 2

| | Name in original | Gene | |
|---|---|---|---|
| Accession # | reference | Position | Target Sequence (5′ Start) |
| NM_006258 | PRKG1 | 465 | CGGTGGAGTATGGCAAGGACAGTTGCATCATCAAAG |
| | | | AAGGAGACGTGGGG |
| | | | (SEQ ID NO: 75) |
| NM_175739 | IMAGE 1334260 | 934 | TGCACCAGAAAGAGCAGTTCGCTTTTGGGGTGGATA |
| | | | CAGAGCTGAACTGC |
| | | | (SEQ ID NO: 76) |
| NM_152785 | IMAGE 814622 | 222 | GCAAAGCCCCAAACAGAGAACATCCAGATGCTGGGA |
| | | | TCACCATATCGCTG |
| | | | (SEQ ID NO: 77) |
| NM_033554 | HLA-DPα | 236 | AAGAAGGAGACCGTCTGGCATCTGGAGGAGTTTGGC |
| | | | CAAGCCTTTTCCTT |
| | | | (SEQ ID NO: 78) |
| NM_002122 | HLA-DQα | 1391 | GCAACAATGAAATTAATGGATACCGTCTGCCCTTGGC |
| | | | CCAGAATTGTTAT |
| | | | (SEQ ID NO: 79) |
| NM_019111 | HLA-DRα | 335 | TGGCCAACATAGCTGTGGACAAAGCCAACCTGGAAA |
| | | | TCATGACAAAGCGC |
| | | | (SEQ ID NO: 80) |
| NM_002124 | HLA-DRβ | 14 | TGGAAACAGTTCCTCGGAGTGGAGAGGTTTACACCT |
| | | | GCCAAGTGGAGCAC |
| | | | (SEQ ID NO: 81) |
| NM_001102 | α-actinin | 1922 | AGACCTACCACGTCAATATGGCGGGCACCAACCCCT |
| | | | ACACAACCATCACG |
| | | | (SEQ ID NO: 82) |
| NM_000090 | collagen type III α 1 | 4349 | CAGTTCTGGAGGATGGTTGCACGAAACACACTGGGG |
| | | | AATGGAGCAAAACA |
| | | | (SEQ ID NO: 83) |
| NM_001901 | connective-tissue growth | 1698 | TTCAGGAATCGGAATCCTGTCGATTAGACTGGACAG |
| | factor | | CTTGTGGCAAGTGA |
| | | | (SEQ ID NO: 84) |
| NM_212482 | fibronectin | 7340 | GGGAGAAAATGGCCAGATGATGAGCTGCACATGTCT |
| | | | TGGGAACGGAAAAG |
| | | | (SEQ ID NO: 85) |
| NM_014745 | KIAA0233 | 3947 | GTGCTATGGCCTCTGGGACCATGAGGAGGACTCACC |
| | | | ATCCAAGGAGCATG |
| | | | (SEQ ID NO: 86) |
| NM_002658 | urokinase plasminogen | 835 | GGGTCGCTCAAGGCTTAACTCCAACACGCAAGGGGA |
| | activator | | GATGAAGTTTGAGG |
| | | | (SEQ ID NO: 87) |
| NM_002467 | c-myc | 1477 | CCACACATCAGCACAACTACGCAGCGCCTCCCTCCAC |
| | | | TCGGAAGGACTAT |
| | | | (SEQ ID NO: 88) |
| NM_138931 | bcl-6 | 1948 | GATTCTAGCTGTGAGAACGGGGCCTTCTTCTGCAATG |
| | | | AGTGTGACTGCCG |
| | | | (SEQ ID NO: 89) |
| M34960 | TATA Box binding protein | 562 | CGAAACGCCGAATATAATCCCAAGCGGTTTGCTGCG |
| | | | GTAATCATGAGGAT |
| | | | (SEQ ID NO: 90) |
| M34960 | TATA Box binding protein | 461 | CAGCTTCGGAGAGTTCTGGGATTGTACCGCAGCTGCA |
| | | | AAATATTGTATCC |
| | | | (SEQ ID NO: 91) |
| M34960 | TATA Box binding protein | 774 | GGTGGGGAGCTGTGATGTGAAGTTTCCTATAAGGTTA |
| | | | GAAGGCCTTGTGC |
| | | | (SEQ ID NO: 92) |
| NM_006258 | PRKG1 | 465 | CGGTGGAGTATGGCAAGGACAGTTGCATCATCAAAG |
| | | | AAGGAGACGTGGGG |
| | | | (SEQ ID NO: 93) |
| NM_006993 | NPM3 | 418 | GGCACCAGATTGTTACGATGAGCAATGATGTTTCTGA |
| | | | GGAGGAGAGCGAG |
| | | | (SEQ ID NO: 94) |
| NM_001718 | BMP6 | 1566 | ACCTTGGTTCACCTTATGAACCCCGAGTATGTCCCCA |
| | | | AACCGTGCTGTGC |
| | | | (SEQ ID NO: 95) |
| NM_001718 | BMP6 | 1807 | GGTGGGACGATGAGACTTTGAAACTATCTCATGCCA |
| | | | GTGCCTTATTACCC |
| | | | (SEQ ID NO: 96) |
| NM_001718 | BMP6 | 1031 | GCACAGAGACTCTGACCTGTTTTTGTTGGACACCCGT |
| | | | GTAGTATGGGCCT |
| | | | (SEQ ID NO: 97) |
| NM_001718 | BMP6 | 2458 | GCTCACCTCTTCTTTACCAGAACGGTTCTTTGACCAG |
| | | | CACATTAACTTCT |
| | | | (SEQ ID NO: 98) |
| NM_005574 | LM02 | 2012 | AAGGCCTTAAGCTTTGGACCCAAGGGAAAACTGCAT |
| | | | GGAGACGCATTTCG |
| | | | (SEQ ID NO: 99) |
| NM_000633 | BCL2 | 2165 | CCTGCTTTTAGGAGACCGAAGTCCGCAGAACCTGCCT |
| | | | GTGTCCCAGCTTG |
| | | | (SEQ ID NO: 100) |
| NM_002983. | SCYA3 | 715 | ATGCTTTTGTTCAGGGCTGTGATCGGCCTGGGGAAAT |
| 1 | | | AATAAAGCACGCT |
| | | | (SEQ ID NO: 101) |
| NM_002983. | SCYA3 | 30 | CCTTTCTTGGCTCTGCTGACACTCGAGCCCACATTCC |
| 1 | | | GTCACCTGCTCAG |
| | | | (SEQ ID NO: 102) |
| NM_002983. | SCYA3 | 127 | TGGCTCTCTGCAACCAGTTCTCTGCATCACTTGCTGC |
| 1 | | | TGACACGCCGACC |
| | | | (SEQ ID NO: 103) |
| NM_002983. | SCYA3 | 571 | GTGTGTTTGTGATTGTTTGCTCTGAGAGTTCCCCTGTC |
| 1 | | | CCCTCCCCCTTC |
| | | | (SEQ ID NO: 104) |
| NM_001759. | CCND2 | 3666 | GCGAGTAGATGAACCTGCAGCAAGCAGCGTTTATGG |
| 2 | | | TGCTTCCTTCTCCC |
| | | | (SEQ ID NO: 105) |
| NM_001939 | DRP2 dystrophin related | 871 | AGCAAAGATACCTCCCCGAAACAGCGGATCCAGAAT |
| | protein 2 | | CTCAGCCGCTTTGT |
| | | | (SEQ ID NO: 106) |
| NM_001939 | DRP2 dystrophin related | 3282 | CACTGGCCCCACATTCCTCAACTAGTATTATTTGGGC |
| | protein 2 | | TCTGGGCAGCAGC |
| | | | (SEQ ID NO: 107) |
| NM_001939 | DRP2 dystrophin related | 1030 | GGGGCAATGGAGGAACTAAGCACTACTCTAAGCCAA |
| | protein 2 | | GCTGAGGGAGTCCG |
| | | | (SEQ ID NO: 108) |
| NM_001939 | DRP2 dystrophin related | 3038 | GACAGACCACTCCAGATACCGAGGCTGCAGATGATG |
| | protein 2 | | TGGGGTCAAAGAGC |
| | | | (SEQ ID NO: 109) |
| NM_002738 | PRKACB protein kinase C- | 2787 | AAAAGCACTTCAAGGGGTCAAAGGGCAACCAGCTTG |
| | beta-1 | | GGTGCTACCTCAGT |
| | | | (SEQ ID NO: 110) |
| NM_014456 | H731 nuclear antigen | 518 | CAACCAGTCCAAAGGGAAGGTTGCTGGATAGGCGAT |
| | | | CCAGATCTGGGAAA |
| | | | (SEQ ID NO: 111) |
| NM_005909 | 3′ UTR of unknown protein | 7037 | CAAAACCAGCGGGCTTGAAAGAATCCTCGGATAAAG |
| | | | TGTCCAGGGTGGCT |
| | | | (SEQ ID NO: 112) |
| NM_005077 | Transducin-like enhancer | 3039 | TTCTTTCTGGGTGATCTGGGGATCACGCCTTGCCCAA |
| | protein 1 | | GTGTGAGATTACC |
| | | | (SEQ ID NO: 113) |
| NM_005077 | Transducin-like enhancer | 1703 | TTGATCCTCCCCCTCACATGAGAGTACCTACCATTCC |
| | protein 1 | | TCCAAACCTGGCA |
| | | | (SEQ ID NO: 114) |
| NM_005077 | Transducin-like enhancer | 1312 | GCCTCCTCGGCAAGTTCCACTTCTTTGAAATCCAAAG |
| | protein 1 | | AAATGAGCTTGCA |
| | | | (SEQ ID NO: 115) |
| NM_005077 | Transducin-like enhancer | 1255 | GGAATCGACAAAAATCGCCTGCTAAAGAAGGATGCT |
| | protein 1 | | TCTAGCAGTCCAGC |
| | | | (SEQ ID NO: 116) |
| NM_014251 | Uncharacterized | 1662 | GCTTCCTTTGCAAATGAAGATGGGCAGGTTAGCCCA |
| | | | GGAAGCCTGCTCTT |
| | | | (SEQ ID NO: 117) |
| NM_014251 | Uncharacterized | 2037 | CCTGATCACGTTGGGGGCTACAAACTGGCAGTTGCTA |
| | | | CATTTGCAGGGAT |
| | | | (SEQ ID NO: 118) |
| NM_014251 | Uncharacterized | 890 | GGAGGAGTTTGTTCTGGCAGCTCAGAAATTTGGTCAG |
| | | | GTTACACCCATGG |
| | | | (SEQ ID NO: 119) |
| NM_014251 | Uncharacterized | 1536 | CGAGTCAGTGCTCTGTCTGTCGTGCGGGACCTGGGGT |
| | | | TTTTTGGGATCTA |
| | | | (SEQ ID NO: 120) |
| NM_002600 | PDE4B Phosphodiesterase | 2128 | CACCACCACTGGACGAGCAGAACAGGGACTGCCAGG |
| | 4B, cAMP-specific | | GTCTGATGGAGAAG |
| | | | (SEQ ID NO: 121) |
| NM_019095 | E21G3 (Nucleostemin) | 474 | GCTCGAAACTGGGCCAATCAAAGATCAGCTTTGGGA |
| | | | AGTGCTCTTGATCC |
| | | | (SEQ ID NO: 122) |
| M34960 | TATA Box binding protein | 537 | CCTAAAGACCATTGCACTTCGTCGCCGAAACGCCGA |
| | | | ATATAATCCCAAGC |
| | | | (SEQ ID NO: 123) |
| M34960 | TATA Box binding protein | 461 | CAGCTTCGGAGAGTTCTGGGATTGTACCGCAGCTGCA |
| | | | AAATATTGTATCC |
| | | | (SEQ ID NO: 124) |
| M34960 | TATA Box binding protein | 774 | GGTGGGGAGCTGTGATGTGAAGTTTCCTATAAGGTTA |
| | | | GAAGGCCTTGTGC |
| | | | (SEQ ID NO: 125) |
| NM_006258 | PRKG1 | 465 | CGGTGGAGTATGGCAAGGACAGTTGCATCATCAAAG |
| | | | AAGGAGACGTGGGG |
| | | | (SEQ ID NO: 126) |
| NM_002739 | PRKCG Protein kinase C, | 901 | CTGACGAAACAGAAGACCCGAACGGTGAAAGCCACG |
| | gamma | | CTAAACCCTGTGTG |
| | | | (SEQ ID NO: 127) |
| NM_002557 | Oviductal glycoprotein | 846 | GGACGTACCTTTCGCCTCCTCAAAGCCTCTAAGAATG |
| | | | GGTTGCAGGCCAG |
| | | | (SEQ ID NO: 128) |
| NM_173198 | (MINOR) Mitogen induced | 1055 | CCAATGGCCTCTTTCCTCCCAAATAAACCACTGGCTT |
| | nuclear orphan receptor | | TCTCTTTGTCCCC |
| | | | (SEQ ID NO: 129) |
| NM_173198 | (MINOR) Mitogen induced | 2957 | TGTTCTGCAATGGACTTGTCCTGCATCGACTTCAGTG |
| | nuclear orphan receptor | | CCTTCGTGGATTT |
| | | | (SEQ ID NO: 130) |
| NM_173198 | (MINOR) Mitogen induced | 2647 | CCACCTTCTCCTCCAATCTGCATGATGAATGCCCTTG |
| | nuclear orphan receptor | | TCCGAGCTTTAAC |
| | | | (SEQ ID NO: 131) |
| NM_173198 | (MINOR) Mitogen induced | 4095 | CCCTGTCGATCCCTTCTGAGGTATGGCCCATCCAAGA |
| | nuclear orphan receptor | | CTTTTAGGCCATT |
| | | | (SEQ ID NO: 132) |
| NM_012256 | Zinc-fmger protein C2H2- | 518 | GGTCACTGGAGAATGATGGCGTCTGTTTCACCGAGC |
| | 150 | | AGGAATGGGAGAAT |
| | | | (SEQ ID NO: 133) |
| NM_000867 | 5-Hydroxytryptamine 2B | 1809 | CGAAATGGGATTAACCCTGCCATGTACCAGAGTCCA |
| | receptor | | ATGAGGCTCCGAAG |
| | | | (SEQ ID NO: 134) |
| NM_001497 | Uncharacterized | 1868 | TCCAGGGCAACTCTAGCATCAGAGCAAAAGCCTTGG |
| | | | GTTTCTCGCATTCA |
| | | | (SEQ ID NO: 135) |
| NM_001752 | Catalase | 1148 | TTTTGCCTATCCTGACACTCACCGCCATCGCCTGGGA |
| | | | CCCAATTATCTTC |
| | | | (SEQ ID NO: 136) |
| NM_001770 | CD19 | 128 | GGAAGAGGGAGATAACGCTGTGCTGCAGTGCCTCAA |
| | | | GGGGACCTCAGATG |
| | | | (SEQ ID NO: 137) |
| NM_152866 | CD20 | 64 | AACAAACTGCACCCACTGAACTCCGCAGCTAGCATC |
| | | | CAAATCAGCCCTTG |
| | | | (SEQ ID NO: 138) |
| NM_000732 | CD3-delta | 410 | GCCGACACACAAGCTCTGTTGAGGAATGACCAGGTC |
| | | | TATCAGCCCCTCCG |
| | | | (SEQ ID NO: 139) |
| NM_001251 | CD68 | 667 | TTCCCCTATGGACACCTCAGCTTTGGATTCATGCAGG |
| | | | ACCTCCAGCAGAA |
| | | | (SEQ ID NO: 140) |
| unusable | poly dT | polyA | AAAAAAAAAAAAAAAAAAAAAAAAAAAAAAAAAA |
| | | tail | AAAAAAAAAAAAAAAA |
| | | | (SEQ ID NO: 141) |
| NM_000636 | Manganese superoxide | 659 | CCACTGCAAGGAACAACAGGCCTTATTCCACTGCTG |
| | dismutase | | GGGATTGATGTGTG |
| | | | (SEQ ID NO: 142) |
| AY963585 | Cytochrome oxidase | 524 | CCCTGCCATAACCCAATACCAAACGCCCCTCTTCGTC |
| | | | TGATCCGTCCTAA |
| | | | (SEQ ID NO: 143) |
| M34960 | TATA Box binding protein | 537 | CCTAAAGACCATTGCACTTCGTCGCCGAAACGCCGA |
| | | | ATATAATCCCAAGC |
| | | | (SEQ ID NO: 144) |
| M34960 | TATA Box binding protein | 461 | CAGCTTCGGAGAGTTCTGGGATTGTACCGCAGCTGCA |
| | | | AAATATTGTATCC |
| | | | (SEQ ID NO: 145) |
| M34960 | TATA Box binding protein | 774 | GGTGGGGAGCTGTGATGTGAAGTTTCCTATAAGGTTA |
| | | | GAAGGCCTTGTGC |
| | | | (SEQ ID NO: 146) |

Statistical Analysis:

Statistical analyses of the association of gene expression, as measured by Array Plate gNPA™ (qNPA) technology, with survival were performed on the 116 cases treated with CHOP-R and the 93 cases treated with CHOP or CHOP-like regimens alone. The logarithms of gene-expression values were standardized to have standard deviation equal to 1.

Initial evaluation of HTG results related to patient survival (univariate analysis, comparison between CHOP and R-CHOP treated case results):

Hazard ratios, 95% confidence intervals, and p-values for the univariate associations between standardized log gene expression levels and patient OS were obtained using Cox proportional hazard regression (Cox et al., Journal of the Royal Statistical Society B. 1972; B34:187-220). To account for the relatively large number of test statistics, the overall statistical significance of the set of hypothesis tests against a global null hypothesis of no association was calculated, by permutation resampling, based on the “tail strength” (TS) statistic (Taylor et al., Biostatistics. 2006; 7:167-181). A test of the overall statistical interaction of gene expression and treatment type was considered in a similar fashion.

Multivariate Analysis:

In an exploratory analysis of parsimonious multivariate models, a subset selection, which determines the “best” model based on the global score chi-squared statistic was utilized (Furnival et al., Technometrics. 1974; 16:499-511). Candidate genes used in the model building process were those achieving nominal p-values <0.05. The top 3 models for each of one, two, three, and four variable models were derived. For presentation purposes, for each factor included the overall best identified model, patients were categorized by high versus low gene expression (above or below the median value). Patients were then grouped according to the number of adverse risk factors, and survival was examined.

Adjustment of 2-Gene Model for Clinical IPI Score:

Finally, the ability of AP gene-risk model to retain significance of the biologic aspects of the malignant cells after adjusting for the clinically-based IPI index was assessed (Shipp et al., N Engl J. Med. 1993; 329:987-994).

Variable Cut Point Analysis on 2 Key Genes:

Separately, cut-point analysis was performed on the factors identified in multivariate modeling, in order to optimize identification of expression levels of highest risk. Permutation resampling was used to adjust significance levels of the proportional hazards score tests among all evaluated cut-points (LeBlanc et al., Assay Drug Dev Technol. 2002; 1:61-71). In addition, to control statistical variability of the cut-point analysis, a minimum possible group size of 10% of total patients was set for the analysis

Results

Performance of Assay in FFPET Blocks

Of 209 cases attempted, there was only 1 that did not result in adequately detectable signal. In situ hybridization using a polyDT probe (Ventana Medical Systems, Tucson, Ariz.) demonstrated that the mRNA was degraded (data not shown). This failure was therefore attributed to sample inadequacy rather than a technical failure of the assay itself. TBP was moderately and consistently expressed in all samples as in our previous work, and again used as the control gene for normalization of the data (Roberts et al., Laboratory Investigation. 2007; 87:979-997).

Overall Rationale and Sequence of Statistical Analyses

Initial evaluation of HTG results included univariate analysis of individual gene levels with respect to patient survival in both treatment groups, using the logarithm of the gene expression measurements. To further explore potentially important genes, hazard ratios of death were calculated. An assessment of whether the hazard ratios trended in the direction predicted by the previously reported literature was made. For each of the treatment groups, the overall significance of the panel of genes was assessed using the tail strength statistic and permutation resampling. Any gene which was significantly associated (p<0.05) with overall survival in univariate modeling was assessed for potential inclusion in a multivariate risk model using Cox regression analysis (Cox et al., 1972). To determine the best model, a subset selection, which determines the “best” model based on the global score chi-squared statistic, (reference Furnival) was determined. This model was adjusted for clinical IPI score. A variable cut point analysis was performed on the 2 key genes in order to see if there were more relevant cut-points, rather than the pre-selected 50th percentile, which might have biological implications. Permutation sampling was used to adjust for multiple comparisons in the cut-point optimization.

CHOP Results

For chemotherapy-alone (mainly CHOP) treated cases, gene expression levels were significantly correlated with overall survival at p<0.05 for 15/36 prognostic genes including the Major Histocompatibility Class II genes HLA-DR and HLA-DP; germinal center associated genes BCL6, GCET1 (SERPINA9), stromal associated genes (ACTN1, COL3A1, CTGF, FN1), proliferation genes MYC, CCND2, PRKCB1, as well as PDCD4, TLE, B4GALT1, and BCL-2. These genes represented all 4 prognostic signatures from Rosenwald et al., 4 of 13 genes reported by Shipp et al., and 3 of 6 genes from Lossos et al. An additional gene, CCL3, was borderline significant at 0.062.

R-CHOP Results

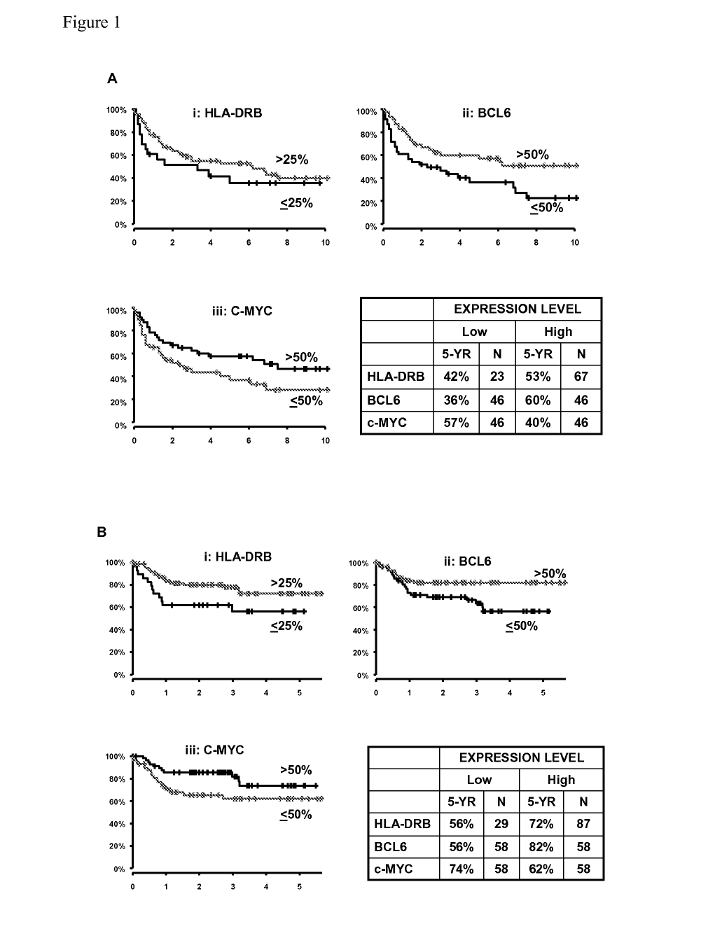

For the R-CHOP treated patients, 11 of the 36 genes analyzed were significantly associated with survival at the p<0.05 cut-off level. These genes were GCET1 (SERPINA9), HLA-DQA1, HLA-DRB, ACTN1, COL3A1, PLAU, MYC, BCL6, LMO2, PDCD4, and SOD2. An additional gene, FN1, was marginally significant at a p-value of 0.078. Results of univariate analyses compared for the 2 treatment eras are shown side by side in TABLE 3. To emphasize the genes with recurrent significance, the p-values at 0.05 or less are highlighted in bold font with grey shading while p-values between 0.1 and 0.05 are highlighted in grey shading. Average 2-year overall survival for each gene cut at above and below the median expression level are also summarized in TABLE 3. It should be noted that survival rates at 2 years were chosen as simple descriptive summary statistics. Similar results were seen with 3 year and 4 year rates, although estimates were more unstable due to more censored cases. The p-values presented in the tables are based on Cox score tests using the continuous logarithm of gene expression and therefore do not depend on the choice of summary survival estimates presented. Comparative overall survival curves in the different treatment eras for HLA-DRB (an MHC Class II gene), BCL6, and MYC are demonstrated in FIG. 1. These examples demonstrate the ability of the ArrayPlate assay to generate meaningful quantitative data that can be related to patient outcome. The results also demonstrate that for these well-known prognostic genes, there is continued evidence of prognostic relevance in R-CHOP treated patients.

TABLE 3
Results of Univariate Analyses of Gene Expression with Overall Survival

| | CHOP | | | CHOP + R | | |
|---|---|---|---|---|---|---|
| | | | 2-yr OS (split | | | 2-yr OS (split |
| Gene | HR | p-value* | at < vs. ≧ median) | HR | p-value | at < vs. ≧ median) |
| BCL6 | 0.65 | 0.008 | 69%, 52% | 0.62 | 0.007 | 82%, 69% |
| GCET1 | 0.75 | 0.01 | 69%, 53% | 0.62 | 0.013 | 83%, 68% |
| (SERPINA9) | | | | | | |
| GCET2 | 0.93 | 0.608 | 59%, 66% | 0.93 | 0.608 | 77%, 74% |
| HLA-DPA1 | 0.71 | 0.036 | 63%, 58% | 0.77 | 0.115 | 84%, 68% |
| HLA-DQA1 | 1.14 | 0.35 | 63%, 62% | 0.65 | 0.020 | 83%, 68% |
| HLA-DRA | 0.72 | 0.02 | 63%, 58% | 0.91 | 0.580 | 84%, 68% |
| HLA-DRB | 0.98 | 0.921 | 58%, 66% | 0.71 | 0.030 | 89%, 62% |
| ACTN1 | 0.66 | 0.03 | 73%, 49% | 0.62 | 0.011 | 85%, 66% |
| COL3A1 | 0.78 | 0.016 | 71%, 50% | 0.67 | 0.029 | 81%, 69% |
| CTGF | 0.79 | 0.026 | 75%, 45% | 0.80 | 0.211 | 84%, 67% |
| FN1 | 0.77 | 0.01 | 67%, 54% | 0.73 | 0.078 | 78%, 73% |
| FAM38A | 1.16 | 0.462 | 61%, 60% | 0.85 | 0.426 | 80%, 71% |
| PLAU | 0.73 | 0.122 | 68%, 54% | 0.56 | 0.001 | 84%, 67% |
| MYC | 1.40 | 0.047 | 54%, 67% | 1.64 | 0.007 | 65%, 86% |
| C20ORF155 | 1.32 | 0.369 | 52%, 70% | 1.03 | 0.851 | 72%, 79% |
| NPM3 | 1.27 | 0.25 | 58%, 64% | 1.22 | 0.303 | 74%, 76% |
| BMP6 | 1.26 | 0.216 | 64%, 60% | 0.86 | 0.402 | 77%, 74% |
| LMO2 | 1.03 | 0.832 | 57%, 64% | 0.62 | 0.011 | 82%, 69% |
| BCL2 | 1.44 | 0.018 | 57%, 66% | 1.11 | 0.569 | 71%, 80% |
| CCL3 | 1.40 | 0.062 | 48%, 73% | 0.82 | 0.296 | 84%, 67% |
| CCND2 | 1.45 | 0.002 | 53%, 69% | 1.23 | 0.271 | 76%, 75% |
| DRP2 | 1.02 | 0.878 | 66%, 60% | 0.94 | 0.719 | 75%, 76% |
| PRKCB1 | 1.47 | 0.028 | 51%, 71% | 0.99 | 0.951 | 79%, 73% |
| PDCD4 | 1.89 | 0.001 | 50%, 73% | 1.53 | 0.023 | 67%, 84% |
| MAP1B | 1.05 | 0.772 | 63%, 64% | 0.94 | 0.717 | 78%, 72% |
| TLE1 | 1.60 | 0.001 | 42%, 80% | 1.16 | 0.428 | 70%, 81% |
| SLC25A13 | 1.15 | 0.676 | 58%, 63% | 0.89 | 0.540 | 78%, 73% |
| PDE4B | 1.19 | 0.423 | 56%, 64% | 1.17 | 0.402 | 75%, 76% |
| B4GALT1 | 1.87 | 0.001 | 49%, 71% | 0.82 | 0.258 | 80%, 72% |
| PRKCG | 1.05 | 0.701 | 49%, 72% | 1.02 | 0.924 | 77%, 71% |
| OVGP1 | 1.25 | 0.279 | 52%, 73% | 1.02 | 0.924 | 77%, 71% |
| NR4A3 | 1.30 | 0.151 | 49%, 71% | 0.80 | 0.227 | 72%, 80% |
| ZNF212 | 0.99 | 0.965 | 58%, 64% | 1.04 | 0.810 | 73%, 78% |
| HTR2B | 1.04 | 0.834 | 48%, 68% | 0.76 | 0.210 | 81%, 64% |
| CAT | 1.24 | 0.50 | 54%, 67% | 0.99 | 0.962 | 80%, 71% |
| SOD2 | 1.10 | 0.573 | 60%, 61% | 0.64 | 0.014 | 87%, 64% |

*P-values at 0.05 or less are highlighted in bold font.
2-yr OS above/below median presented for illustrative purposes

For most genes in both treatment groups, the estimated hazard ratios of death trended in the direction predicted by the original studies (TABLE 4). Hazard ratios (HR) correspond to a change in one standard deviation in log expression levels and a HR above one indicate an association between high expression (above the median) with poorer outcome, while hazard ratios below one indicate an association between high expression with better outcome. Therefore, an estimated HR that is very small in magnitude (e.g., close to zero) corresponds to a gene with strong association between higher expression and longer survival.

TABLE 4
Hazard Ratios of Overall Survival in R-CHOP Treated Patients and
Agreement with Original Study in Regards to Predictive Capacity.

| | | Agree with trend in |
|---|---|---|
| Gene* | HR (95% CI) | original study |
| BCL6 | 0.62 (0.44-0.87) | yes |
| GCET1 (SERPINA9) | 0.62 (0.41-0.92) | yes |
| GCET2 | 0.93 (0.66-1.31) | yes |
| HLA-DPA1 | 0.77 (0.56-1.05) | yes |
| HLA-DQA1 | 0.65 (0.45-0.93) | yes |
| HLA-DRA | 0.91 (0.67-1.24) | yes |
| HLA-DRB | 0.71 (0.53-0.95) | yes |
| ACTN1 | 0.62 (0.43-0.89) | yes |
| COL3A1 | 0.67 (0.47-0.97) | yes |
| CTGF | 0.80 (0.56-1.14) | yes |
| FN1 | 0.73 (0.51-1.04) | yes |
| FAM38A | 0.85 (0.58-1.26) | yes |
| PLAU | 0.56 (0.40-0.79) | yes |
| MYC | 1.64 (1.16-2.31) | yes |
| C20ORF155 | 1.03 (0.73-1.47) | no |
| NPM3 | 1.22 (0.84-1.76) | yes |
| BMP6 | 0.86 (0.59-1.23) | no |
| LMO2 | 0.62 (0.43-0.90) | yes |
| BCL2 | 1.11 (0.77-1.60) | yes |
| CCL3 | 0.82 (0.58-1.18) | no |
| CCND2 | 1.23 (0.85-1.77) | yes |
| DRP2 | 0.94 (0.65-1.35) | yes |
| PRKCB1 | 0.99 (0.69-1.42) | no |
| PDCD4 | 1.53 (1.07-2.21) | yes |
| MAP1B | 0.94 (0.66-1.33) | yes |
| TLE1 | 1.16 (0.81-1.65) | yes |
| SLC25A13 | 0.90 (0.63-1.28) | yes |
| PDE4B | 1.17 (0.81-1.68) | yes |
| B4GALT1 | 0.82 (0.57-1.16) | no |
| PRKCG | 1.02 (0.69-1.51) | yes |
| OVGP1 | 1.03 (0.73-1.47) | yes |
| NR4A3 | 0.80 (0.55-1.15) | yes |
| ZNF212 | 1.04 (0.74-1.48) | yes |
| HTR2B | 0.76 (0.49-1.18) | yes |
| CAT | 0.99 (0.70-1.41) | no |
| SOD2 | 0.64 (0.45-0.92) | yes |

HR = Hazard Ratio; CI = Confidence Interval
Interpretation: Hazard ratios = 1 indicate no effect on risk. Hazard ratios between 0 and 1 indicate good risk. Hazard ratios greater than 1 indicate poor risk.
*Standardized to Normal(0, 1) distribution.

Comparison of CHOP and R-CHOP Data

To address the testing of the multiple genes in the panel, an overall test of the 36 p-values was performed using the tail strength (TS) statistic and permutation resampling. There is evidence of association between the overall 36-gene panel and outcome in both CHOP treated patients (TS: p=0.007) and R-CHOP patients (TS: p=0.013) (Taylor et al., Biostatistics. 2006; 7:167-181). An overall test of differences by treatment group in the association between each gene and survival (statistical interaction) was also considered. While power for interaction testing is limited, there was no evidence of a differential effect of the overall 36-gene expression panel between the two treatment types (TS: p=0.250).

As an overall assessment of important prognostic features the IPI distribution was assessed among patients in the two treatment types. In the CHOP alone patients, 41% had IPI of 0-1, 48% had IPI of 2-3, and 11% had IPI of 4-5. In the CHOP-R treated patients, 40% had IPI of 0-1, 56% had IPI of 2-3, and 4% had IPI of 4-5. There was no evidence of a difference between the 2 treatment groups (p=0.18). TABLE 5 details the distribution of the individual factors of the IPI score between patients in the 2 treatment eras.

TABLE 5
Distribution of factors in the International Prognostic
Index (IPI) between patients in the 2 treatment eras

| IPI Factor | CHOP | R_CHOP |
|---|---|---|
| Age >60 years | 47% | 49% |
| LDH > Upper limit of normal | 54% | 60% |
| Stage >II | 48% | 60% |
| >1 Extra Nodal Site | 17% | 17% |
| Performance Status .1 | 16% | 29% |

TABLE 6
List of control genes

| Abbreviation | Full Name |
|---|---|
| PRKG1 | protein kinase, cGMP-dependent, type I |
| CD19 | CD 19 molecule |
| MS4A1 | membrane-spanning 4-domains, subfamily A, member 1 |
| CD3 delta | CD3d molecule, delta (CD3-TCR complex) |
| CD68 | CD68 molecule |
| CYTOX+ | Cytochrome oxidase |

Prognostic Model

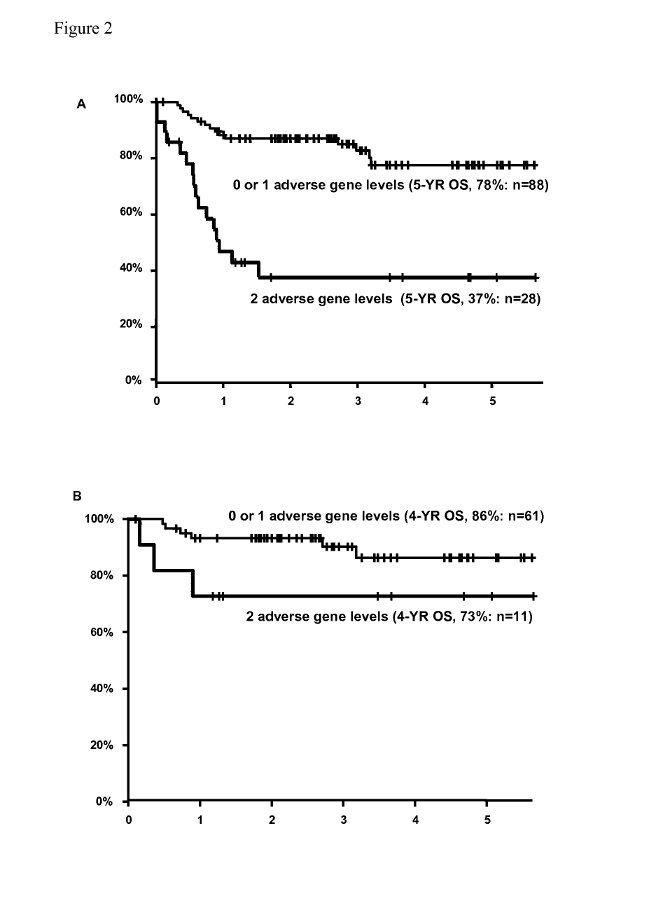

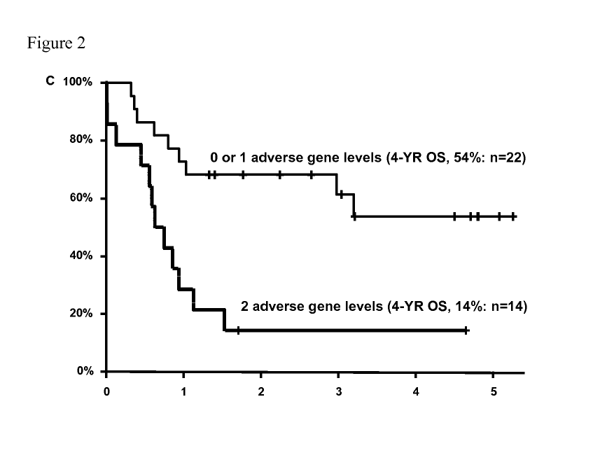

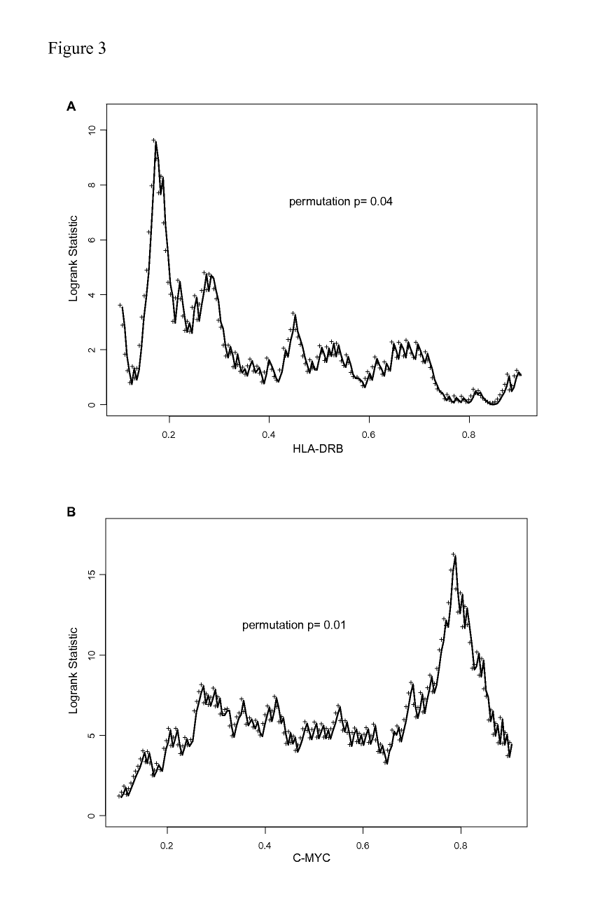

As an exploratory analysis of multivariable prognostic models, best one, two, three, and four variable models, as determined by best subsets analysis were calculated (data not shown). The best 2-variable model was the combination of MYC and HLA-DRB, with a model chi-square of 16.6. However, it was noted that other 2-variable models, including MYC with HLA-DQA1 or PLAU had modestly smaller model chi-square statistics. Given the relatively small number of events in this study, conclusive statements about the overall best model are not possible. There was no evidence that 3 variable models yielded any statistical improvement in model fit. Patients were defined as having high or low levels of MYC and HLA-DRB. Twenty-eight patients (24%) had both adverse gene levels. These patients had much worse survival than patients with 0 or 1 adverse gene level (2-year overall survival 38% vs. 87%) as shown in FIG. 2A. Differences are presented for both the high and low IPI subgroups (FIGS. 2B-C). The survival disadvantage for patients with both adverse gene levels appears particularly pronounced in patients with high IPI (2-year estimate, 14% vs. 68%), although there was no evidence of an interaction between number of adverse gene levels and IPI group (p=0.88). Both CHOP and R-CHOP data were combined to further explore the nature of the association of expression of these 2 genes with survival using cutpoint analysis. For HLA-DRB, the highest chi-square value indicating the most significant cut point was at the lower 20th percentile of gene expression (p=0.01 based on permutation resampling to account for the multiple testing). For MYC, the most significant cut point (p=0.01) was at the upper 80th percentile of expression (FIG. 3). Given the adaptive nature of cutpoint selection, any multivariate model based on cutpoint levels identified in this analysis would be best validated independently. It should be emphasized that while the 80th percentile was the optimal cutpoint for MYC (corresponding to a chi-square value of >15 and a nominal p<0.0001), there were a wide range of cut-point values that were also nominally significant (p<0.025). This indicates other cut-points may lead to interesting prognostic models.

Additional CMYC, HLA-DR Analyses

Additional analyses were conducted to consider modified cut points on HLA-DRB and CMYC. Cut points used in the further studies were lower 35th percentile of gene expression for HLA-DRB and upper 30th percentile of gene expression for MYC. These analyses (data not shown) indicate that the combination of either adverse HLA-DRB or adverse Myc gene expression with an adverse IPI score of 4 to 5, results in the prognosis of a survival outcome of 20%, whereas IPI scores alone of 4 to 5 predict 40% survival, demonstrating the improved prognostic value of the current method.

The model presented for MYC and HLA-DR can be used as a template to derive prognostic models based on other gene combinations. The same algorithm applies for any other multivariate gene model among the 16 selected genes. A cut-point is specified based on either the median value or the value optimizing the two-sample logrank test statistic. This cut-point rule defines 2 groups (ie good versus poor performers) for any gene and an overall prognostic groups is derived by the counting the number of poor prognostic attributes. In the clinical setting, prediction of prognosis for a newly diagnosed patient would depend on the number of poor prognostic attributes. Modeling strategies including (but not limited to) proportional hazards regression, lasso regression or extreme regression may be used as alternatives for prognostic rules.

Below is a table of the top 12 models of the 55 possible combinations from 11 univariate significant genes, indicating that all 11 genes are included in at least one pairwise model (ie: each of the eleven genes is present in at least one of the top 12 prognostic marker pairs. Each pairwise model shows statistical significance (unadjusted prognostic p-value).

TABLE 7

| Rank Order | | | | |
|---|---|---|---|---|
| (by Chisquare | | | Chisquare | Overall Model |
| Statistic) | Gene 1 | Gene 2 | Statistic | P-value |
| 1 | HLA-DRB | c-MYC | 16.65 | .0002 |
| 2 | HLA-DQA1 | c-MYC | 14.40 | .0007 |
| 3 | PLAU | c-MYC | 13.25 | .0013 |
| 4 | COL3A1 | c-MYC | 13.04 | .0015 |
| 5 | c-MYC | SOD2 | 12.53 | .0019 |
| 6 | ACTN1 | c-MYC | 12.39 | .0020 |
| 7 | c-MYC | BCL6 | 12.38 | .0020 |
| 8 | SERPINA9 | c-MYC | 11.96 | .0025 |
| 9 | PLAU | BCL6 | 11.69 | .0029 |
| 10 | PLAU | PDCD4 | 11.26 | .0036 |
| 11 | SERPINA9 | PLAU | 11.15 | .0038 |
| 12 | PLAU | LMO2 | 10.81 | .0045 |

We then compared prognostic results from the current study to patients receiving CHOP+R as reported by Losso et. al (2008) and Lenz et. al (2008). Formal comparisons of the performance of the prognostic models are not possible given the raw data are not available. However, based on the estimated survival curves, the Rimsza et. al results perform well in terms of differences between good and poor risk groups.
1. Current study Good Risk: 5 year OS, 78% (n=88); Poor Risk: 5 year OS, 37% (n=28)
2. Lenz et al.: Quartile 1: 3 year OS, 89% (n=58); Quartile 2: 3 year OS, 82% (n=58): Quartile 3: 3 year OS, 74% (n=59); Quartile 4: 3 year OS, 48% (n=58)
3. Lossos et al.: Good Risk: 2 year OS, 85% (n=67); Poor Risk: 2 year OS, 61% (n=65)

Note, the results presented vary in important ways, including the number of cases, the model building strategies, the number of cases in the poor and good risk groups and the timepoints for estimated overall survival. To make the results more comparable to our analysis we average the survival results for the Quartiles 1-3 (so that approximately ¾ of the cases are in the good risk group). Note that this is a crude average since we do not have the raw data. Next, for each of three papers, OS is reported at different times. We report the crude hazard ratio as a measure of the difference in prognosis between the groups. We approximate this number by the log(OS good prognosis)/log(OS poor prognosis).
1. Current study HR=4.0
2. Lenz et al. HR=3.6
3. Lossos et al. HR=3.0

Larger hazard ratios should be associated with greater strength of prognostic association. However, it should be noted that the current model used fewer variables.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above and in the following list are incorporated herein by reference.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

### REFERENCE LIST

(1) Rosenwald A, Wright G, Chan W C et al. The use of molecular profiling to predict survival after chemotherapy for diffuse large-B-cell lymphoma. N Engl J. Med. 2002; 346:1937-1947.
(2) Tome M E, Johnson DBF, Rimsza L M et al. A redox signature score identifies diffuse large B-cell lymphoma patients with a poor prognosis. Blood. 2005; 106:3594-3601.
(3) Shipp M A, Ross K N, Tamayo P et al. Diffuse large B-cell lymphoma outcome prediction by gene-expression profiling and supervised machine learning. Nat Med. 2002; 8:68-74.
(4) Lossos I S, Czerwinski D K, Alizadeh A A et al. Prediction of survival in diffuse large-B-cell lymphoma based on the expression of six genes. N Engl J. Med. 2004; 350:1828-1837.
(5) Roberts R A, Sabalos C M, LeBlanc M L et al. Quantitative nuclease protection assay in paraffin-embedded tissue replicates prognostic microarray gene expression in diffuse large-B-cell lymphoma. Laboratory Investigation. 2007; 87:979-997.
(6) Coiffier B. State-of-the-art therapeutics: Diffuse large B-cell lymphoma. Journal of Clinical Oncology. 2005; 23:6387-6393.
(7) Sehn L, Donaldson J, Chhanabhai M, Fitzgerald C, Gill K, Klasa R et al. Introduction of combined CHOP plus rituximab therapy dramatically improved outcome of diffuse large B-cell lymphoma in British Columbia. Journal of Clinical Oncology 23[22], 5027-5033. Aug. 1, 2005.
(8) Habermanb T M, Weller E A, Morrison V A, Gascoyne R D, Cassileth P A, Cohn J B et al. Rituximab-CHOP versus CHOP alone or with maintenance rituximab in older patients with diffuse large B-cell lymphoma. Journal of Clinical Oncology 24[19], 3121-3127. Jul. 1, 2006.
(9) Winter J N, Weller E A, Horning S J et al. Prognostic significance of Bcl-6 protein expression in DLBCL treated with CHOP or R-CHOP: a prospective correlative study. Blood. 2006; 107:4207-4213.
(10) Mounier N, Briere J, Gisselbrecht C et al. Rituximab plus CHOP (R-CHOP) in the treatment of elderly patients with diffuse large B-cell lymphoma (DLBCL) overcomes Bcl2-associated chemotherapy resistance. Blood. 2002; 100:161A.
(11) Lenz G, Wright G, Dave S et al. Gene Expression Signatures Predict Overall Survial in Diffuse Large B Cell Lymphoma Treated with Rituximab and Chop-Like Chemotherapy [abstract]. Blood. 2007; 110:109a.
(12) Lossos I S, Czerwinski D K, Wechser M A, Levy R. Optimization of quantitative real-time RT-PCR parameters for the study of lymphoid malignancies. Leukemia. 2003; 17:789-795.
(13) Cox D. Regression models and life tables. Journal of the Royal Statistical Society B. 1972; B34:187-220.
(14) Taylor J, Tibshirani R. A tail strength measure for assessing the overall univariate significance in a dataset. Biostatistics. 2006; 7:167-181.
(15) Furnival G M, Wilson R W. Regressions by Leaps and Bounds. Technometrics. 1974; 16:499-511.
(16) The International Non-Hodgkin's Lymphoma Prognostic Factors Project. A predictive model for aggressive non-Hodgkin's lymphoma. The International Non-Hodgkin's Lymphoma Prognostic Factors Project. N Engl J. Med. 1993; 329:987-994.
(17) LeBlanc M, Crowley J. Step-Function Covariate Effects in the Proportional-Hazards Model. Canadian Journal of Statistics-Revue Canadienne de Statistique. 1995; 23:109-129.
(18) Martel R R, Botros I W, Rounseville M P et al. Multiplexed screening assay for mRNA combining nuclease protection with luminescent array detection. Assay Drug Dev Technol. 2002; 1:61-71.
(19) Berk A J, Sharp P A. Sizing and Mapping of Early Adenovirus Messenger-Rnas by Gel-Electrophoresis of S1 Endonuclease-Digested Hybrids. Cell. 1977; 12:721-732.
(20) Sawada H, Taniguchi K, Takami K. Improved toxicogenomic screening for drug-induced phospholipidosis using a multiplexed quantitative gene expression ArrayPlate assay. Toxicol In Vitro. 2006; 20:1506-1513.
(21) Natkunam Y, Farinha P, Hsi E D et al. LMO2 Protein Expression Predicts Survival in Patients with Diffuse Large B-Cell Lymphoma in the Pre- and Post-Rituximab Treatment Eras [abstract]. Blood. 2007; 110:24a.
(22) Malumbres R, Johnson N A, Sehn L H et al. Paraffin-Based 6-Gene Model Predicts Outcome of Diffuse Large B-Cell Lymphoma Patients Treated with R-CHOP [abstract]. Blood. 2007; 110:23a.
(23) Rimsza L M, Roberts R A, Miller T P et al. Loss of MHC class II gene and protein expression in diffuse large B-cell lymphoma is related to decreased tumor immunosurveillance and poor patient survival regardless of other prognostic factors: a follow-up study from the Leukemia and Lymphoma Molecular Profiling Project. Blood. 2004; 103:4251-4258.
(24) Rimsza L M, Farinha P, Fuchs D A et al. HLA-DR protein status predicts survival in patients with diffuse large B-cell lymphoma treated on the MACOP-B chemotherapy regimen. Leuk Lymphoma. 2007; 48:542-546.

We claim:

1. A method of prognosticating an outcome of treatment for diffuse large B cell lymphoma (DLBCL) in a patient comprising:
obtaining a test sample from a patient with DLBCL;
detecting a level of expression products of between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein a level of expression product of no more than sixteen genes in total is detected; and
comparing an expression product level of the genes in the test sample with an expression product level of the genes in a control;
wherein the expression product levels of the genes in the test sample compared to the expression product levels of the gene in a control is prognostic for an outcome of treatment for the patient with DLBCL if treated with combination chemotherapy.

2. The method of claim 1, wherein the combination chemotherapy comprises a combination of cyclophsophamide, oncovorin, prednisone, and one or more chemotherapeutics selected from the group consisting of hydroxydaunorubicin, epirubicin, and motixantrone.

3. The method of claim 1, wherein the combination chemotherapy further comprises monoclonal antibody therapy.

4. The method of claim 1, wherein the monoclonal antibody therapy comprises rituximabanti-CD20 monocloncal antibody therapy.

5.-14. (canceled)

15. The method of claim 1, wherein a level of expression product of no more than twelve genes in total is detected.

16. A method of prognosticating an outcome of treatment for diffuse large B cell lymphoma (DLBCL) in a patient comprising:
obtaining a test sample from a patient with DLBCL;
detecting a level of expression products of at least one gene selected from the group consisting of GCET1, HLA-DQA1, HLA-DRB, HLA-DRA, ACTN1, COL3A1, PLAU, MYC, BCL6, LMO2, PDCD4, and SOD2; and
comparing an expression product level of the genes in the test sample with an expression product level of the genes in a control;
wherein the expression product levels of the genes in the test sample compared to the expression product levels of the gene in a control is prognostic for an outcome of treatment for the patient with DLBCL if treated with monoclonal antibody therapy together with combination chemotherapy.

17. The method of claim 16, wherein the combination chemotherapy comprises a combination of cyclophsophamide, oncovorin, prednisone, and one or more chemotherapeutics selected from the group consisting of hydroxydaunorubicin, epirubicin, and motixantrone.

18. The method of claim 16, wherein the monoclonal antibody therapy comprises anti-CD20 monoclonal antibody therapy.

19-27. (canceled)

28. The method of claim 16, wherein a level of expression product of no more than twelve genes in total is detected.

29. A method of prognosticating an outcome of treatment for diffuse large B cell lymphoma (DLBCL) in a patient comprising:
obtaining a test sample from a patient with DLBCL;
detecting a level of expression products of between one and twelve genes selected from the group consisting of GCET1, HLA-DQA1, HLA-DRB, HLA-DRA, ACTN1, COL3A1, PLAU, MYC, BCL6, LMO2, PDCD4, and SOD2;
determining an international prognostic index (IPI) score for the patient; and
comparing an expression product level of the genes in the test sample with an expression product level of the genes in a control;
wherein the expression product levels of the genes in the test sample compared to the expression product levels of the gene in a control, in combination with an IPI score for the patient, is prognostic for an outcome of treatment for the patient with DLBCL if treated with combination chemotherapy.

30. The method of claim 29, wherein the combination chemotherapy comprises a combination of cyclophsophamide, oncovorin, prednisone, and one or more chemotherapeutics selected from the group consisting of hydroxydaunorubicin, epirubicin, and motixantrone.

31. The method of claim 29, wherein the combination chemotherapy further comprises monoclonal antibody therapy.

32. The method of claim 29, wherein the monoclonal antibody therapy comprises rituximabanti-CD20 monocloncal antibody therapy.

33-40. (canceled)

41. The method of claim 29, wherein a level of expression product of no more than twelve genes in total is detected.

42. The method of claim 29, wherein the expression product levels of the genes in the test sample compared to the expression product levels of the gene in a control, in combination with an IPI score of 4 to 5 for the patient, is prognostic for an outcome of treatment for the patient with DLBCL if treated with combination chemotherapy.

43. The method of claim 42, wherein the expression product level of one or both of MYC and HLA-DRB are detected.

44. A composition, comprising probes for expression products from between two and twelve genes selected from the group consisting of GCET1, HLA-DQA1, HLA-DRB, HLA-DRA, ACTN1, COL3A1, PLAU, MYC, BCL6, LMO2, PDCD4, and SOD2, wherein the probes are selected from the group consisting of oligonucleotide probes, antibody probes, oligonucleotide primer pairs, and aptamers, and wherein the probes are optionally detectably labeled.

45. The composition of claim 44, wherein the composition consists of probes for no more than sixteen different gene expression products.

46-51. (canceled)

52. A kit, comprising the composition of claim 44 and instructions for its use in prognosing a treatment outcome for DLBCL patient.
